(19) United States
(12) Patent Application Publication
(10) Pub. No.: US 2022/0236257 A1
(43) Pub. Date: Jul. 28, 2022

(54) ARTIFICIAL RECEPTORS, RECOMBINANT CELLS COMPRISING THEREOF, METHODS FOR THEIR PREPARATION, AND METHOD OF USING THEREOF

(71) Applicant: YEDA RESEARCH AND DEVELOPMENT CO. LTD., Rehovot (IL)

(72) Inventors: David MARGULIES, Rehovot (IL); Leila MOTIEI, Rehovot (IL); Naama LAHAV, Rehovot (IL)

(73) Assignee: YEDA RESEARCH AND DEVELOPMENT CO. LTD., Rehovot (IL)

(21) Appl. No.: 17/614,563

(22) PCT Filed: Jun. 5, 2019
(86) PCT No.: PCT/IL2019/050639

§ 371 Date: Nov. 29, 2021

(51) Int. Cl. G01N 33/532; C07K 14/705; G01N 33/50; C12N 5/00; C12N 1/00; C12Q 1/02

(52) U.S. Cl. CPC G01N 33/532; C07K 14/705; G01N 33/5008; C12N 5/0006; C07K 2319/33; C12Q 1/025; C07K 2319/03; C07K 2319/21; C12N 1/005

(57) ABSTRACT

The disclosure presented herein provides artificial receptors and a system comprising recombinant cells decorated with various labels and/or synthetic agents, wherein said labels and/or synthetic agents can be reversibly modified or removed from the cells. Also disclosed herein are methods for decorating and/or modifying the cells and methods for using thereof.

### FIELD OF THE DISCLOSURE

The disclosure presented herein provides a system comprising recombinant cells decorated with various labels and/or synthetic agents, wherein said labels and/or synthetic agents can be reversibly modified or removed from the cells. Also disclosed herein are methods for decorating and/or modifying the cells and methods for using thereof.

### BACKGROUND

Various cellular processes are mediated by the function of cell surface proteins (CSPs) that, by binding to various extracellular signals such as ions, small molecules, proteins, and cells, control the response of cells to their surroundings. These interactions, which allosterically change CPS' structures, can mediate physical processes such as adhesion or transport. In addition, the CPS-ligand interactions can trigger subsequent cell signal cascades that eventually alter the cell's behavior. Bacterial adhesins, for example, can mediate non-specific adhesion to solid supports, can selectively interact with host cells and trigger the expression of virulence genes that provide the bacteria with pathogenic properties. Another example of the way CSP responses can induce new cellular functions is the binding of quorum sensing (QS) receptors to small molecules autoinducers (AIs). In this case, QS receptor activation by AIs induces a collective gene expression behavior that ultimately results in the formation of biofilm or bioluminescence.

To enable the cells to dynamically adapt to changes in their environments, CSPs' functions are tightly regulated. Regulation is achieved due to the reversibility of the CSPs-ligand interactions, which makes them dependent on the concentrations of the external cues. In addition, the response of cells to their environment is controlled by feedback loops that dynamically alter CSPs' structure, local concentration, and/or composition. For instance, following host infection, adhesins undergo posttranslational modifications (PTMs) that can disrupt inter-bacterial adhesion. Similarly, activation of QS receptors by AIs differentially changes the receptors' expression levels. These examples highlight the complex interplay between CSPs and external molecular signals. The latter reversibly interact with CSPs, change their conformation, and trigger subsequent cell signal cascades that can further alter CSPs' structure, composition, and expression levels.

In recent years, considerable attention has been devoted to developing oligodeoxynucleotide (ODN)-small molecule conjugates that, similar to CSPs, can respond to external stimuli and undergo dynamic structural changes that enables them to reversibly interact with proteins and mediate their functions. Similarly, cell surfaces were modified by attaching synthetic agents to them.

It is clear that there remains a critical need for systems capable of decorating or coating cell membranes with proteins of interest. Ideally, such a system would allow easy proteins attachment to the cell membrane, and would allow controlling the structure, composition, binding interactions, and concentrations of said proteins on the cell membrane. Further, an ideal system would allow controlling and reversing such parameters by easily administering extremal chemical signals. Such a system would provide a great level of control over cellular processes in vitro or in the living organism.

### SUMMARY OF THE INVENTION

In some embodiments, this invention relates to a system comprising:
a. a recombinant cell ectopically expressing a polypeptide, wherein said polypeptide comprises a membranal anchoring domain and an extracellular binding domain,
b. a first compound comprising a first oligonucleotide (ODN-1) covalently bound to a binder, either directly or through a first linker, said binder comprising affinity to said extracellular binding domain, and
c. a second compound comprising a second oligonucleotide (ODN-2) covalently bound to a synthetic agent, either directly or through a second linker, wherein said second oligonucleotide is complementary to said first oligonucleotide

In some embodiments, this invention relates to a recombinant cell ectopically expressing a polypeptide, wherein said polypeptide comprises a membranal anchoring domain and an extracellular binding domain, said extracellular binding domain bound to
a. a first compound comprising a first oligonucleotide (ODN-1) covalently bound to a binder, either directly or through a first linker, said binder comprising affinity to said extracellular binding domain,
b. a second compound comprising a second oligonucleotide (ODN-2) covalently bound to a bioactive moiety, either directly or through a second linker, wherein said second oligonucleotide is complementary to said first oligonucleotide.

In some embodiments, this invention relates to an artificial receptor, capable of binding a His-tagged protein, comprising
a. a first compound comprising a first oligonucleotide (ODN-1) covalently bound to a His-tag binder, either directly or through a first linker, said binder comprises a moiety represented by the structure of formula E:

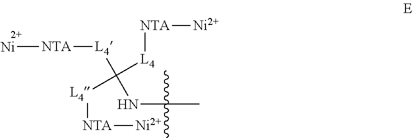

wherein
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof; and
b. a second compound comprising a second oligonucleotide (ODN-2) covalently bound to a synthetic agent, either directly or through a second linker, said second oligonucleotide is complementary to said first oligonucleotide.

In some embodiments, this invention relates to a method for decorating a cell with a synthetic agent, said method comprises:
a. ectopically expressing in said cell a polypeptide, wherein said polypeptide comprises a membranal anchoring domain and an extracellular binding domain,
b. incubating the cell of (a) with a first compound comprising a first oligonucleotide (ODN-1) covalently bound to a binder, either directly or through a first linker, said binder comprising affinity to said extracellular binding domain, and
c. incubating the cell of (b) with a second compound comprising a second oligonucleotide (ODN-2) covalently bound to a synthetic agent, either directly or through a second linker, wherein said second oligonucleotide is complementary to said first oligonucleotide;
   thereby decorating said cell with said synthetic agent.

In some embodiments, this invention relates to a method for binding a first cell to a second cell, said method comprises:
a. ectopically expressing in the first cell a polypeptide, wherein said polypeptide comprises a membranal anchoring domain and an extracellular binding domain,
b. incubating the cell of (a) with a first compound comprising a first oligonucleotide (ODN-1) covalently bound to a binder, either directly or through a first linker, said binder comprises affinity to said extracellular binding domain, and
c. incubating the cell of (b) with a second compound comprising a second oligonucleotide (ODN-2) covalently bound to an adhesion molecule, either directly or through a second linker, wherein said second oligonucleotide is complementary to said first oligonucleotide, and said adhesion molecule comprises affinity to a compound present on the surface of said second cell,
d. incubating said first cell with said second cell,
   thereby adhering said first cell to said second cell.

In some embodiments, this invention relates to a method for adhering a cell to an abiotic surface, said method comprises:
a. ectopically expressing in a cell a polypeptide, wherein said polypeptide comprises a membranal anchoring domain and an extracellular binding domain,
b. incubating the cell of (a) with a first compound comprising a first oligonucleotide (ODN-1) covalently bound to a binder, either directly or through a first linker, said binder comprises affinity to said extracellular binding domain,
c. incubating the cell of (b) with a second compound comprising a second oligonucleotide (ODN-2) covalently bound to an abiotic surface binder, either directly or through a second linker, wherein said second oligonucleotide is complementary to said first oligonucleotide, and said surface binder is capable of binding to said surface, and
d. applying said cell to said surface under conditions sufficient for the binding of said abiotic surface binder to said abiotic surface,
   thereby adhering said cell to said abiotic surface.

In some embodiments, this invention relates to a method for inducing luminescence in a cell, said method comprises:
a. ectopically expressing in a cell a first polypeptide, wherein said polypeptide comprises a membranal anchoring domain and an extracellular binding domain,
b. incubating the cell of (a) with a first compound, comprising a first oligonucleotide (ODN-1) covalently bound to a binder, either directly or through a first linker, said binder comprises affinity to said extracellular binding domain, and
c. incubating the cell of (b) with a second compound, comprising a second oligonucleotide (ODN-2) covalently bound to a luminescent molecule, either directly or through a second linker, wherein the second oligonucleotide is complementary to the first oligonucleotide,
   thereby inducing luminescence in said cell.

In some embodiments, this invention relates to a method for binding a cell to a protein of interest (POI), said method comprises:
a. ectopically expressing in a cell a polypeptide, wherein said polypeptide comprises a membranal anchoring domain and an extracellular binding domain,
b. incubating the cell of (a) with a first compound according to this invention, comprising a first oligonucleotide (ODN-1) covalently bound to a binder, either directly or through a first linker, said binder comprises affinity to said extracellular binding domain, and
c. incubating the cell of (b) with a second compound according to this invention, comprising a second oligonucleotide (ODN-2) covalently bound to a protein binder, either directly or through a second linker, wherein said second oligonucleotide is complementary to said first oligonucleotide, and said protein binder is selective to said POI, and
d. incubating said cell with said POI,
   thereby binding said cell to said POI.

In some embodiments, this invention relates to a kit comprising:
a. a recombinant cell ectopically expressing a polypeptide according to this invention, wherein said polypeptide comprises a membranal anchoring domain and an extracellular binding domain, said extracellular binding domain bound to
b. a first compound according to this invention, comprising a first oligonucleotide (ODN-1) covalently bound to a binder according to this invention, either directly or through a first linker, said binder comprises affinity to said extracellular binding domain,
c. a second compound according to this invention, comprising a second oligonucleotide (ODN-2) covalently bound to a synthetic agent, either directly or through a second linker, wherein said second oligonucleotide is complementary to said first oligonucleotide

In some embodiments, the polypeptide of the system, kit, recombinant cell and methods of the invention, is a cell surface protein (CSP). In some embodiments, the system does not perturb said cell's function, the system can be reversibly modified, or combination thereof. In some embodiments, the recombinant cell of the system, kit, recombinant cell and methods of the invention, is selected from: eukaryotes, prokaryotes, mammalian cells, plant cells, human cells, and bacteria. In some embodiments, the bacteria comprise E. coli. In some embodiments, the membranal anchoring domain of the system, kit, recombinant cell and methods of the invention, comprises a transmembranal protein or a part of it, an artificial polypeptide, or a combination thereof. In some embodiments, the transmembranal protein of the system, kit recombinant cell and methods of the invention, comprises an outer membrane protein C (OmpC); receptor tyrosine kinases (RTKs); Ion channel linked receptors; Enzyme-linked receptors; G protein-coupled receptors or any combination thereof. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder of the system, kit, artificial receptor, recombinant cell and methods of the invention, comprises a His-tag specific binder. In some embodiments, the His-tag specific binder comprises a three nitrilo acetic acid (Tri-NTA) group. In some embodiments, the His-tag specific binder comprises a moiety represented by the structure of formula E, E(a), E(b) as described herein below. In some embodiments, the first linker of the system, kit, artificial receptor, recombinant cell and methods of the invention, comprises at least one polyethyleneglycol (PEG) moiety, at least one phosphate moiety, at least one thioalkyl moiety or any combination thereof. In some embodiments, the first linker is represented by the following formula —[(CH₂O)ₖ—PO₃H]ₗ—(CH₂)_{w}—S— as described herein below. In some embodiments, the first compound of the system, kit, artificial receptor, recombinant cell and methods of the invention, further comprises a labeling moiety. In some embodiments, the labeling moiety is bound to the 3′ end or to the 5′ end of said first oligonucleotide, directly or through a third linker. In some embodiments, the fluorescent dye is selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof. In some embodiments, the first compound of the system, kit, artificial receptor, recombinant cell and methods of the invention, is represented by the structure of the nickel complexes of compounds 100-104 as described hereinbelow. In some embodiments, the second oligonucleotide of the system, kit, artificial receptor, recombinant cell and methods of the invention, is longer than said first oligonucleotide, said second oligonucleotide comprises a toehold region, or a combination thereof. In some embodiments, the synthetic agent of the second compound of the system, kit, artificial receptor, recombinant cell and methods of the invention, is bound to the 3′ end or to the 5′ end of said second oligonucleotide. In some embodiments, the synthetic agent of said second compound of the system, kit, artificial receptor, recombinant cell and methods of the invention, comprises a molecular marker, a labeling moiety, a fluorescent dye, an adhesion molecule, a cancer cell binder, a protein binder, a protein ligand, an anticancer agent, a surface binder, a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof. In some embodiments, the dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof; said protein binder comprises a biotin or a folate; said adhesion molecule comprises a folate; said surface binder is an abiotic surface binder; said surface binder comprises a thiol group (HS), a Si-halogen group, a Si—O bond; said cancer cell binder comprises a folate or any combination thereof. In some embodiments, the second compound of the system, kit, artificial receptor, recombinant cell and methods of the invention, further comprises a second labeling moiety. In some embodiments, the second labeling moiety is bound to the 3′ end or to the 5′ end of said second oligonucleotide, directly or through a fourth linker. In some embodiments, the second labeling moiety comprises a fluorescent dye. In some embodiments, the second compound of the system, kit, artificial receptor, recombinant cell and methods of the invention, is represented by the structure of compounds 200-207 as described hereinbelow. In some embodiments, the system or the kit of the invention further comprises a third compound comprising a third oligonucleotide (ODN-3), wherein said third oligonucleotide is complementary to said second oligonucleotide. In some embodiments, the third oligonucleotide is fully complementary to said second oligonucleotide. In some embodiments, the third oligonucleotide comprises higher affinity to said second oligonucleotide than the affinity of said second oligonucleotide to said first oligonucleotide.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

The patent of application file contains at least one drawing executed in color. Copies of this patent or patent application publication with color drawings will be provided by the office upon request and payment of the necessary fee.

FIGS. 1A-1B show the design of an artificial receptors system. FIG. 1A shows an embodiment to decorate E. coli with artificial receptors appended with a specific functionality (X). A first molecule X-ODN-1 binds a hexa-histidine tag (His-tag) fused to recombinant OmpC. Recombinant OmpC is inserted into the cell membrane. Reversibility of this process is achieved by subjecting the bacteria to EDTA. A further way to introduce an unnatural recognition motif (Y) to the bacterial surface is adding to the bacteria-bound ODN-1 an ODN-1 complementary strand modified with the desired functionality (Y-ODN-2). Y-ODN-2 can be selectively removed by adding a complementary strand, ODN-3. FIG. 1B shows the structure of X-ODN-1.

FIGS. 2A-2E shows reversible, non-covalent modification of bacterial membrane with a synthetic receptor. FIG. 2A shows fluorescence images of: (i) E. coli expressing His-OmpC incubated with 500 nM of Compound 100 and Ni (II), (ii) Native bacteria (that lack His-tag) incubated with 500 nM of Compound 100 and Ni (II), (iii) E. coli expressing His-OmpC incubated with 500 nM of Compound 100 in the absence of Ni (II), and (iv) E. coli expressing His-OmpC incubated with 500 nM of Cy5-ODN (that lacks the NTA group) and Ni (II). FIG. 2B shows flow cytometry analysis of His-tagged bacteria (yellow) and native bacteria (gray) incubated with Compound 101. FIG. 2C shows fluorescence images of E. coli expressing His-OmpC decorated with Compound 100 in the presence of increasing concentrations of EDTA (0, 5, and 10 mM) (left), and following subsequent addition of Compound 100 in the absence of Ni (II) (right). FIG. 2D shows the growth curve of E. coli expressing His-OmpC (black) and of the same bacteria decorated with Compound 101 (red). FIG. 2E shows bright field (top) and fluorescence images (bottom) of bacteria decorated Compound 101 monitored at 0, 12, and 24 hours.

FIGS. 3A-3B show the reversible modification of membrane-bound synthetic receptor using complementary strands. FIG. 3A shows a schematic illustration of the methods used in the experiment. His-tagged bacteria were sequentially modified by attaching them with ectopic molecules. First, cells were attached with a compound comprising TAMRA. Then, TAMRA was removed by incubating the cells with ODN-3. Then, cells were attached with a compound comprising Cy5. Then Cy5 was detached by incubating the cells with ODN-3. Then, cells were attached with a compound comprising FAM. Then, cells were attached with Cy5. Then Cy5 was detached by incubating the cells with ODN-3. FIG. 3B shows microscopic images of the emissions of TAMRA, Cy5, and FAM using 590 nm, 700/775 nm, and 510/550 nm emission filters, respectively.

FIGS. 4A-4D show experimental modifications of bacterial cell surface luminescence. FIG. 4A shows a schematic illustration of the experiment. (i) Different sub-populations of His-tagged cells were incubated with three types of ODN-1: Compound 102, Compound 103, and Compound 104. (ii) cells were incubated with three types of ODN-2: Compound 202, Compound 203, and Compound 204, complementary to Compound 102, Compound 103, and Compound 104, respectively. Compound 202, Compound 203, and Compound 204 were appended with FAM, TAMRA, and CY5, respectively. FIG. 4B shows a fluorescence overlay image of the labeled mixed population. Bacteria were imaged using 488 nm, 561 nm, and 647 nm excitation lasers and 488/50, 610/60, and 685/50 emission filters. FIG. 4C shows percentages of each sub-population counted and averaged from six different frames. FIG. 4D shows a flow cytometry analysis of the mixed population.

FIGS. 5A-5G show bacteria decorated to interact with proteins expressed by cancer cells. FIG. 5A shows a schematic illustration of an experiment in which modified His-tagged bacteria were treated with Alexa 647-modified streptavidin (Alexa-SA). Left: Bacteria were modified with a duplex generated from ODN-1 and Compound 205. Right: Bacteria were modified with a duplex lacking biotin. FIG. 5B shows Alexa-SA fluorescence in the cells incubated with Alexa 647-modified streptavidin. FIG. 5C shows images recorded following the incubation of the bacteria bound to Alexa-SA with ODN-3. FIG. 5D shows a schematic illustration of an experiment in which decorated bacteria were incubated with KB-cells. Left: Bacteria decorated with a duplex consisting of ODN-1 and TAMRA-labeled Compound 206. Right: Bacteria decorated with a duplex that lacks the folate group. FIG. 5E shows TAMRA-labeling of KB cells incubated with bacteria decorated with folate. FIG. 5F shows fluorescent images obtained after treating the bacteria that are bound to KB cells with ODN-3. FIG. 5G shows that incubating a KB-cell with a duplex consisting of ODN-1 and TAMRA-folate-ODN-2 (Compound 206), in the absence of bacteria, did not lead to fluorescent KB-cell labeling.

FIGS. 6A-6B shows bacteria decorated to interact to a non-biological surface. FIG. 6A shows microscopic images of: (i) bear gold substrate after incubation with unmodified bacteria, (ii) passivated gold substrate after incubation with unmodified bacteria, and (iii) passivated gold substrate following incubation with bacteria modified with a thiol-modified duplex (ODN-1:Compound 207). FIG. 6B shows the average bacteria count on passivated gold surfaces, which corresponds to an image area of ˜0.0165 mm².

FIGS. 7A-7B show super-resolution images of His-tagged bacteria decorated with an ODN-1:Compound 201 duplex. FIG. 7A shows whole bacteria. FIG. 7B shows a transverse cut viewed from the plane of the cell axis.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### System for Decorating Cell Membranes

In some embodiments, disclosed herein is a system comprising:

a. a recombinant cell ectopically expressing a polypeptide, wherein said polypeptide comprises a membranal anchoring domain and an extracellular binding domain,
b. a first compound comprising a first oligonucleotide (ODN-1) covalently bound to a binder, either directly or through a first linker, said binder comprising affinity to said extracellular binding domain,
c. a second compound comprising a second oligonucleotide (ODN-2) covalently bound to a synthetic agent, either directly or through a second linker, wherein said second oligonucleotide is complementary to said first oligonucleotide.

In some embodiments, the polypeptide is bound to the first compound, the second compound is bound to the first compound, or combination thereof, each represent a separate embodiment according to the invention. In some embodiments, when incubated together, the polypeptide, the first compound, and the second compound, form a complex, in which the polypeptide is attached to the first compound and the first compound is attached to the second compound. In some embodiments, the first compound is attached to the second compound via the hybridization of the first oligonucleotide to the second oligonucleotide. In some embodiments, the first compound is attached to the polypeptide via coordination of said binder to said extracellular binding domain of said polypeptide. In some embodiments, the first compound is attached to the polypeptide via coordination of said binder to an affinity tag comprised in said extracellular binding domain of said polypeptide. In some embodiments, the polypeptide is a cell surface proteins (CSPs). In some embodiments, the polypeptide is an outer membrane protein C (OmpC). In some embodiments, the polypeptide is a receptor tyrosine kinase (RTK).

In some embodiments, the system does not perturb said cell's function. In some embodiments, the system can be reversibly modified. In some embodiments, the recombinant cell is selected from: eukaryotes, prokaryotes, mammalian cells, plant cells, human cells, and bacteria. In some embodiments, the bacteria comprise E. coli. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(a), D(b), E, E(a), E(b), G, G(a), or G(b). In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof; each represents a separate embodiment according to this invention. In some embodiments, the second compound further comprises a second labeling moiety. In some embodiments, the second labeling moiety comprises a fluorescent dye. In some embodiments, the system further comprises a third compound comprising a third oligonucleotide (ODN-3), wherein said third oligonucleotide is complementary to said second oligonucleotide. In some embodiments, the third oligonucleotide comprises higher affinity to said second oligonucleotide than the affinity of said second oligonucleotide to said first oligonucleotide.

### A Kit for Decorating Cell Membranes

In some embodiments, this invention relates to a kit comprising:
a. a recombinant cell ectopically expressing a polypeptide according to this invention, wherein said polypeptide comprises a membranal anchoring domain and an extracellular binding domain, said extracellular binding domain bound to
b. a first compound according to this invention, comprising a first oligonucleotide (ODN-1) covalently bound to a binder according to this invention, either directly or through a first linker, said binder comprises affinity to said extracellular binding domain, and
c. a second compound according to this invention, comprising a second oligonucleotide (ODN-2) covalently bound to a synthetic agent, either directly or through a second linker, wherein said second oligonucleotide is complementary to said first oligonucleotide

In some embodiments, the polypeptide is bound to the first compound, the second compound is bound to the first compound, or combination thereof, each represent a separate embodiment according to the invention. In some embodiments, when incubated together, the polypeptide, the first compound, and the second compound, form a complex, in which the polypeptide is attached to the first compound and the first compound is attached to the second compound. In some embodiments, the complex can be reversibly modified. In some embodiments, the first compound is attached to the second compound via the hybridization of the first oligonucleotide to the second oligonucleotide. In some embodiments, the first compound is attached to the polypeptide via coordination of said binder to said extracellular binding domain of said polypeptide. In some embodiments, the first compound is attached to the polypeptide via coordination of said binder to an affinity tag comprised in said extracellular binding domain of said polypeptide. In some embodiments, the polypeptide is a cell surface proteins (CSPs). In some embodiments, the polypeptide is an outer membrane protein C (OmpC). In some embodiments, the polypeptide is a receptor tyrosine kinase (RTK). In some embodiments, the polypeptide is an ion channel linked receptor. In some embodiments, the polypeptide is an enzyme-linked receptor. In some embodiments, the polypeptide is a G protein-coupled receptor.

In some embodiments, the kit further comprises a third compound comprising a third oligonucleotide (ODN-3), wherein said third oligonucleotide is complementary to said second oligonucleotide. In some embodiments, the third oligonucleotide comprises higher affinity to said second oligonucleotide than the affinity of said second oligonucleotide to said first oligonucleotide. In some embodiments, the recombinant cell is selected from: eukaryotes, prokaryotes, mammalian cells, plant cells, human cells, and bacteria. In some embodiments, the bacteria comprise E. coli. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(a), D(b), E, E(a), E(b), G, G(a), or G(b). In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof; each represents a separate embodiment according to this invention. In some embodiments, the second compound further comprises a second labeling moiety. In some embodiments, the second labeling moiety comprises a fluorescent dye.

### Artificial Receptor

In some embodiments, disclosed herein is an artificial receptor, capable of binding a His-tagged protein, comprising:
a. a first compound comprising a first oligonucleotide (ODN-1) bound to a His-tag binder, either directly or through a first linker, said His-tag binder comprises a moiety represented by the structure of formula E:

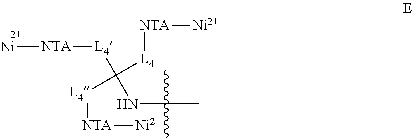

wherein

L₄, L₄′, and L₄″ is each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof; and
b. a second compound comprising a second oligonucleotide (ODN-2) covalently bound to a synthetic agent, either directly or through a second linker, said second oligonucleotide is complementary to said first oligonucleotide.

In some embodiments, the artificial receptor does not perturb the function of a living cell. In some embodiments, the receptor can be reversibly modified. In some embodiments, the binder comprises a moiety represented by the structure of formula C, D, D(a), D(b), E(a), E(b), G, G(a), or G(b) as described herein below; each represents a separate embodiment according to this invention. In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof; each represents a separate embodiment according to this invention. In some embodiments, the second compound further comprises a second labeling moiety. In some embodiments, the second labeling moiety comprises a fluorescent dye. In some embodiments, the artificial receptor further comprises a third compound comprising a third oligonucleotide (ODN-3), wherein said third oligonucleotide is complementary to said second oligonucleotide. In some embodiments, the third oligonucleotide comprises higher affinity to said second oligonucleotide than the affinity of said second oligonucleotide to said first oligonucleotide.

In some embodiments, the first compound is further attached to a polypeptide comprising a His-tag affinity tag, via the binding of said His-tag binder of the first compound, to the His-tag affinity tag of the polypeptide.

In some embodiments, the second compound is bound to the first compound. In some embodiments, when incubated together, the first compound, and the second compound, form a double helix complex, in which the first oligonucleotide is bound to the second oligonucleotide.

In some embodiments, a complex comprising the polypeptide, the first compound, and the second compound, wherein the polypeptide is attached to the first compound and the first compound is attached to the second compound, is termed herein an “artificial receptor”, “synthetic receptor”, “artificial receptor system”, or “synthetic receptor system”. In some embodiments, expressing a polypeptide in a cell and attaching to it a first compound, and in some embodiments, a second compound, is termed “decorating” a cell. In some embodiments, the terms “decorating”, “modifying” and “coating” are used herein interchangeably, having all the same meanings.

### Recombinant Cells

In some embodiments, disclosed herein is a recombinant cell ectopically expressing a polypeptide, wherein said polypeptide comprises a membranal anchoring domain and an extracellular binding domain, said extracellular binding domain bound to

a. a first compound comprising a first oligonucleotide (ODN-1) covalently bound to a binder, either directly or through a first linker, said binder comprising affinity to said extracellular binding domain,
b. a second compound comprising a second oligonucleotide (ODN-2) covalently bound to a synthetic agent, either directly or through a second linker, wherein said second oligonucleotide is complementary to said first oligonucleotide.

In some embodiments, the recombinant cell is selected from a group comprising eukaryotes, prokaryotes, mammalian cells, plant cells, human cells, and bacteria. In some embodiments, a mammalian or a human cell is selected from a group comprising epithelial cells, Brunner's gland cells in duodenum, insulated goblet cells of respiratory and digestive tracts, stomach, foveolar cells, chief cells, parietal cells, pancreatic acinar cells, Paneth cells of small intestine, Type II pneumocyte of lung, club cells of lung, barrier cells, type i pneumocytes, gall bladder epithelial cells, centroacinar cells, intercalated duct cells, intestinal brush border cells, hormone-secreting cells, enteroendocrine cells, K cells, L cells, I cells, G cells, enterochromaffin cells, enterochromaffin-like cells, N cells, S cells, D cells, Mo cells, thyroid gland cells, thyroid epithelial cells, parafollicular cells, parathyroid gland cells, parathyroid chief cells, oxyphil cells, pancreatic islets, alpha cells, beta cells, delta cells, epsilon cells, PP cells, salivary gland mucous cells, salivary gland serous cells, Von Ebner's gland cells in tongue, mammary gland cells, lacrimal gland cells, ceruminous gland cells in ear, eccrine sweat gland dark cells, eccrine sweat gland clear cells, apocrine sweat gland cells, gland of moll cells in eyelid, sebaceous gland cells, Bowman's gland cells in nose, hormone-secreting cells, anterior/intermediate pituitary cells, corticotropes, gonadotropes, lactotropes, melanotropes, somatotropes, thyrotropes, magnocellsular neurosecretory cells, parvocellsular neurosecretory cells, chromaffin cells, keratinocytes, epidermal basal cells, melanocytes, trichocytes, medullary hair shaft cells, cortical hair shaft cells, cuticular hair shaft cells, huxley's layer hair root sheath cells, Henle's layer hair root sheath cells, outer root sheath hair cells, surface epithelial cells of cornea, tongue, mouth, nasal cavity, distal anal canal, distal urethra, and distal vagina, basal cells, intercalated duct cells, striated duct cells, lactiferous duct cells, ameloblast, auditory inner hair cells of organ of Corti, auditory outer hair cells of organ of Corti, basal cells of olfactory epithelium, primary sensory neurons, Merkel cells of epidermis, olfactory receptor neuron, pain-sensitive primary sensory neurons, photoreceptor cells of retina in eye, proprioceptive primary sensory neurons, touch-sensitive primary sensory neurons, chemoreceptor glomus cells of carotid body cells, outer hair cells of vestibular system of ear, inner hair cells of vestibular system of ear, taste receptor cells of taste bud, neuron cells, interneurons, basket cells, cartwheel cells, Stellate cells, Golgi cells, granule cells, Lugaro cells, unipolar brush cells, Martinotti cells, chandelier cells, Cajal-Retzius cells, double-bouquet cells, neurogliaform cells, retina horizontal cells, amacrine cells, spinal interneuron, renshaw cells, spindle neurons, fork neurons, pyramidal cells, place cells, grid cells, speed cells, head direction cells, Betz cells, stellate cells, boundary cells, bushy cells, Purkinje cells, medium spiny neurons, astrocytes, oligodendrocytes, ependymal cells, tanycytes, pituicytes, adipocytes, white fat cells, brown fat cells, liver lipocytes, cells of the adrenal cortex, cells of the zona glomerulosa, cells of the zona fasciculata, cells of the zona reticularis, theca interna cells of ovarian follicle, granulosa lutein cells, theca lutein cells, leydig cells of testes, seminal vesicle cells, prostate gland cells, bulbourethral gland cells, Bartholin's gland cells, gland of littre cells, uterus endometrium cells, juxtaglomerular cells, macula densa cells of kidney, peripolar cells of kidney, mesangial cells of kidney, parietal epithelial cells, podocytes, proximal tubule brush border cells, loop of Henle thin segment cells, kidney distal tubule cells, kidney collecting duct cells, principal cells, intercalated cells, transitional epithelium, duct cells, efferent ducts cells, epididymal principal cells, epididymal basal cells, endothelial cells, planum semilunatum epithelial cells of vestibular system of ear, organ of Corti interdental epithelial cells, loose connective tissue fibroblasts, corneal fibroblasts, tendon fibroblasts, bone marrow reticular tissue fibroblasts, other nonepithelial fibroblasts, pericytes, hepatic stellate cells, nucleus pulposus cells of intervertebral disc, hyaline cartilage chondrocytes, fibrocartilage chondrocytes, elastic cartilage chondrocytes, osteoblast/osteocytes, osteoprogenitor cells, hyalocyte of vitreous body of eye, stellate cells of perilymphatic space of ear, pancreatic stellate cells, red skeletal muscle cells, white skeletal muscle cells, intermediate skeletal muscle cells, nuclear bag cells of muscle spindle, nuclear chain cells of muscle spindle, myosatellite cells, cardiac muscle cells, cardiac muscle cells, node cells, Purkinje fiber cells, smooth muscle cells, myoepithelial cells of iris, myoepithelial cells of exocrine glands, erythrocytes, megakaryocytes, platelets, monocytes, connective tissue macrophage, epidermal Langerhans cells, osteoclast, dendritic cells, microglial cells, neutrophil granulocytes, eosinophil granulocytes, basophil granulocytes, hybridoma cells, mast cells, helper T cells, suppressor T cells, cytotoxic T cells, natural killer T cells, B cells, natural killer cells, reticulocytes, hematopoietic stem cells and committed progenitors for the blood and immune system, oogonium/oocytes, spermatids, spermatocytes, spermatogonium cells, spermatozoon, and interstitial kidney cells.

In some embodiments, a prokaryote comprises a microbial cell such as bacteria, e.g., Gram-positive or Gram-negative bacteria. In some embodiments, the bacteria comprise Gram-negative bacteria or Negativicutes that stain negative in Gram stain. In some embodiments, the bacteria comprise gram-positive bacteria, gram-negative bacteria, or archaea.

In some embodiments, Gram-negative bacteria comprise Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chlamydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica, Yersinia pestis.

In some embodiments, the bacteria comprise gammaproteobacteria (e.g. Escherichia coli, pseudomonas, vibrio and klebsiella) or Firmicutes (belonging to class Negativicutes that stain negative in Gram stain).

In some embodiments, Gram-positive bacteria comprise Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Propionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

In some embodiments the bacteria is a species selected from the group consisting of Escherichia, Shigella, Salmonella, Erwinia, Yersinia, Bacillus, Vibrio, Legionella, Pseudomonas, Neisseria, Bordetella, Helicobacter, Listeria, Agrobacterium, Staphylococcus, Streptococcus, Enterococcus, Clostridium, Corynebacterium, Mycobacterium, Treponema, Borrelia, Francisella, Brucella, Campylobacter, Klebsiella, Frankia, Bartonella, Rickettsia, Shewanella, Serratia, Enterobacter, Proteus, Providencia, Brochothrix, and Brevibacterium.

In some embodiments, an oligonucleotide encoding the polypeptide is incorporated in an expression vector. In some embodiments, an oligonucleotide encoding the polypeptide is incorporated in a viral vector. An expression or viral vector can be introduced to the cell by any of the following: transfection, electroporation, infection, or transduction. In other embodiments, the polypeptide is encoded by an mRNA polynucleotide which is delivered for example by electroporation. In one embodiment, methods of electroporation comprise flow electroporation technology.

A skilled artisan would appreciate that the term “vector” encompasses a nucleic acid molecule capable of transporting another nucleic acid to which it has been linked. One type of vector is a “plasmid”, which encompasses a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. A skilled artisan would appreciate that the terms “plasmid” and “vector” may be used interchangeably having all the same qualities and meanings. In one embodiment, the term “plasmid” is the most commonly used form of vector. However, the disclosure presented herein is intended to include such other forms of expression vectors, such as viral vectors (e.g., replication defective retroviruses, lentivirus, adenoviruses and adeno-associated viruses), which serve equivalent functions. Additionally, some viral vectors are capable of targeting a particular cell type either specifically or non-specifically.

The recombinant expression vectors disclosed herein comprise a nucleic acid in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, a skilled artisan would appreciate that the term “operably linked” may encompass nucleotide sequences of interest linked to the regulatory sequence(s) in a manner that allows for expression of the nucleotide sequence (e.g., in an in vitro transcription/translation system or in a host cell when the vector is introduced into the host cell). A skilled artisan would appreciate that term “regulatory sequence” may encompass promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors disclosed here may be introduced into host cells to thereby produce proteins or peptides, including fusion proteins or peptides, encoded by nucleic acids as described herein. For example, an expression vector comprises a nucleic acid encoding a polypeptide comprising a membranal anchoring domain and an extracellular binding domain.

Another embodiment disclosed herein pertains to host cells into which a recombinant expression vector disclosed here has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

For stable transfection of mammalian cells, it is known that, depending upon the expression vector and transfection technique used, only a small fraction of cells may integrate the foreign DNA into their genome the remainder of the DNA remains episomal. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the polypeptide or can be introduced on a separate vector. Cells stably transfected with the introduced nucleic acid can be identified by drug selection (e.g., cells that have incorporated the selectable marker gene will survive, while the other cells die). In another embodiment the transfected cells are identified by the induction of expression of an endogenous reporter gene. In another embodiment the transfected cells are identified by the expression of the polypeptide.

A skilled artisan would appreciate that there are several methods in the art to identify recombinant cells expressing the polypeptide. In some embodiments, the expression of the mRNA encoding the polypeptide can be measured by RT-PCR. In some embodiments, the insertion of a DNA encoding the polypeptide can be identified by DNA gene sequencing. In some embodiments, expression of the polypeptide can be detected by an antibody, for example by Western blotting or ELISA. In some embodiments, the expression of a His-tag on the cell membrane can be detected by a labeled His-tag binder, for example by any of the binders disclosed herein, or by any other His-tag binder available.

In some embodiments, the cell's function is not disturbed by the presence of the polypeptide, the first, and the second compound on its surface. In some embodiments, the cell can be reversibly modified. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(a), D(b), E, E(a), E(b), G, G(a), or G(b), as described herein below; each is a separate embodiment. In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof; each represents a separate embodiment according to this invention. In some embodiments, the second compound further comprises a second labeling moiety. In some embodiments, the second labeling moiety comprises a fluorescent dye.

### Membranal Anchoring Domain

In some embodiments, the polypeptide according to this invention is a Cell Surface Protein (CSP). In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention.

In some embodiments, the polypeptide comprises a membranal anchoring domain. In some embodiments, a membranal anchoring domain comprises a polypeptide that, when expressed in a cell, it attaches to the cell membrane. In some embodiments, a membranal anchoring domain comprises at least one end emerging to the extracellular side. In some embodiments, the membranal anchoring domain comprises a transmembranal protein. In some embodiments, the membranal anchoring domain comprises a transmembranal fragment of a protein. In some embodiments, the protein comprises a protein expressed in the recombinant cell. In some embodiments, the protein comprises a cell not expressed in the recombinant cell. In some embodiments, the anchoring domain comprises an artificial polypeptide.

A skilled artisan would appreciate that a membrane anchoring can be selected to be stably expressed in the recombinant cell. For example, the membrane anchoring domain can comprise a protein that is abundantly expressed in the recombinant cell. In some embodiments, the membrane anchoring comprises a protein or a part of it, known to be abundantly expressed on the membrane of the recombinant cell. Thus, a membrane anchoring can be chosen to be a protein abundantly expressed on the recombinant cell membrane.

In some embodiments, a membrane anchoring comprises outer membrane protein C (OmpC) or a part thereof. In some embodiments, the transmembranal protein comprises an outer membrane protein C (OmpC); receptor tyrosine kinases (RTKs); Ion channel linked receptors; Enzyme-linked receptors; G protein-coupled receptors, a part thereof or any combination thereof; each represents a separate embodiment according to this invention. In some embodiments, a membrane anchoring comprises a polypeptide comprising at least 80% homology to any of SEQ ID NO.: 13, 16, or 21.

### Extracellular Binding Domain

In some embodiments, the extracellular domain comprised in the recombinant polypeptide comprises an affinity tag. In some embodiments, the binder comprises affinity to a specific affinity tag in the extracellular binding domain.

In some embodiments, an affinity tag comprises a protein tag. In some embodiments, an affinity tag comprises an epitope tag. In some embodiments, an affinity tag comprises a peptide tag. In some embodiments, an affinity tag comprises a combination of a number of tags.

In some embodiments, affinity tags are enzymatically modified, for example they are biotinylatated by biotin ligase. In some embodiments, affinity tags are chemically modified. In some embodiments, expression of a tag does not interfere with the cell functions. In some embodiments, an affinity tag can be removed by specific proteolysis. In some embodiments, tags are removed by TEV protease, Thrombin, Factor Xa or Enteropeptidase.

In some embodiments, an affinity tag is selected from a group comprising AviTag, C-tag, Calmodulin-tag, polyglutamate tag, E-tag, FLAG-tag, HA-tag, His-tag, 5-10 histidines bound by a nickel or cobalt chelate (H IHHH), Myc-tag, NE-tag, Rho1D4-tag, S-tag, SBP-tag, Softag 1, Softag 3, Spot-tag, Strep-tag, TC tag, Ty tag, V5 tag, VSV-tag, Xpress tag, Isopeptag, SpyTag, SnoopTag, SnoopTagJr, DogTag, SdyTag, BCCP (Biotin Carboxyl Carrier Protein), Glutathione-S-transferase-tag, Green fluorescent protein-tag, HaloTag, SNAP-tag, CLIP-tag, Maltose binding protein-tag, Nus-tag, Thioredoxin-tag, Fc-tag, Designed Intrinsically Disordered tags containing disorder promoting amino acids (P, E, S, T, A, Q, G, . . . ), and Carbohydrate Recognition Domain or CRDSAT-tag; each represents a separate embodiment.

In some embodiments, an affinity tag comprises a poly-histidine peptide comprising 6 histidine residues (6×-His-tag). In some embodiments, an affinity tag comprises a poly-histidine peptide comprising 10 histidine residues (10×-His-tag). In some embodiments, an affinity tag comprises a tetra cysteine peptide (CCPGCC, TC tag).

In some embodiments, more than one type of extracellular binding domain or affinity tag is used. A skilled artisan would recognize using more than one type of extracellular binding domain allows decorating the cell with more than one type of receptor. For example, a first extracellular binding domain and a second extracellular binding domain can be co-expressed in a recombinant cell. The recombinant cell is then incubated with a first and a second binder, wherein the first binder binds the first extracellular binding domain and the second binder binds the second extracellular binding domain. Thus, the first and the second binders will be bound to the same recombinant cell.

### The First Compound (X-ODN-1)

In some embodiments, the first compound, of the system, the artificial receptor, the recombinant cell, and the methods according to this invention (i.e., X-ODN-1) comprises:
a. a first oligonucleotide (ODN-1),
b. a binder which comprises affinity to a tagged polypeptide,
c. optionally a first linker which links the first oligonucleotide with the binder,
d. optionally a labeling moiety; and
e. optionally a third linker which links the first oligonucleotide with the labeling moiety.

In some embodiments, the first oligonucleotide is directly bound to the binder. In other embodiments, the first oligonucleotide is bound to the binder through a first linker. In some embodiments, the first oligonucleotide is directly bound to the labeling moiety. In other embodiments, the first oligonucleotide is bound to the labeling moiety through a third linker.

In some embodiments, the first compound of the system, the artificial receptor, the recombinant cell, and the methods according to this invention (i.e., X-ODN-1) is represented by the structure of formula J:

F-L₃-ODN1-L₁-Y₁ (J)

wherein
F is a labeling moiety (e.g., a dye or a dye derivative) or absent;
L₃ is a third linker or absent;
ODN1 is a first oligonucleotide sequence;
L₁ is a first linker or absent; and
Y₁ is a binder.

In some embodiments, the first compound of the system, the artificial receptor, the recombinant cell, and the methods according to this invention (i.e., X-ODN-1) is represented by the structure of formula H(a):

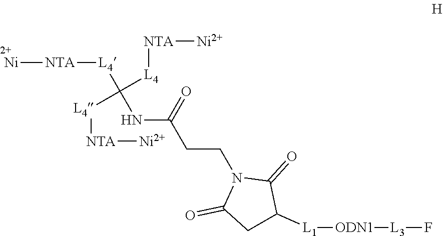

wherein
F is a labeling moiety or absent (e.g., a dye or a dye derivative);
L₃ is a third linker or absent;
ODN1 is a first oligonucleotide sequence;
L₁ is a first linker or absent;
L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms, or any combination thereof.

In some embodiments, the first compound of the system, the artificial receptor, the recombinant cell, and the methods according to this invention (i.e., X-ODN-1) is represented by the structure of formula H(a):

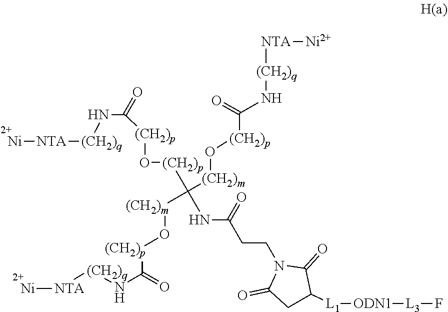

wherein
F is a labeling moiety or absent (e.g., a dye or a dye derivative);
L₃ is a third linker or absent;
ODN1 is a first oligonucleotide sequence;
L₁ is a first linker or absent;
m, p and q are each independently an integer number between 1 and 8.

In some embodiments, the first compound of the system, the artificial receptor, the recombinant cell, and the methods according to this invention (i.e., X-ODN-1) is represented by the structure of formula H(b):

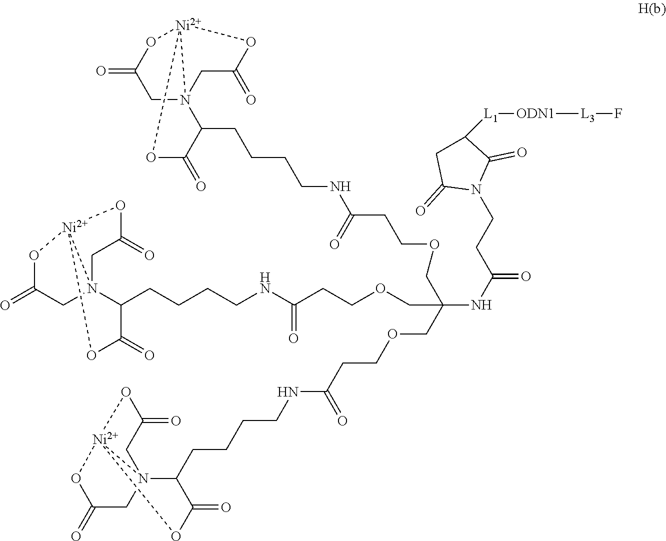

wherein
F is a labeling moiety or absent (e.g., a dye or a dye derivative);
L₃ is a third linker or absent;
ODN1 is a first oligonucleotide sequence; and
L₁ is a first linker or absent.

In some embodiments, the first compound of the system, the artificial receptor, the recombinant cell, and the methods according to this invention (i.e., X-ODN-1) is represented by the structure of the nickel complexes of the following compounds:

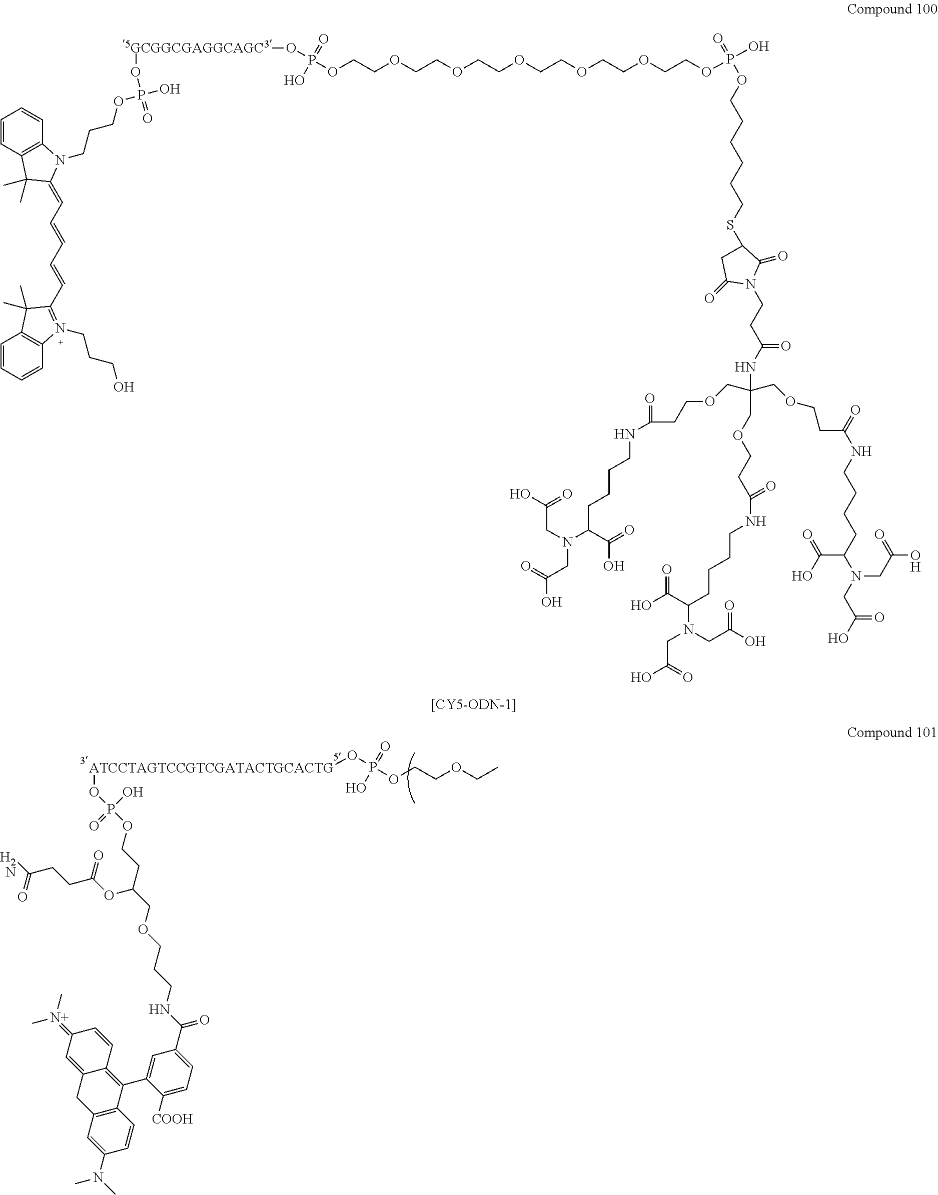

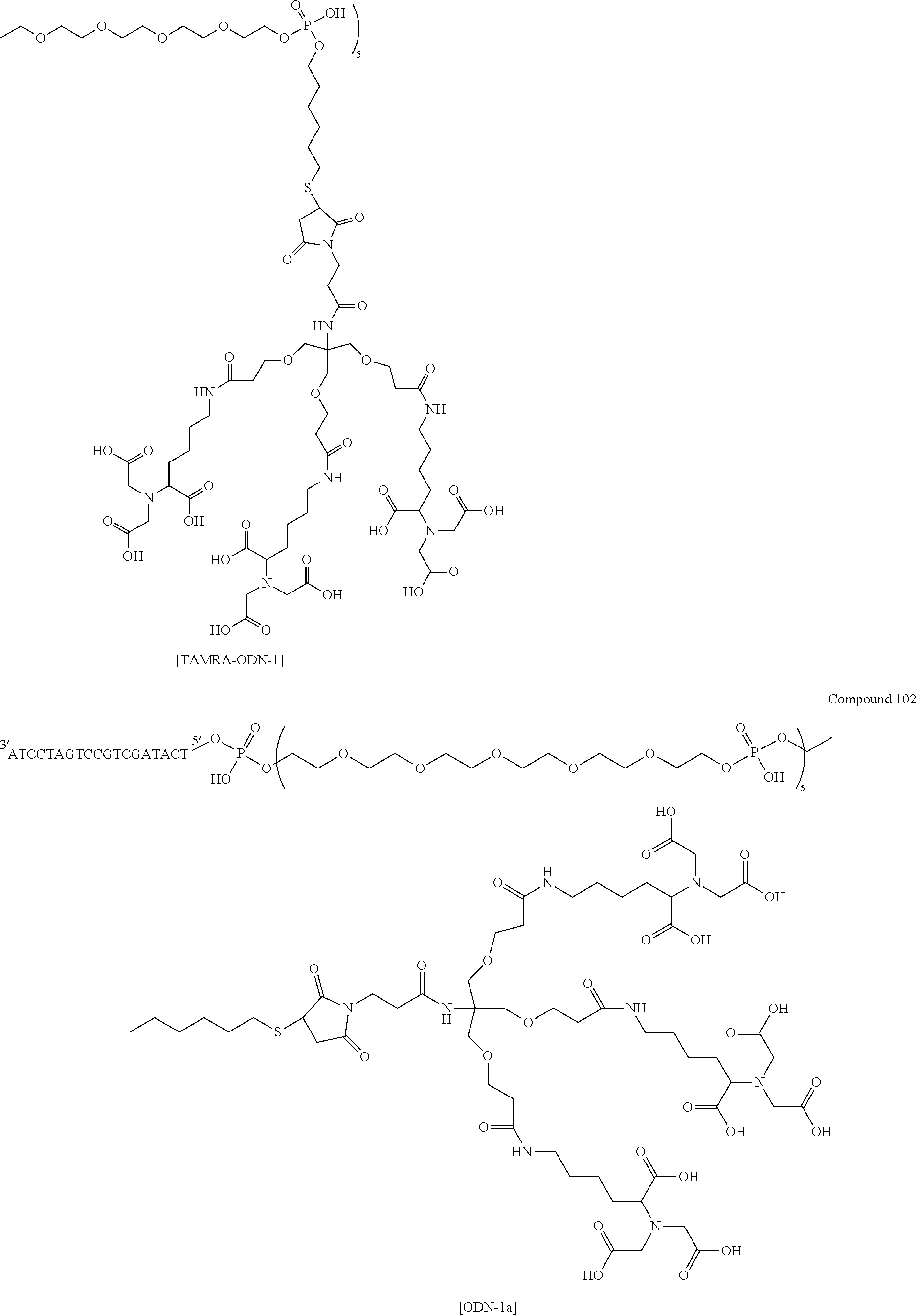

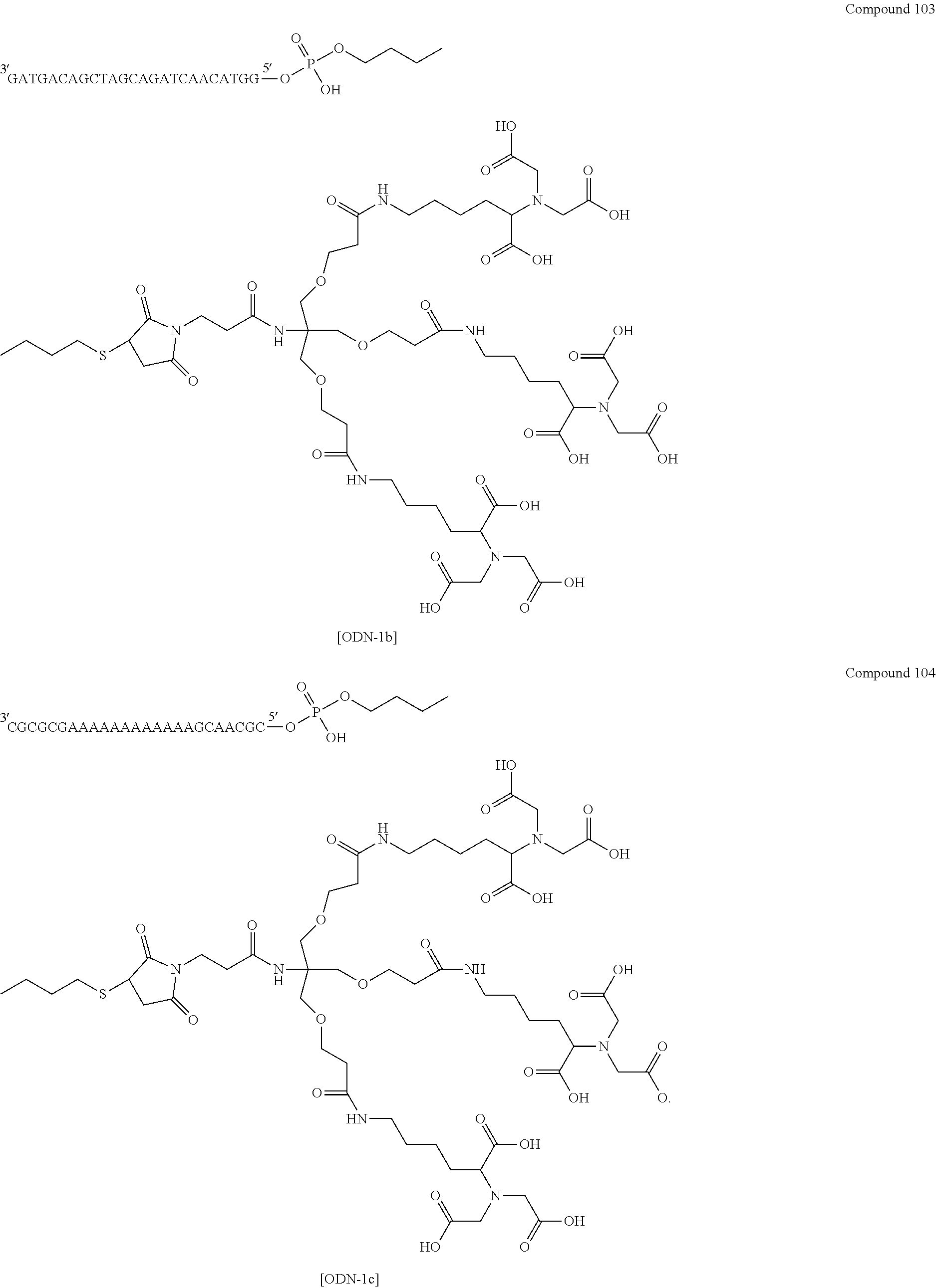

In some embodiments, Y₁ of formulas J is a binder. In some embodiments, Y₁ is an aptamer, a natural ligand, a synthetic group, or a peptide which binds a specific protein with high affinity and selectivity. In some embodiments, Y₁ comprises any selective protein binder known in the art. In another embodiment, Y₁ comprises marimastat, ethacrynic acid, bisethacrynic acid, complexed nitrilotriacetic acid (NTA), complexed bis NTA, complexed tris-NTA, Ni-nitrilotriacetic acid (Ni-NTA), bis Ni-NTA, tris-Ni-NTA, PDGF-BB, heparin, FGF aptamer, estrogen, DNA aptamer, RNA aptamer, peptide aldehyde, estrogen, suberoylanilidehydroxamic acid (SAHA), or a peptide binder. In another embodiment, the complexed NTA, complexed bis-NTA, complexed tris NTA is a nickel or cobalt complex. In some embodiments, Y₁ comprises a Tag-binding region. In some embodiments, Y₁ comprises any molecule that can target different type of affinity tags, such as poly-histidine peptide (HHHHHH, His-tag), or tetra cysteine peptide (CCPGCC, TC tag). In another embodiment, Y₁ comprises FlAsH probe. In another embodiment, Y₁ comprises ReAsH probe. In some embodiments, Y₁ comprises a His-tag binder. In some embodiments, Y₁ is a His-tag binder. In some embodiments, Y₁ comprises Ni-nitrilotriacetic acid (Ni-NTA), bis-Ni-NTA, or tris-Ni-NTA. In some embodiments, Y₁ comprises a derivative of Ni-nitrilotriacetic acid (Ni-NTA), bis-Ni-NTA, or tris-Ni-NTA, wherein the term “derivative” includes but not limited to alkyl derivatives, amide derivatives, amine derivatives, carboxy derivatives, and the like. In some embodiments, Y₁ comprises a derivative of tris-Ni-nitrilotriacetic acid (tris-Ni-NTA), a derivative of bis-Ni-nitrilotriacetic acid (bis-Ni-NTA), a derivative of mono-Ni-nitrilotriacetic acid (Ni-NTA); each represents a separate embodiment according to this invention. In some embodiments, Y₁ comprises any monomolecular compound which comprises three Ni-NTA moieties (i.e., tris-Ni-NTA). In some embodiments, Y₁ is represented by the structure of formulas D, D(a), D(b), G, G(a), G(b) as described herein below. In some embodiments, Y₁ comprises the structure of formulas D, D(a), D(b), G, G(a), G(b) as described herein below.

In some embodiments, L₁ of formulas J, H, H(a), and H(b) is a first linker. In some embodiments, L₁ is absent. In some embodiments, L₁ is bound to the 3′ end of ODN1. In some embodiments, L₁ is bound to the 5′ end of ODN1. In some embodiments, L₁ is bound to Y₁ through an amide bond, an ester bond, a phosphate bond, an ether bond, a thioether bond, each represents a separate embodiment according to this invention. In some embodiments, L₁ is as defined for the “first linker” hereinbelow.

In some embodiments, ODN1 of formulas J, H, H(a), and H(b) is a first oligonucleotide sequence. In some embodiments, ODN1 is directly bound to Y₁, through an amide bond, an ester bond, a phosphate bond, an ether bond, each represents a separate embodiment according to this invention. In some embodiments, ODN1 is directly bound to F, through an amide bond, an ester bond, a phosphate bond, an ether bond, a thioether bond, each represents a separate embodiment according to this invention. In some embodiments, ODN1 is directly bound to F, through a phosphate moiety.

In some embodiments, L₃ of formulas J, H, H(a), and H(b) is a third linker. In some embodiments, L₃ is absent. In some embodiments, L₃ is bound to the 3′ end of ODN1. In some embodiments, L₃ is bound to the 5′ end of ODN1. In some embodiments, L₃ is bound to F through an amide bond, an ester bond, a phosphate bond, an ether bond, a thioether bond, each represents a separate embodiment according to this invention. In some embodiments, L₃ is as defined for the “third linker” hereinbelow.

In some embodiments, F of formulas J, H, H(a), and H(b) is a labeling moiety. In some embodiments, F is absent. In some embodiments, F is a dye. Examples of dyes include but are not limited to: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof. In some embodiments, F is a dye derivative. In some embodiments, a labeling moiety is bound to ODN1 through an amide bond, an ester bond, a phosphate bond, an ether bond, a thioether bond; each represents a separate embodiment according to this invention. In some embodiments, a labeling moiety F is bound to L₃ through an amide bond, an ester bond, a phosphate bond, an ether bond, a thioether bond; each represents a separate embodiment according to this invention.

Linkers (L₁ and L₃)

In some embodiments, the first compound of the system, the artificial receptor, the recombinant cell, and the methods according to this invention (i.e., X-ODN-1) comprises:
a. a first oligonucleotide (ODN-1)
b. a binder which comprises affinity to the extracellular binding domain of said polypeptide,
c. optionally a first linker which links the first oligonucleotide with the binder
d. optionally a labeling moiety, and
e. optionally a third linker which links the first oligonucleotide with the labeling moiety.

The terms “linker” or “spacer” are used interchangeably, and refer to a chemical fragment that connects between the 5′ or the 3′ end of an oligonucleotide according to this invention, and other chemical moieties of the system of the invention (e.g., binder, labeling moiety or a dye, synthetic agent, etc). In some embodiments, the linker is covalently bound to the oligonucleotide through a phosphate moiety.

### A First Linker (L₁)

In some embodiments, the first compound (X-ODN-1) of the system, the artificial receptor, the recombinant cell, and the methods according to the invention, comprises a first linker, which links the first oligonucleotide with the binder. In some embodiments, the first linker is covalently bound to the 3′ end of the first oligonucleotide (ODN-1). In some embodiments, the first linker is covalently bound to the 5′ end of the first oligonucleotide. In some embodiments, the first linker is covalently bound to the binder through an amide bond, an ester bond, a phosphate bond, an ether bond, a thioether bond; each represents a separate embodiment according to this invention. In some embodiments, the first linker is covalently bound to the first oligonucleotide through an amide bond, an ester bond, a phosphate bond, an ether bond, a thioether bond, each represents a separate embodiment according to this invention. In some embodiments, the first linker is covalently bound to the first oligonucleotide through a phosphate moiety.

In some embodiments, the first linker of the system, the artificial receptor, the recombinant cell, and the methods, and/or L₁ according to formula J, H, H(a), and H(b) is any chemical fragment which comprises at least one segment of a commercially available phosphoramidite spacer derivative. Phosphoramidite compounds are used as reactive agents for linking oligonucleotides according to this invention with other moieties, e.g., the binder of this invention, the labeling moiety, the synthetic agents, etc. Non limiting examples of such phosphoramidite derivatives, useful for linking oligonucleotides with other moieties include:

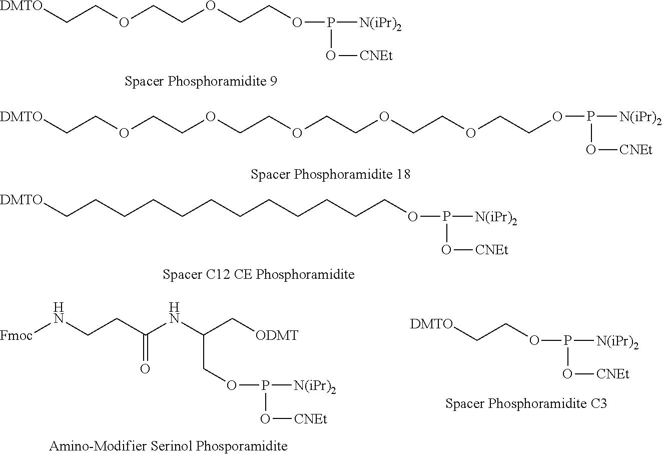

In some embodiments, the first linker of the system, the artificial receptor, the recombinant cell, and the methods, and/or L₁ according to formula J, H, H(a), and H(b) is a substituted or unsubstituted linear or branched alkyl chain of 1-20 carbon atoms, substituted or unsubstituted linear or branched alkyl ether chain of 1-20 carbon atoms, oligoethylene glycol, polyethylene glycol (PEG), oligopropylene glycol, polypropylene glycol (PPG), substituted or unsubstituted linear or branched thioalkyl chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl ester of 1-20 carbon atoms, substituted or unsubstituted linear or branched alkyl diamide chain of 2-10 carbon atoms, substituted or unsubstituted linear or branched alkyl triazole of 1-20 carbon atoms, substituted or unsubstituted linear or branched alkyl amine chain of 1-20 carbon atoms or any combination thereof; each represents a separate embodiment according to this invention.

In some embodiments, the first linker of the system, the artificial receptor, the recombinant cell, and the methods, and/or L₁ according to formula J, H, H(a), and H(b) comprises at least one polyethyleneglycol (PEG) moiety. In some embodiments, the first linker, and/or L₁ comprises at least one phosphate moiety. In some embodiments, the first linker, and/or L₁ comprises at least one alkyl ether moiety. In some embodiments, the first linker, and/or L₁ comprises at least one alkyl diamide moiety. In some embodiments, the first linker, and/or L₁ comprises at least one alkyl moiety. In some embodiments, the first linker, and/or L₁ comprises at least one thioalkyl moiety. In some embodiments, the first linker, and/or L₁ comprises at least one polyethyleneglycol (PEG) moiety, at least one phosphate moiety, at least one thioalkyl moiety, at least one alkyl moiety, or any combination thereof.

In some embodiments, the first linker of the system, the artificial receptor, the recombinant cell, and the methods, and/or L₁ according to formula J, H, H(a), and H(b) is represented by the following formula:

—[(CH₂O)ₖ—PO₃H]—(CH₂)_{w}—S—

wherein
k and l are each independently an integer number between 0 and 10; and
w is an integer number between 1 and 10.

In some embodiments, k is 0. In some embodiments, k is 6. In some embodiments, k is 1, 2, 3, 4, 5, 7, 8, 9, 10; each is a separate embodiment according to this invention.

In some embodiments, 1 is 0. In some embodiments, 1 is 1. In some embodiments, 1 is 5. In some embodiments, 1 is 2, 3, 4, 6, 7, 8, 9, 10; each is a separate embodiment according to this invention.

In some embodiments, w is 6. In some embodiments, w is 1, 2, 3, 4, 5, 7, 8, 9, 10; each is a separate embodiment according to this invention.

### A Third Linker (L₃)

In some embodiments, the first compound (X-ODN-1) of the system, the artificial receptor, the recombinant cell, and the methods comprises a third linker, which links the first oligonucleotide with the labeling moiety. In some embodiments, the third linker is absent. In some embodiments, the third linker is bound to the 3′ end of ODN-1. In some embodiments, the third linker is bound to the 5′ end of ODN-1. In some embodiments, the third linker is a part of a commercially available phosphoramidite dye derivative. In some embodiments, the third linker is bound to the labeling moiety through an amide bond, an ester bond, a phosphate bond, an ether bond, a thioether bond; each represents a separate embodiment according to this invention. In some embodiments, the third linker is bound to ODN-1 through an amide bond, an ester bond, a phosphate bond, an ether bond, a thioether bond; each represents a separate embodiment according to this invention. In some embodiments, the third linker is covalently bound to the first oligonucleotide through a phosphate moiety.

In some embodiments, the third linker of the system, the artificial receptor, the recombinant cell, and the methods and/or L₃ according to formula J, H, H(a), and H(b), is a substituted or unsubstituted linear or branched alkyl chain of 1-20 carbon atoms, substituted or unsubstituted linear or branched alkyl ether chain of 1-20 carbon atoms, oligoethylene glycol, polyethylene glycol (PEG), oligopropylene glycol, polypropylene glycol (PPG), substituted or unsubstituted linear or branched thioalkyl chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl ester of 1-20 carbon atoms, substituted or unsubstituted linear or branched alkyl diamide chain of 2-10 carbon atoms, substituted or unsubstituted linear or branched alkyl triazole of 1-20 carbon atoms, substituted or unsubstituted linear or branched alkyl amine chain of 1-20 carbon atoms or any combination thereof; each is a separate embodiment according to this invention.

In some embodiments, the third linker of the system, the artificial receptor, the recombinant cell, and the methods, and/or L₃ according to formula J, H, H(a), and H(b) comprises at least one polyethyleneglycol (PEG) moiety. In some embodiments, the third linker, and/or L₃ comprises at least one phosphate moiety. In some embodiments, the third linker, and/or L₃ comprises at least one alkyl ether moiety. In some embodiments, the third linker, and/or L₃ comprises at least one alkyl diamide moiety. In some embodiments, the third linker, and/or L₃ comprises at least one alkyl moiety. In some embodiments, the third linker, and/or L₃ comprises at least one thioalkyl moiety. In some embodiments, the third linker, and/or L₃ comprises at least one polyethyleneglycol (PEG) moiety, at least one phosphate moiety, at least one thioalkyl moiety, at least one alkyl moiety, or any combination thereof.

In some embodiments, the third linker of the system, the artificial receptor, the recombinant cell, and the methods, and/or L₃ according to formula J, H, H(a), and H(b) is represented by the following formula:

—[(CH₂O)ₖ—PO₃H]—(CH₂)_{w}—S—

wherein
k and l are each independently an integer number between 0 and 10; and
w is an integer number between 1 and 10.

In some embodiments, k is 0. In some embodiments, k is 6. In some embodiments, k is 1, 2, 3, 4, 5, 7, 8, 9, 10; each is a separate embodiment according to this invention.

In some embodiments, 1 is 0. In some embodiments, 1 is 1. In some embodiments, 1 is 5. In some embodiments, 1 is 2, 3, 4, 6, 7, 8, 9, 10; each is a separate embodiment according to this invention.

In some embodiments, w is 6. In some embodiments, w is 1, 2, 3, 4, 5, 7, 8, 9, 10; each is a separate embodiment according to this invention.

### Binder (Y₁)

In some embodiments, a binder of the system, the artificial receptor, the recombinant cell, and the methods according to this invention is an aptamer, a natural ligand, a synthetic group, or a peptide, which binds a specific protein with high affinity and selectivity.

In some embodiments, the binder of the system, the artificial receptor, the recombinant cell, and the methods of this invention is any selective protein binder known in the art. In another embodiment, the selective protein binder comprises marimastat, ethacrynic acid, bisethacrynic acid, complexed nitrilotriacetic acid (NTA), complexed bis NTA, complexed tris-NTA, Ni-nitrilotriacetic acid (Ni-NTA), bis Ni-NTA, tris-Ni-NTA, PDGF-BB, heparin, FGF aptamer, estrogen, DNA aptamer, RNA aptamer, peptide aldehyde, estrogen, suberoylanilidehydroxamic acid (SAHA), or a peptide binder. In another embodiment, the complexed NTA, complexed bis-NTA, complexed tris NTA is a nickel or cobalt complex.

In some embodiments, the binder comprises a Tag-binding region.

In some embodiments, the binder is any molecule that can target different type of affinity tags, such as poly-histidine peptide (HHHHHH, His-tag), or tetra cysteine peptide (CCPGCC, TC tag). In another embodiment, the binder is FlAsH probe. In another embodiment, the binder is ReAsH probe.

In some embodiments, the selective binder is a His-tag binder. In some embodiments, the binder of this invention comprises Ni-nitrilotriacetic acid (Ni-NTA), bis-Ni-NTA, or tris-Ni-NTA. In some embodiments, the binder of this invention comprises a derivative of Ni-nitrilotriacetic acid (Ni-NTA), bis-Ni-NTA, or tris-Ni-NTA, wherein the term “derivative” includes but not limited to alkyl derivatives, amide derivatives, amine derivatives, carboxy derivatives, and the like In some embodiments, the His-Tag binder comprises a derivative of tris-Ni-nitrilotriacetic acid (tris-Ni-NTA), a derivative of bis-Ni-nitrilotriacetic acid (bis-Ni-NTA), a derivative of mono-Ni-nitrilotriacetic acid (Ni-NTA); each represents a separate embodiment according to this invention. In some embodiments, the His-tag binder is any monomolecular compound which comprises three Ni-NTA moieties (i.e., tris-Ni-NTA).

In some embodiments, the binder according to this invention is a His-tag binder.

In some embodiments, the His-tag binder comprised in the system, the artificial receptor, the recombinant cell, and the methods of the invention comprises a moiety represented by the structure of Formula C:

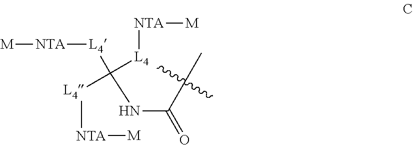

wherein
L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof; and M-NTA is a metal complex of nitrilotriacetic acid.

In some embodiments, M is a metal ion. In some embodiments, M is cobalt (Co). In some embodiments, M is nickel (Ni). In some embodiments, M is Ni(II). In some embodiments, M is Co(II). In some embodiments, M is Co(III).

In some embodiments, the His-tag binder comprised in the system, the artificial receptor, the recombinant cell, and the methods of the invention comprises a moiety represented by the structure of formula D:

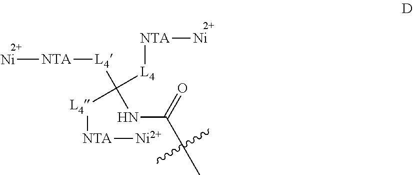

wherein
L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In another embodiment, the His-tag binder comprised in the system, the artificial receptor, the recombinant cell, and the methods of the invention comprises a moiety represented by the structure of formula D(a):

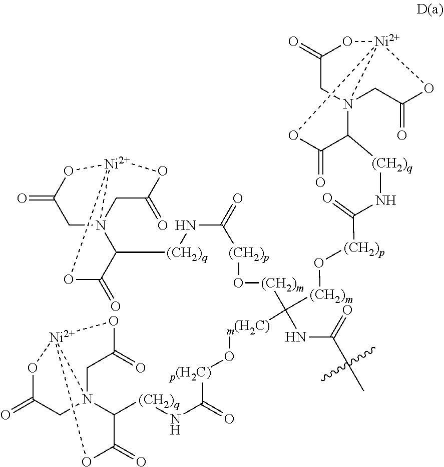

wherein
m, p and q are each independently an integer number between 1 and 8.

In another embodiment, the His-tag binder comprised in the system, the artificial receptor, the recombinant cell, and the methods of the invention comprises a moiety represented by the structure of formula D(b):

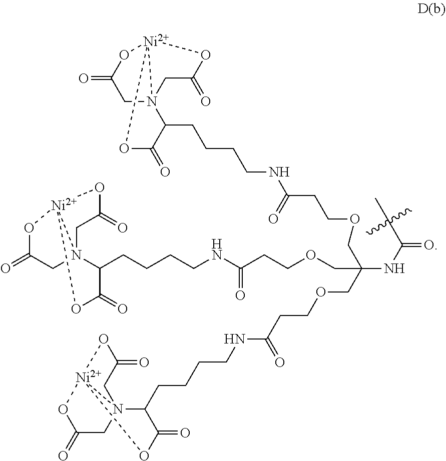

In some embodiments, the His-tag binder comprised in the system, the artificial receptor, the recombinant cell, and the methods of the invention comprises a moiety represented by the structure of formula E:

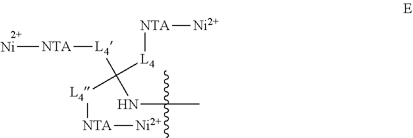

wherein
L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In another embodiment, the His-tag binder comprised in the system, the artificial receptor, the recombinant cell, and the methods of the invention comprises a moiety represented by the structure of formula E(a):

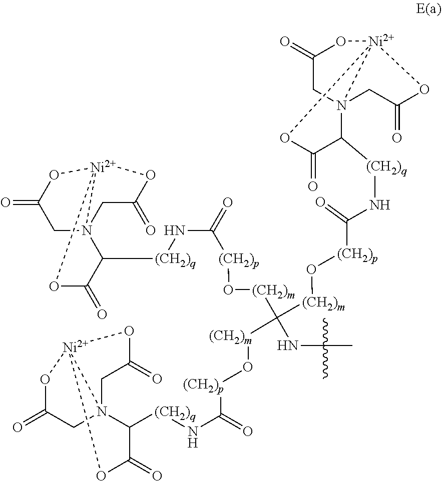

wherein
m, p and q are each independently an integer number between 1 and 8.

In some embodiments, the His-tag binder comprised in the system, the artificial receptor, the recombinant cell, and the methods of the invention comprises a moiety represented by the structure of formula E(b):

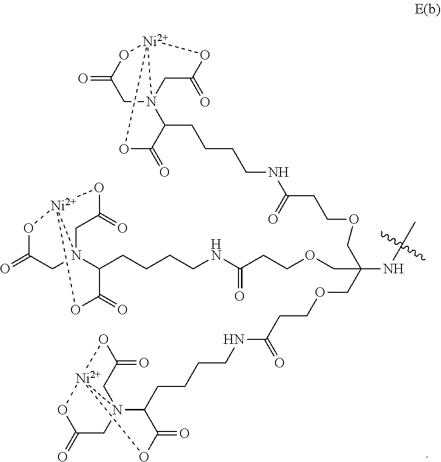

In some embodiments, the His-tag binder comprised in the system, the artificial receptor, the recombinant cell, and the methods of the invention comprises a moiety represented by the structure of formula G:

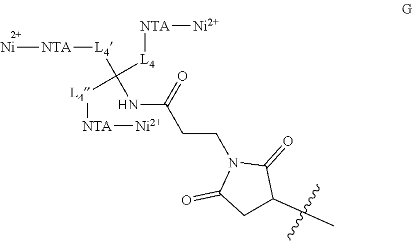

wherein
L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In another embodiment, the His-tag binder comprised in the system, the artificial receptor, the recombinant cell, and the methods of the invention comprises a moiety represented by the structure of formula G(a):

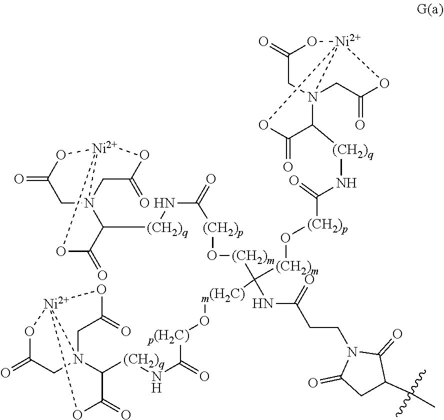

wherein
m, p and q are each independently an integer number between 1 and 8.

In some embodiments, the His-tag binder comprised in the system, the artificial receptor, the recombinant cell, and the methods of the invention comprises a moiety represented by the structure of formula G(b):

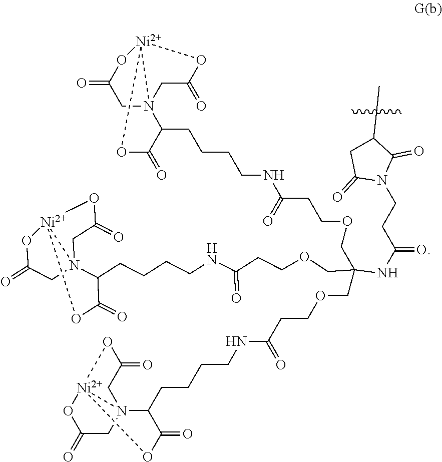

In some embodiments, each of L₄, L₄′, and L₄″ of the structures of formulas D, E, G and/or H, is independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof; each represents a separate embodiment according to this invention. In some embodiments, each of L₄, L₄′, and L₄″ is a combination of alkyl ether and alkyl amide (i.e., alkylether-alkylamide). In another embodiment, each of L₄, L₄′, and L₄″ is independently —(CH₂)_{q}—NHCO—(CH₂)ₚ—O—(CH₂)ₘ—, wherein q, p and m are each independently an integer between 1 and 8. In another embodiment, q is 4, p is 2 and m is 1. In another embodiment, each of L₄, L₄′, and L₄″ is —(CH₂)₄—NHCO—(CH₂)₂O—CH₂—. In another embodiment, each of L₄, L₄′, and L₄″ is represented by the following structure:

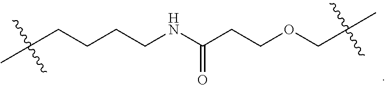 [SMILES: *CCCCNC(=O)CCOC*]

In another embodiment, m of the structures of formulas D(a), E(a), G(a) and/or H(a), is 1. In another embodiment, m is 2. In another embodiment, m is 3. In another embodiment, m is 4.

In another embodiment, p of the structures of formulas D(a), E(a), G(a) and/or H(a) is 1. In another embodiment, p is 2. In another embodiment, p is 3. In another embodiment, p is 4.

In another embodiment, q of the structures of formulas D(a), E(a), G(a) and/or H(a) is 1. In another embodiment, q is 2. In another embodiment, q is 3. In another embodiment, q is 4. In another embodiment, q is 5. In another embodiment, q is 6.

In another embodiment, m is 1, p is 2 and q is 4.

### ODN Sequences

As used herein, “oligonucleotide sequence,” “oligonucleotide” or “nucleic acid sequence” refer to a nucleotide, oligonucleotide, polynucleotide, or any fragment thereof and to naturally occurring or synthetic molecules, such as L-DNA, phosphorothioates, locked nucleic acids, etc.

As used herein, an “oligonucleotide”, “ODN” or “oligonucleotide sequence” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, which do not have a hydroxyl group at the 2′ position, and oligoribonucleotides, which have a hydroxyl group in this position. Oligonucleotides also may include derivatives, in which the hydrogen of the hydroxyl group is replaced with organic groups, e.g., an allyl group. An oligonucleotide is a nucleic acid that includes at least two nucleotides.

One oligonucleotide sequence may be “complementary” to a second oligonucleotide sequence. As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. As an example, for the sequence “T-G-A”, the complementary sequence is “A-C-T.” Complementarity can be “partial,” in which only some of the bases of the nucleic acids are matched according to the base pairing rules. Alternatively, there can be “complete”, “full” or “total” complementarity between the nucleic acids. The degree of complementarity between the oligonucleotide strands has effects on the efficiency and strength of hybridization between the nucleic acid strands.

Oligonucleotides as described herein may be capable of forming hydrogen bonds with oligonucleotides having a complementary base sequence. These bases may include the natural bases such as A, G, C, T and U, as well as artificial bases. An oligonucleotide may include nucleotide substitutions. For example, an artificial or modified base may be used in place of a natural base such that the artificial base exhibits a specific interaction that is similar to the natural base.

An oligonucleotide that is complementary to another nucleic acid will “hybridize” to the nucleic acid under suitable conditions (described below). As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. “Hybridizing” sequences which bind under conditions of low stringency are those which bind under non-stringent conditions (6×SSC/50% formamide at room temperature) and remain bound when washed under conditions of low stringency (2×SSC, 42° C.). Hybridizing under high stringency refers to the above conditions in which washing is performed at 2×SSC, 65° C. (where SSC is 0.15M NaCl, 0.015M sodium citrate, pH 7.2)

In some embodiments, the oligonucleotide sequences of the system, the artificial receptor, the recombinant cell, and the methods according to the invention may each be at least 4, at least 8, at least 12, at least 16, at least 20, or at least 30 nucleotides in length; each is a separate embodiment according to this invention. In illustrative embodiments, oligonucleotide sequences may each be no more than about 50 nucleotides in length. In illustrative embodiments, oligonucleotide sequences may each be no more than about 200 nucleotides in length. In one embodiment, the oligonucleotide sequences, may be partially complementary to a third oligonucleotide, which binds the at oligonucleotide sequences for the formation of larger molecular assemblies.

### ODN-1

In some embodiments, the first oligonucleotide (ODN1) of the system, the artificial receptor, the recombinant cell, and the methods according to the invention is at least 4, at least 8, at least 12, at least 16, at least 20, or at least 30 nucleotides in length; each is a separate embodiment according to this invention. In some embodiments, the first oligonucleotide of the system, the artificial receptor, the recombinant cell, and the methods according to the invention is no more than about 50 nucleotides in length. In some embodiments, the first oligonucleotide is at least 2, at least 4, at least 8, at least 12, at least 16, or at least 20 nucleotides shorter than the second oligonucleotide; each is a separate embodiment according to this invention.

In some embodiments, the first oligonucleotide comprises a sequence comprising at least 80% homology to any of SEQ ID Nos.: 1-5. In some embodiments, the first oligonucleotide sequence is represented by any one of SEQ ID Nos.: 1-5.

### ODN-2

In some embodiments, the second oligonucleotide (ODN2) of the system, the artificial receptor, the recombinant cell, and the methods according to the invention is at least 4, at least 8, at least 12, at least 16, at least 20, or at least 30 nucleotides in length; each is a separate embodiment according to this invention. In some embodiments, the second oligonucleotide of the system, the artificial receptor, the recombinant cell, and the methods according to the invention is no more than about 50 nucleotides in length. In some embodiments, the second oligonucleotide is at least 2, at least 4, at least 8, at least 12, at least 16, or at least 20 nucleotides longer than the first oligonucleotide; each is a separate embodiment according to this invention. In some embodiments, the second oligonucleotide comprises a toehold region.

In some embodiments, the second oligonucleotide comprises a sequence comprising at least 80% homology to any of SEQ ID Nos.: 6-9. In some embodiments, the second oligonucleotide sequence is represented by any one of SEQ ID Nos.: 6-9.

### ODN-3

In some embodiments, the system according to this invention, further comprises a third oligonucleotide (ODN-3).

In some embodiments, ODN-3 is capable of detaching ODN-2 from ODN-1, thereby detaching the second compound according to this invention from the cell of the invention. In some embodiments, the third oligonucleotide is fully complementary to the second oligonucleotide.

In some embodiments, the third oligonucleotide (ODN3) of the system according to the invention is at least 4, at least 8, at least 12, at least 16, at least 20, or at least 30 nucleotides in length. In some embodiments, the third oligonucleotide of the system according to the invention is no more than about 50 nucleotides in length. In some embodiments, the third oligonucleotide is at least 2, at least 4, at least 8, at least 12, at least 16, or at least 20 nucleotides longer than the second oligonucleotide; each is a separate embodiment according to this invention. In some embodiments, the third oligonucleotide has the same length as the second oligonucleotide. In some embodiments, the third oligonucleotide is at least 2, at least 4, at least 8, at least 12, at least 16, or at least 20 nucleotides longer than the first oligonucleotide; each is a separate embodiment according to this invention.

In some embodiments, the third oligonucleotide comprises a sequence comprising at least 80% homology to SEQ ID No.: 10. In some embodiments, the third oligonucleotide sequence is represented by SEQ ID No.: 10.

In some embodiments, ODN-3 is capable of detaching ODN-2 from ODN-1 by a toehold mechanism. In some embodiments, ODN-2 comprises a toehold region complementary to a fragment of ODN-3. A “toehold region” refers to an oligonucleotide segment that comprises a single-stranded overhang that allows detaching two complementary oligonucleotides. In some embodiments, ODN-2 is hybridized to ODN-1, and ODN-2 further comprises a toehold region, which is a single-stranded overhang not complementary of ODN-1. In some embodiments, ODN-2's toehold region is complementary to a fragment of ODN-3. Therefore, in some embodiments, when ODN-3 is added, it binds to ODN-2 toehold region. Once ODN-3 is bound to the toehold region, ODN-3 will compete with ODN-1 for binding the rest of ODN-2's bases. As ODN-1 and ODN-3 exchange base pairs with ODN-2, the branch point of the three-stranded complex moves back and forth. This ‘three-way branch migration’ is an unbiased random walk, as each step causes no net change in base pairing. Eventually, ODN-1 will fully dissociate, and ODN-2 will become fully bound to ODN-3. Thus, in some embodiments, ODN-3 can be used to detach the second compound, ODN-2, or the synthetic agent from the recombinant cell.

### The Second Compound (Y-ODN-2)

In some embodiments, the system, the artificial receptor, the recombinant cell, and the methods of this invention, comprise a second compound comprising a second oligonucleotide (ODN-2) covalently bound to a synthetic agent, either directly or through a second linker, wherein said second oligonucleotide is complementary to said first oligonucleotide.

In some embodiments, the second compound of the system, the artificial receptor, the recombinant cell, and the methods according to this invention (i.e., Y-ODN-2) comprises:
a. a second oligonucleotide (ODN-2), which is complementary to said first oligonucleotide;
b. a synthetic agent,
c. optionally a second linker which links the second oligonucleotide with the synthetic agent;
d. optionally a second labeling moiety;
e. optionally a fourth linker which links the second oligonucleotide with the second labeling moiety.

In some embodiments, the second oligonucleotide is directly bound to the synthetic agent. In other embodiments, the second oligonucleotide is bound to the synthetic agent through a second linker. In some embodiments, the second oligonucleotide is directly bound to the second labeling moiety. In other embodiments, the second oligonucleotide is bound to the second labeling moiety through a fourth linker.

In some embodiments, the second compound according to this invention (i.e., Y-ODN-2) is represented by the structure of formula K:

F₂-L₄-ODN2-L₂-X (K)

wherein
X is a synthetic agent;
L₂ is a second linker or absent;
ODN2 is a second oligonucleotide sequence;
L₄ is a fourth linker or absent; and
F₂ is a second labeling moiety or absent.

In some embodiments, the second compound according to this invention (i.e., Y-ODN-2) is represented by the structure of the following compounds:

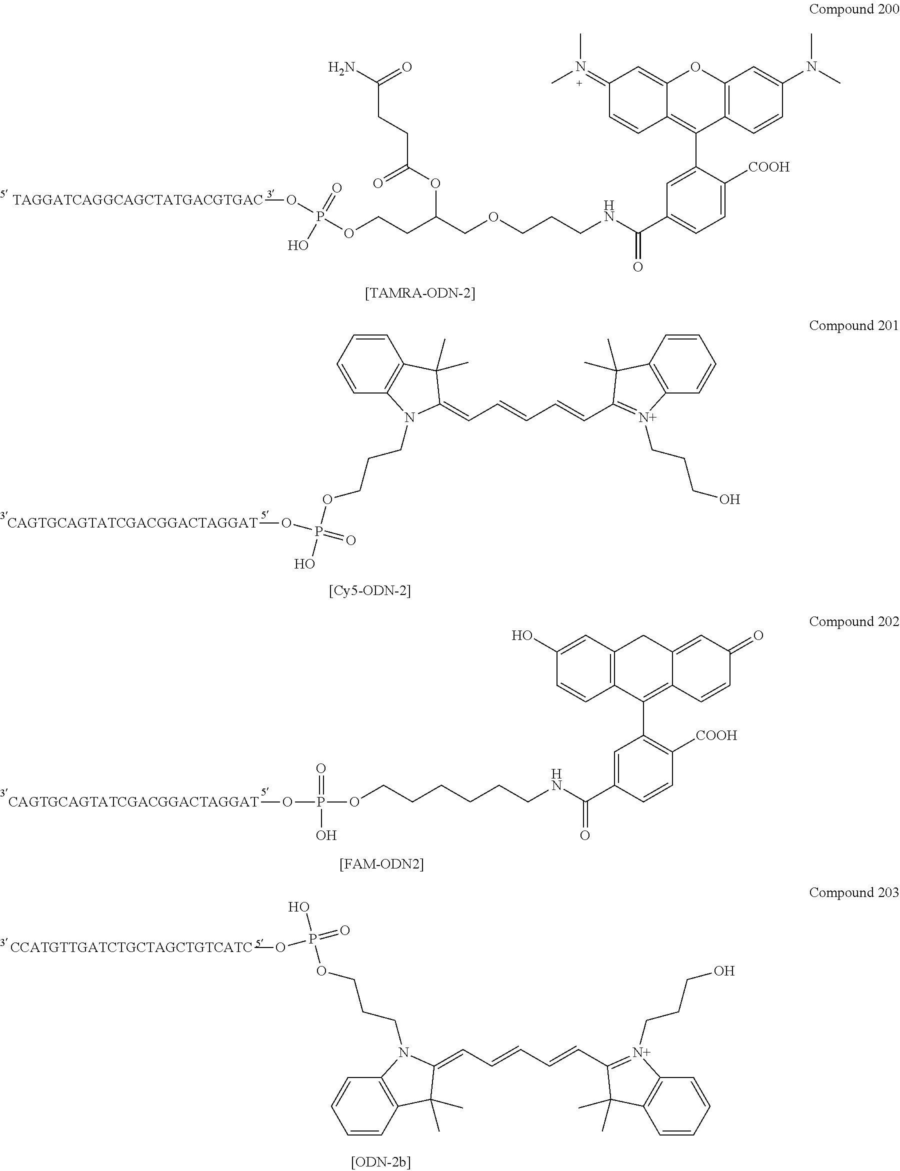

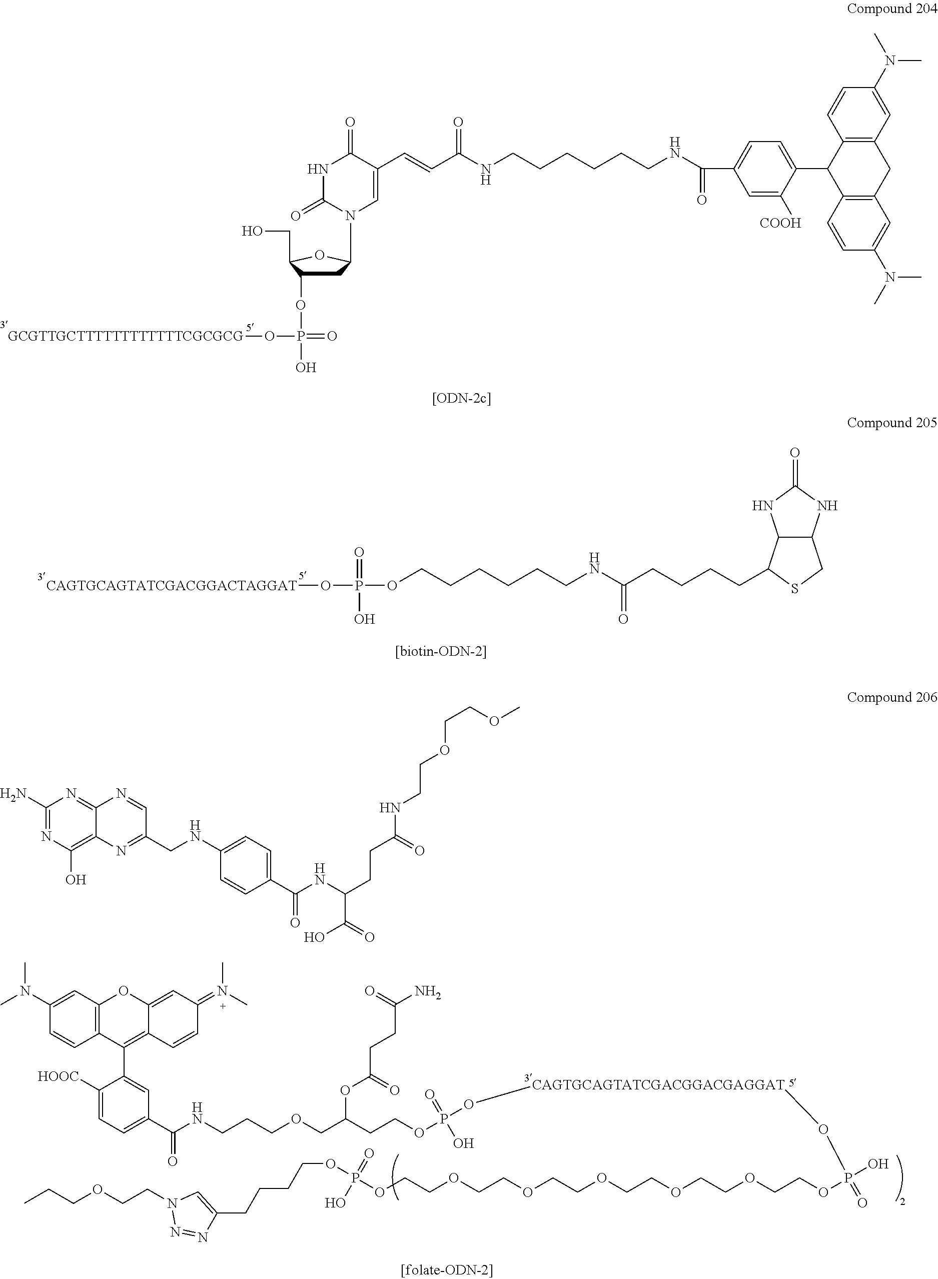

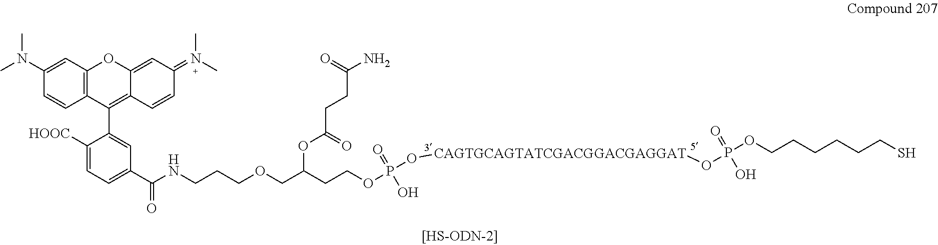

In some embodiments, X of formula K is a synthetic agent. In some embodiments, X is a selective protein binder. In some embodiments, X is a folate. In some embodiments, X is a biotin. In some embodiments, X comprises an adhesion molecule. In some embodiments, X comprises a surface binder. In some embodiments, X comprises an abiotic surface binder. In some embodiments, X comprises an —SH functional group. In some embodiments, X is a thioalkyl. In some embodiments, X is a labeling moiety. In some embodiments, X is a dye. In some embodiments, X is a fluorescent dye. Examples of dyes include but are not limited to: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof. In some embodiments, X is bound to ODN2 through an amide bond, an ester bond, a phosphate bond, an ether bond, a thiolether bond, each represents a separate embodiment according to this invention. In some embodiments, X is covalently bound to ODN2 through a phosphate moiety. In some embodiments, X is bound to L₂ through an amide bond, an ester bond, a phosphate bond, an ether bond, a thioether bond, each represents a separate embodiment according to this invention. In some embodiments, X is as described hereinbelow in the definition of a synthetic agent. In some embodiments, X is a dye derivative. In some embodiments, X is a derivative of a commercially available phosphoramidite dye agent. Non limiting examples of such phosphoramidite dye agents include:

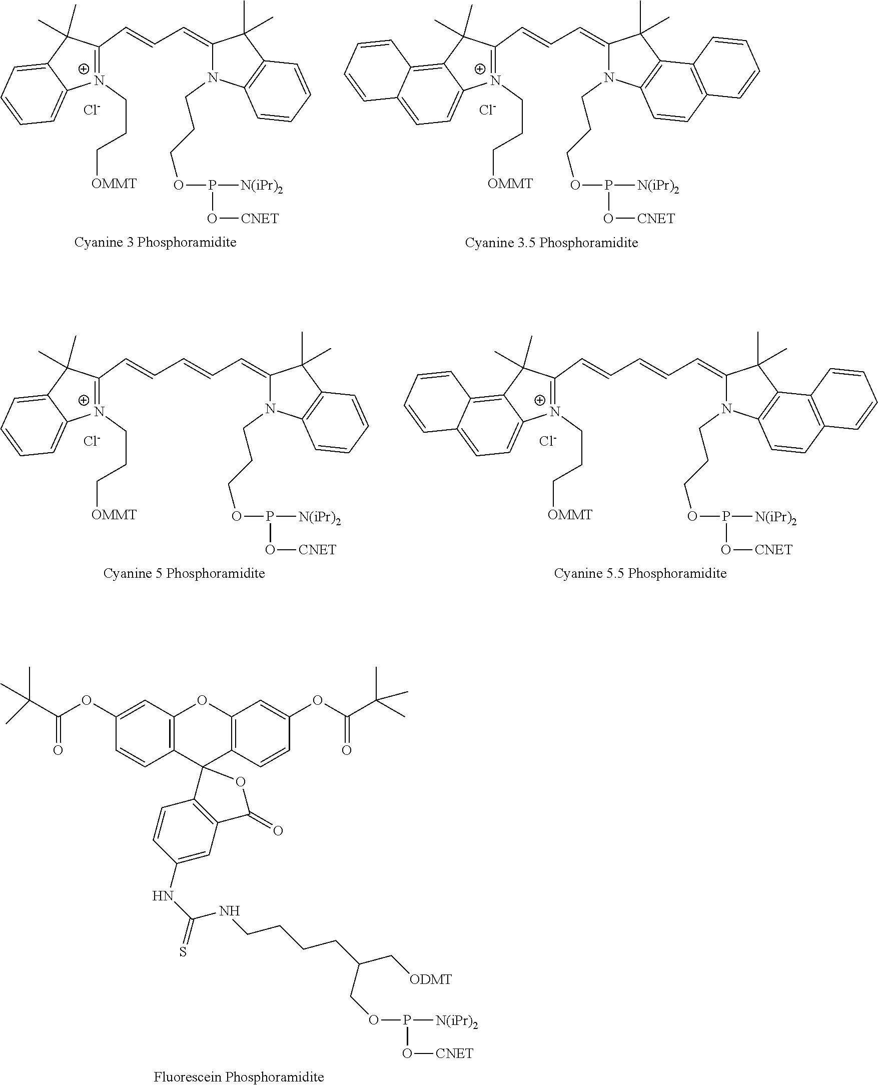

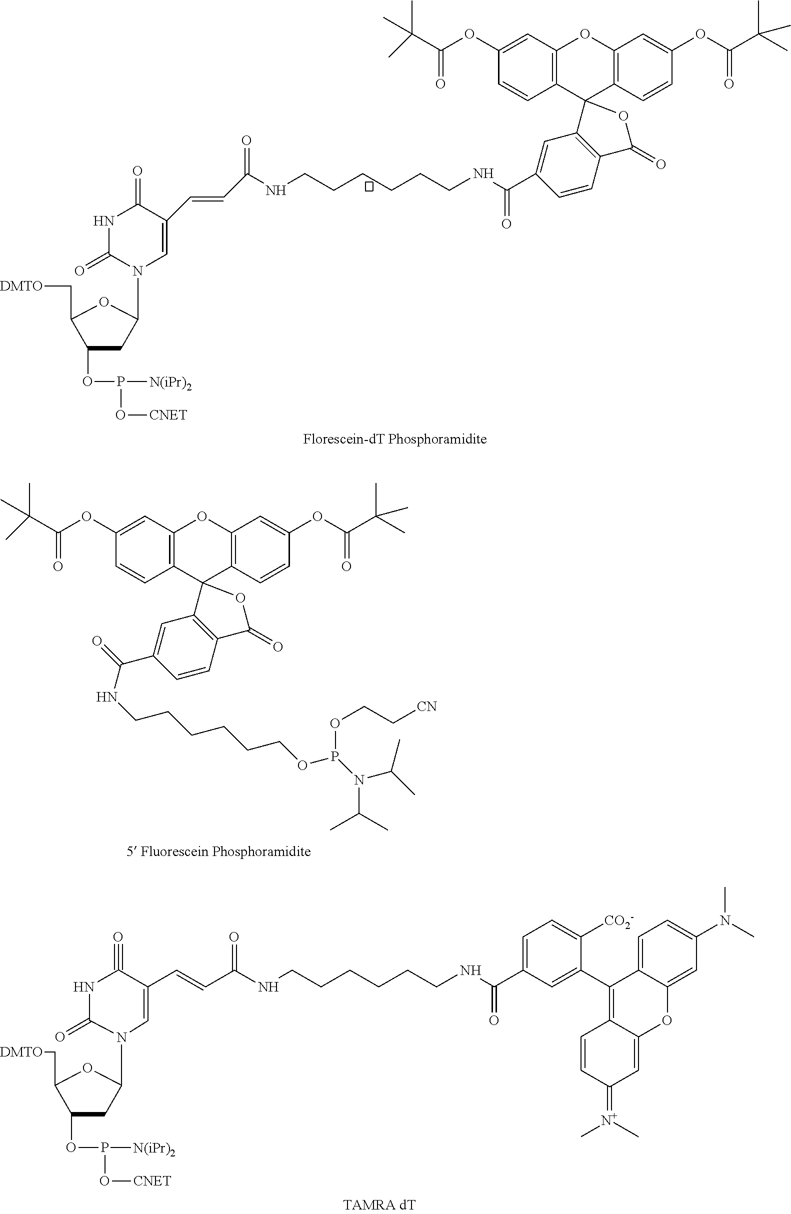

In some embodiments, L₂ of formula K is a second linker. In some embodiments, L₂ is absent. In some embodiments, L₂ is bound to the 3′ end of ODN2. In some embodiments, L₂ is bound to the 5′ end of ODN2. In some embodiments, L₂ is bound to X through an amide bond, an ester bond, a phosphate bond, an ether bond, a thioether bond, each represents a separate embodiment according to this invention. In some embodiments, L₂ is bound to ODN2 through an amide bond, an ester bond, a phosphate bond, an ether bond, a thioether bond, each represents a separate embodiment according to this invention. In some embodiments, L₂ is defined for the “second linker” hereinbelow.

In some embodiments, ODN2 of formulas K is a second oligonucleotide sequence. In some embodiments, ODN2 is directly bound to X, through an amide bond, an ester bond, a phosphate bond, an ether bond, a thioether bond, each represents a separate embodiment according to this invention. In some embodiments, ODN2 is directly bound to F₂, through an amide bond, an ester bond, a phosphate bond, an ether bond, a thioether bond, each represents a separate embodiment according to this invention. In some embodiments, ODN2 is directly bound to F₂, through a phosphate moiety.

In some embodiments, L₄ of formulas K is a fourth linker. In some embodiments, L₄ is absent. In some embodiments, L₄ is bound to the 3′ end of ODN2. In some embodiments, L₄ is bound to the 5′ end of ODN2. In some embodiments, L₄ is bound to F₂ through an amide bond, an ester bond, a phosphate bond, an ether bond, a thioether bond, each represents a separate embodiment according to this invention. In some embodiments, L₄ is bound to ODN2 through an amide bond, an ester bond, a phosphate bond, an ether bond, a thioether bond, each represents a separate embodiment according to this invention. In some embodiments, L₄ is bound to ODN2 through a phosphate moiety. In some embodiments, L₄ is as defined for the “fourth linker” hereinbelow.

In some embodiments, F₂ of formulas K is a second labeling moiety. In some embodiments, F₂ is absent. In some embodiments, F₂ is a dye. Examples of dyes include but are not limited to: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof. In some embodiments, F₂ is a dye derivative. In some embodiments, F₂ is bound to ODN2 through an amide bond, an ester bond, a phosphate bond, an ether bond, a thiolether bond, each represents a separate embodiment according to this invention. In some embodiments, F₂ is bound to L₄ through an amide bond, an ester bond, a phosphate bond, an ether bond, a thioether bond, each represents a separate embodiment according to this invention. In some embodiments, F₂ is as defined for the “labeling moiety” hereinbelow.

Linkers (L₂ and L₄)

In some embodiments, the second compound of the system, the artificial receptor, the recombinant cell, and the methods according to this invention (i.e., Y-ODN-2) comprises:
a. a second oligonucleotide (ODN-2), which is complementary to said first oligonucleotide;
b. a synthetic agent,
c. optionally a second linker which links the second oligonucleotide with the synthetic agent;
d. optionally a second labeling moiety;
e. optionally a fourth linker which links the second oligonucleotide with the second labeling moiety.

### A Second Linker (L₂)

In some embodiments, the second compound (Y-ODN-2) of the system, the artificial receptor, the recombinant cell, and the methods of this invention, comprises a second linker, which links the second oligonucleotide with the synthetic agent. In some embodiments, the second linker is absent. In some embodiments, the second oligonucleotide is directly bound to the synthetic agent. In some embodiments, the second linker is bound to the 3′ end of the second oligonucleotide (ODN2). In some embodiments, the second linker is bound to the 5′ end of ODN2. In some embodiments, the second linker is bound to the synthetic agent through an amide bond, an ester bond, a phosphate bond, an ether bond, a thioether bond; each represents a separate embodiment according to this invention. In some embodiments, the second linker is bound to ODN2 through an amide bond, an ester bond, a phosphate bond, an ether bond, a thioether bond; each represents a separate embodiment according to this invention. In some embodiments, the second linker is covalently bound to the second oligonucleotide through a phosphate moiety.

In some embodiments, the second linker of the system, the artificial receptor, the recombinant cell, and the methods according to this invention and/or L₂ according to formula K is any chemical fragment which comprises at least one segment of a commercially available phosphoramidite spacer derivative as described hereinabove for the “first linker”.

In some embodiments, the second linker of the system, the artificial receptor, the recombinant cell, and the methods according to this invention and/or L₂ according to formula K is a substituted or unsubstituted linear or branched alkyl chain of 1-20 carbon atoms, substituted or unsubstituted linear or branched alkyl ether chain of 1-20 carbon atoms, substituted or unsubstituted linear or branched thioalkyl chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl ester of 1-20 carbon atoms, substituted or unsubstituted linear or branched alkyl diamide chain of 1-20 carbon atoms, substituted or unsubstituted linear or branched alkyl triazole of 1-20 carbon atoms, substituted or unsubstituted linear or branched alkyl amine chain of 1-20 carbon atoms or any combination thereof; each is a separate embodiment according to this invention.

In some embodiments, the second linker of the system, the artificial receptor, the recombinant cell, and the methods according to this invention and/or L₂ according to formula K comprises the following moieties:

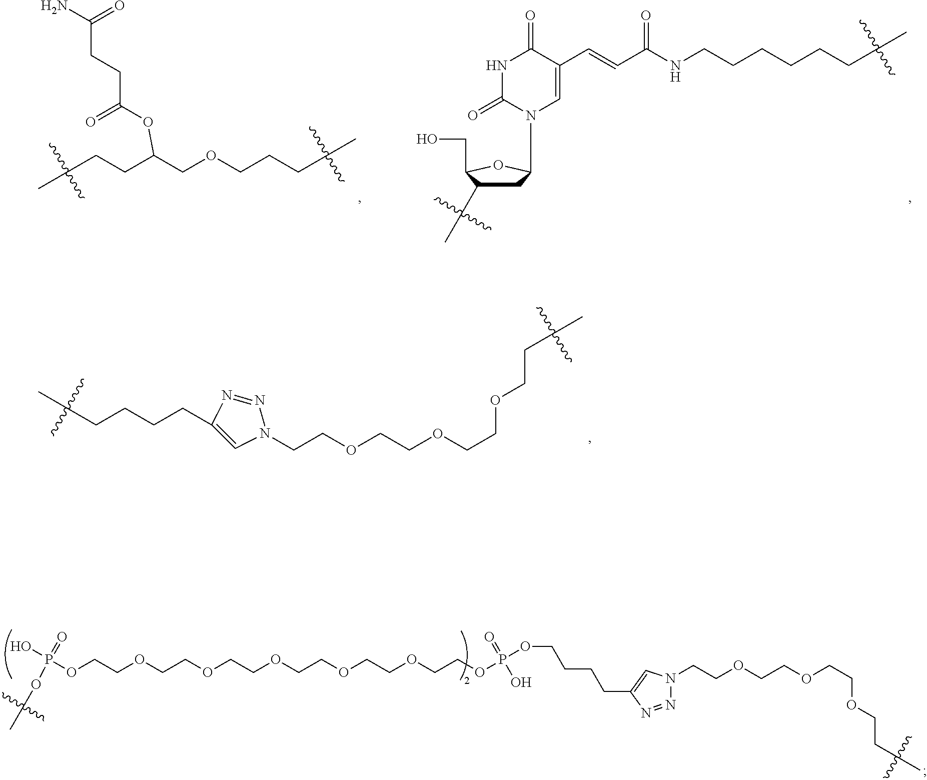

each represent a separate embodiment according to this invention.

In some embodiments, the second linker of the system, the artificial receptor, the recombinant cell, and the methods according to this invention and/or L₂ according to formula K comprises at least one polyethyleneglycol (PEG) moiety, at least one phosphate moiety, at least one thioalkyl moiety; each represents a separate embodiment according to this invention. In some embodiments, the second linker and/or L₂ comprises at least one polyethyleneglycol (PEG) moiety, at least one phosphate moiety, at least one thioalkyl moiety, or any combination thereof.

In some embodiments, the second linker of the system, the artificial receptor, the recombinant cell, and the methods according to this invention and/or L₂ according to formula K is represented by the following formula:

—[(CH₂O)ₖ—PO₃H]—(CH₂)_{w}—S—

wherein
k and l are each independently an integer number between 0 and 10; and
w is an integer number between 1 and 10.

In some embodiments, k is 0. In some embodiments, k is 6. In some embodiments, k is 1, 2, 3, 4, 5, 7, 8, 9, 10; each is a separate embodiment according to this invention.

In some embodiments, 1 is 0. In some embodiments, 1 is 1. In some embodiments, 1 is 5. In some embodiments, 1 is 2, 3, 4, 6, 7, 8, 9, 10; each is a separate embodiment according to this invention.

In some embodiments, w is 6. In some embodiments, w is 1, 2, 3, 4, 5, 7, 8, 9, 10; each is a separate embodiment according to this invention.

### A Fourth Linker (L₄)

In some embodiments, the second compound (Y-ODN-2) of the system, the artificial receptor, the recombinant cell, and the methods according to this invention, comprises a fourth linker, which links the second oligonucleotide with the second labeling moiety. In some embodiments, the second oligonucleotide is directly (covalently) bound to the second labeling moiety. In other embodiments, the second oligonucleotide is covalently bound to the second labeling moiety through a fourth linker. In some embodiments, the fourth linker is absent. In some embodiments, the fourth linker is covalently bound to the 3′ end of ODN-2. In some embodiments, the fourth linker is covalently bound to the 5′ end of ODN-2. In some embodiments, the third linker is a part of a commercially available phosphoramidite dye derivative. In some embodiments, the fourth linker is covalently bound to the second labeling moiety through an amide bond, an ester bond, a phosphate bond, an ether bond, a thioether bond; each represents a separate embodiment according to this invention. In some embodiments, the fourth linker is covalently bound to ODN-2 through an amide bond, an ester bond, a phosphate bond, an ether bond, a thioether bond; each represents a separate embodiment according to this invention. In some embodiments, the fourth linker is covalently bound to the second oligonucleotide through a phosphate moiety.

In some embodiments, the fourth linker of the system, the artificial receptor, the recombinant cell, and the methods according to this invention, and/or L₄ according to formula K is any chemical fragment which comprises at least one segment of a commercially available phosphoramidite spacer derivative as described hereinabove for the “first linker”.

In some embodiments, the fourth linker of the system, the artificial receptor, the recombinant cell, and the methods according to this invention and/or L₄ according to formula K is a substituted or unsubstituted linear or branched alkyl chain of 1-20 carbon atoms, substituted or unsubstituted linear or branched alkyl ether chain of 1-20 carbon atoms, substituted or unsubstituted linear or branched thioalkyl chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl ester of 1-20 carbon atoms, substituted or unsubstituted linear or branched alkyl diamide chain of 1-20 carbon atoms, substituted or unsubstituted linear or branched alkyl triazole of 1-20 carbon atoms, substituted or unsubstituted linear or branched alkyl amine chain of 1-20 carbon atoms or any combination thereof; each is a separate embodiment according to this invention.

In some embodiments, the fourth linker of the system, the artificial receptor, the recombinant cell, and the methods according to this invention and/or L₄ according to formula K comprises the following moieties:

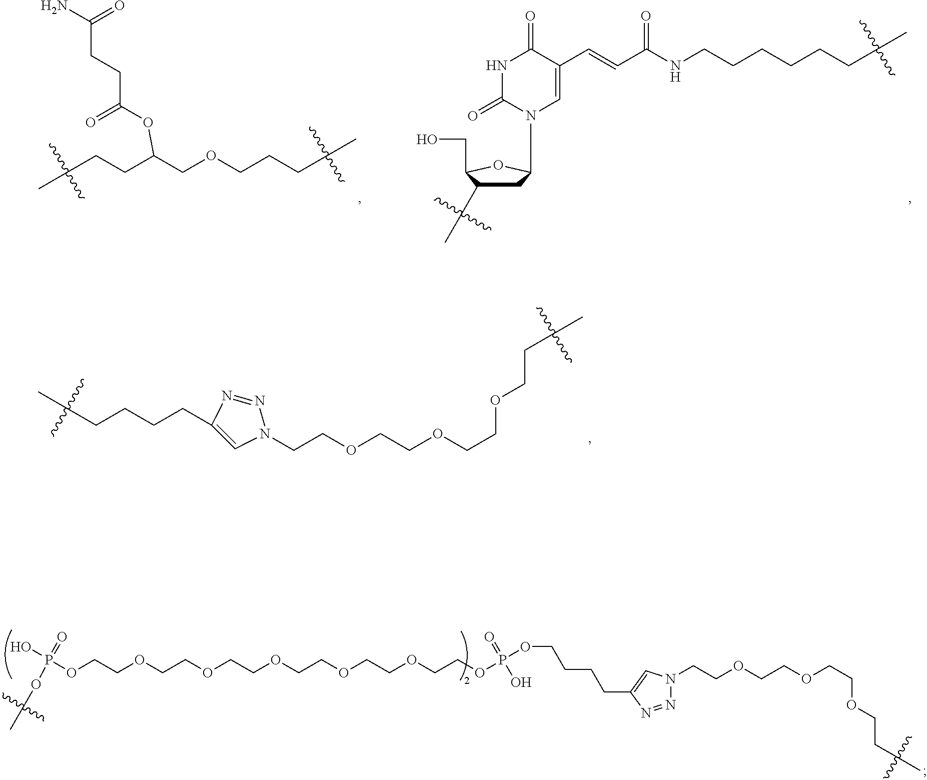

each represent a separate embodiment according to this invention.

In some embodiments, the fourth linker of the system, the artificial receptor, the recombinant cell, and the methods according to this invention and/or L₄ according to formula K comprises at least one polyethyleneglycol (PEG) moiety, at least one phosphate moiety, at least one thioalkyl moiety; each represents a separate embodiment according to this invention. In some embodiments, the fourth linker and/or L₄ comprises at least one polyethyleneglycol (PEG) moiety, at least one phosphate moiety, at least one thioalkyl moiety, or any combination thereof.

In some embodiments, the fourth linker of the system, the artificial receptor, the recombinant cell, and the methods according to this invention and/or L₄ according to formula K is represented by the following formula:

—[(CH₂O)ₖ—PO₃H]—(CH₂)_{w}—S—

wherein
k and l are each independently an integer number between 0 and 10; and
w is an integer number between 1 and 10.

In some embodiments, k is 0. In some embodiments, k is 6. In some embodiments, k is 1, 2, 3, 4, 5, 7, 8, 9, 10; each is a separate embodiment according to this invention.

In some embodiments, 1 is 0. In some embodiments, 1 is 1. In some embodiments, 1 is 5. In some embodiments, 1 is 2, 3, 4, 6, 7, 8, 9, 10; each is a separate embodiment according to this invention.

In some embodiments, w is 6. In some embodiments, w is 1, 2, 3, 4, 5, 7, 8, 9, 10; each is a separate embodiment according to this invention.

As used herein, the term “alkyl” can be any straight- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₈ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof.

The alkyl group can be a sole substituent or it can be a component of a larger substituent, such as in an alkoxy, alkoxyalkyl, haloalkyl, arylalkyl, alkylamino, dialkylamino, alkylamido, alkylurea, thioalkyl, alkyldiamide, alkylamide, alkylphosphate, alkylether, alkyltriazole, alkylester, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, 2, 3, or 4-CH₂—C₆H₄—Cl, C(OH)(CH₃)(Ph), etc.

### Labeling Moiety (F and F₂)

In accordance with the system, the artificial receptor, the recombinant cell, and the methods disclosed herein, the compounds may comprise one or more labeling moieties, which are attached to the oligonucleotides. Oligonucleotides can be labeled by incorporating moieties detectable by one or more means including, but not limited to, spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

As used herein, “labeling moieties” or “labels” are chemical or biochemical moieties useful for labeling a compound. Such labeling moieties include fluorescent agents, chemiluminescent agents, chromogenic agents, quenching agents, radionucleotides, enzymes, substrates, cofactors, inhibitors, nanoparticles, magnetic particles, and other moieties known in the art. Labels are capable of generating a measurable signal and may be covalently or noncovalently joined to an oligonucleotide or nucleotide. In some embodiments, the labeling moieties are covalently bound to the oligonucleotides of the invention. In some embodiments, the labeling moieties are covalently bound to the oligonucleotides of the invention through a linker or a spacer.

In illustrative embodiments, the compounds according to this invention, may be labeled with a “fluorescent dye” or a “fluorophore.” As used herein, a “fluorescent dye” or a “fluorophore” is a chemical group that can be excited by light to emit fluorescence. Some fluorophores may be excited by light to emit phosphorescence. Dyes may include acceptor dyes that are capable of quenching a fluorescent signal from a fluorescent donor dye. In some embodiments, the dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or a derivative thereof. Non limiting examples of Dyes that may be used in the disclosed compounds, system and methods include, but are not limited to, the following dyes and/or dyes sold under the following trade names: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-Hydroxy Tryptamine (HAT); 5-ROX (carboxy-X-rhodamine); 6-Carboxyrhodamine 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue™; Calcium Crimson™; Calcium Green; Calcium Orange; Calcofluor White; Cascade Blue™; Cascade Yellow; Catecholamine; CCF2 (GeneBlazer); CFDA; CFP-Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM Methylcoumarin; CTC; CTC Formazan; Cy2™; Cy3. 1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant Iavin EBG; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARFi; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof; each is a separate embodiment according to this invention.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

In some embodiments, the labeling moiety on the oligonucleotides and the compounds of the system and methods according to the invention, is a quencher. Quenching may include dynamic quenching (e.g., by FRET), static quenching, or both. Illustrative quenchers may include Dabcyl. Illustrative quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

In some situations, it may be useful to include interactive labels on two oligonucleotides with due consideration given for maintaining an appropriate spacing of the labels on the oligonucleotides to permit the separation of the labels during conformational changes. One type of interactive label pair is a quencher-dye pair, which may include a fluorophore and a quencher. The ordinarily skilled artisan can select a suitable quencher moiety that will quench the emission of the particular fluorophore. In an illustrative embodiment, the Dabcyl quencher absorbs the emission of fluorescence from the fluorophore moiety.

Alternatively, the proximity of the two labels can be detected using fluorescence resonance energy transfer (FRET) or fluorescence polarization. FRET is a distance-dependent interaction between the electronic excited states of two dye molecules in which excitation is transferred from a donor molecule to an acceptor molecule without emission of a photon. Examples of donor/acceptor dye pairs for FRET are known in the art and may include fluorophores and quenchers described herein such as Fluorescein/Tetramethylrhodamine, IAEDANS/Fluorescein (Molecular Probes, Eugene, Oreg.), EDANS/Dabcyl, Fluorescein/Fluorescein (Molecular Probes, Eugene, Oreg.), BODIPY FL/BODIPY FL (Molecular Probes, Eugene, Oreg.), BODIPY TMR/ALEXA 647, ALEXA-488/ALEXA-647, and Fluorescein/QSY7™.

The labels can be conjugated to the oligonucleotides directly, or indirectly through linkers or spacers, by a variety of techniques. Depending upon the precise type of label used, the label can be located at the 5′ or 3′ end of the oligonucleotide, located internally in the oligonucleotide's nucleotide sequence, or attached to spacer arms extending from the oligonucleotide and having various sizes and compositions to facilitate signal interactions. According to various embodiments, the labeling moiety is attached to the 5′ or 3′ end of the first and/or the second oligonucleotide; each is a separate embodiment. Using commercially available phosphoramidite reagents, one can produce oligonucleotides containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite.

Oligonucleotides may also incorporate oligonucleotide functionalizing reagents having one or more sulfhydryl, amino or hydroxyl moieties into the oligonucleotide sequence. For example, biotin can be added to the 5′ end by reacting an aminothymidine residue, introduced during synthesis, with an N-hydroxysuccinimide ester of biotin. Labels at the 3′ terminus, for example, can employ polynucleotide terminal transferase to add the desired moiety, such as for example, cordycepin, ³⁵S-dATP, and biotinylated dUTP.

In some embodiments, the first and/or the second compound of the system, the artificial receptor, the recombinant cell, and the methods according to this invention according to this invention, comprises a labeling moiety and/or a second labeling moiety (F of formula H, H(a), H(b) and/or F₂ of formula K, respectively). In some embodiments, the first oligonucleotide (ODN-1) is bound to a labeling moiety in its 3′ or 5′ end. In some embodiments, the labeling moiety is bound to the first oligonucleotide directly. In some embodiments, the labeling moiety is bound to first oligonucleotide through a third linker. In some embodiments, the second oligonucleotide (ODN-2) is bound to a second labeling moiety in its 3′ or 5′ end. In some embodiments, the second labeling moiety is bound to the second oligonucleotide directly. In some embodiments, the second labeling moiety is bound to second oligonucleotide through a fourth linker.

### Synthetic Agent

In some embodiments, the second compound of the system, the artificial receptor, the recombinant cell, and the methods according to this invention, comprises a synthetic agent. In some embodiments, the second oligonucleotide (ODN-2) is bound to a synthetic agent in its 3′ or 5′ end. In some embodiments, the synthetic agent is bound to ODN-2 directly. In some embodiments, the synthetic agent is bound to ODN-2 through a second linker.

In some embodiments, the second compound comprises a synthetic agent and a second labeling moiety. In some embodiments, the second compound does not comprise a second labeling moiety.

According to this invention, the term “synthetic agent” refers to any chemical moiety, which provides a chemical or biological function to the system, or to the cell, to which it is attached. In some embodiments, synthetic agent refers to any chemical moiety, which is capable of binding to various extracellular signals such as ions, small molecules, proteins, and cells, and can control the response of cells to their surroundings. In some embodiments, a synthetic agent refers to any chemical moiety, which has a chemical, physical or biological effect on the cell to which it is attached. In some embodiments, a synthetic agent refers to any chemical moiety, which has a biological effect on a living organism, a tissue or a cell (also referred herein as “a bioactive moiety”). In some embodiments, a biological effect comprises affecting the growth, the survival, the replication, the differentiation, the transcriptome, the proteome, or the function of a cell. In some embodiments, synthetic agent refers to any chemical moiety, which can bind, either covalently or non-covalently, to a solid support, and/or to an abiotic surface (also referred herein as “a surface binder”). In some embodiments, a synthetic agent refers an artificial receptor appended with a specific functionality. In some embodiments, a synthetic agent refers to any chemical moiety, which provides the cell, system or compound to which it is attached, with a specific functionality (e.g., fluorescence, therapeutic effect, solid surface binding capability, specific cell targeting, etc.).

In some embodiments, the synthetic agent is a labeling moiety as described herein above.

In some embodiments, the synthetic agent is a therapeutically active agent. In some embodiments, the therapeutically active agent is a drug. In some embodiments, the therapeutically active agent is selected from: anticancer agents, DNA-interacting molecules, cholesterol-lowering compounds, antibiotics, anti-AIDS molecules, each represents a separate embodiment according to the invention.

In some embodiments, the synthetic agent is a is an oligonucleotide, a nucleic acid construct, an antisense, a plasmid, a polynucleotide, an amino acid, a peptide, a polypeptide, a hormone, a steroid, an antibody, an antigen, a radioisotope, a chemotherapeutic agent, a toxin, an anti-inflammatory agent, a growth factor or any combination thereof; each represents a separate embodiment according to the invention.

In some embodiments, the synthetic agent is a molecular marker. In some embodiments, the synthetic agent is an adhesion molecule. In some embodiments, synthetic agent is a cancer cell binder. In some embodiments, “cancer cell binder” refers to any chemical moiety capable of interacting with proteins expressed by cancer cells. In some embodiments, “cancer cell binder” refers to a protein binder capable of interacting with proteins expressed by cancer cells. In some embodiments, the synthetic agent is a protein ligand. In some embodiments, the synthetic agent is a protein binder. In some embodiments, the synthetic agent is a protein receptor. In some embodiments, the synthetic agent is a drug. In some embodiments, the synthetic agent is an anticancer agent. In some embodiments, the synthetic agent is a growth factor. In some embodiments, the synthetic agent is a surface binder. In some embodiments, the synthetic agent is an abiotic surface binder. In some embodiments, the surface binder is a functional group capable of binding a solid surface or a solid support.

In some embodiments, the synthetic agent is a protein binder. In some embodiments, a “protein binder” refers to any biological research reagent which binds to a specific target protein. Non limiting examples of protein binders known in the art include: drugs, folate, biotin, marimastat, ethacrynic acid, bisethacrynic acid, Ni-nitrilotriacetic acid (Ni-NTA), bis Ni-NTA, tris-Ni-NTA, PDGF-BB, heparin, FGF aptamer, estrogen, DNA aptamer, RNA aptamer, peptide aldehyde, estrogen, suberoylanilidehydroxamic acid (SAHA), or a peptide binder; each represents a separate embodiment according to this invention.

In some embodiments, the synthetic agent is a cancer cell binder. In some embodiments, the cancer cell binder is a folate.

In some embodiments, the synthetic agent is a molecular marker. In some embodiments, the synthetic agent is an angiogenic factor. In some embodiments, the synthetic agent is a cytokine. In some embodiments, the synthetic agent is a hormone. In some embodiments, the synthetic agent is a DNA molecule. In some embodiments, the synthetic agent is a siRNA molecule. In some embodiments, the synthetic agent is an oligosaccharide.

In some embodiments, the synthetic agent is a protein receptor. In some embodiments, the synthetic agent is an immune activator. In some embodiments, the synthetic agent is an immune suppressor. In some embodiments, the synthetic agent is a small molecule. In some embodiments, the small molecule is a drug.

In some embodiments, the synthetic agent is a labeling moiety as described herein above. In some embodiments, the labeling moiety is a dye. In some embodiments, the dye is a fluorescent dye. In some embodiments, the dye is selected from a group consisting of: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or a derivative thereof.

In some embodiments, the synthetic agent is a protein receptor. In some embodiments, the synthetic agent is a protein binder. In some embodiments, the synthetic agent is a biotin.

In some embodiments, the synthetic agent is a surface binder. In some embodiments, the synthetic agent is an abiotic surface binder. In some embodiments, the synthetic agent is a binder for abiotic surfaces. In some embodiments, the synthetic agent is an agent capable of binding to solid support. In some embodiments, the surface binder is capable of binding a surface. According to this invention, a “surface binder” is any chemical moiety, or functional group, that is capable of binding solid surfaces. In some embodiments, the binding is covalent, electrostatic, van der Waals or any combination thereof; each is a separate embodiment. In some embodiments, attachment of the surface binder to the surface comprises covalent bond, coordination bond, polar bond, van der Waals bond or any combination thereof. In some embodiments, the surface binder comprises a functional moiety capable of binding a surface. According to this aspect and in some embodiments, the surface binder comprises a thiol end group (SH) or an end group comprising a sulfur-sulfur bond (—S—S—). Such bonds are capable of binding to a noble metal. For example, thiol or —S—S— moieties binds strongly to gold surfaces and to other noble metal surface including but not limited to silver, platinum and palladium. Thiols and —S—S— bonds also bind to semiconductor surfaces such as GaAs etc. In some embodiments, the surface binder comprises a thiol group (HS). In some embodiments, the surface binder is a C₁-C₂₀ thioalkyl. In some embodiments, the surface binder is a C₂-C₈ thioalkyl. In some embodiments, the surface binder is a thiohexyl. In some embodiments, attachment of the surface binder to a surface comprise silicon chemistry. According to this aspect and in some embodiments, the surface is or comprises silicon. In some embodiments, the surface comprises silicon oxide. In some embodiments, the silicon oxide surface comprises glass or quartz. In some embodiments, the surface comprises silicon coated by a silicon oxide layer. According to this aspect and in some embodiments, the surface binder comprises a functional group capable of binding to silicon oxide. In some embodiments, the functional group comprises silicon atom. In some embodiments, the functional group comprises silicon bonded to a halogen atom. In some embodiments, the halogen atom is Cl, Br, F or I. In one embodiment the silicon-halogen functional group comprise Si-trichloride, Si-tribromide, Si-dichloride, Si dibromide. In some embodiments, the functional group comprises Si bonded to oxygen atom. In some embodiments, the functional group comprises Si bonded to two or three oxygen atoms. In some embodiments, the functional group of the surface binder comprises Si-halogen bond and upon reaction with the surface, the halogen atom is replaced by an oxygen atom, and bonding to the surface occurs. In some embodiments, the surface binder comprises a pyridine moiety.

In some embodiments, the surface is an abiotic surface. In some embodiments, the surface is a passivated. In some embodiments, surfaces of this invention are inorganic (e.g. silicon oxide, gold). In some embodiments, surfaces of this invention are organic (e.g. an organic polymer). In some embodiments, surfaces of this invention are metals (e.g., gold). In some embodiments, surfaces of this invention comprise both organic and inorganic materials. In some embodiments, the surface is a material selected from gold, glass, a doped glass, indium tin oxide (ITO)-coated glass, silicon, a doped silicon, Si(100), Si(111), SiO₂, SiH, silicon carbide mirror, quartz, a metal, metal oxide, a mixture of metal and metal oxide, group IV elements, mica, a polymer such as polyacrylamide and polystyrene, a plastic, a zeolite, a clay, wood, a membrane, an optical fiber, a ceramic, a metalized ceramic, an alumina, an electrically-conductive material, a semiconductor, steel or a stainless steel; each is a separate embodiment according to the invention. In some embodiments, the surface is a gold surface. In some embodiments, the surface is a passivated gold surface. In some embodiments, surfaces of this invention are flat. In some embodiments, the surfaces are curved. In some embodiments, the surface is macroscopically flat and microscopically curved or vice-versa. In some embodiments, the surface is the surface of a particle. In some embodiments, the surface is the surface of a nanoparticle.

### Methods for Decorating a Cell

In some embodiments, this invention is directed to a method for decorating a cell with a synthetic agent, said method comprises:

a. ectopically expressing in said cell a polypeptide, wherein said polypeptide comprises a membranal anchoring domain and an extracellular binding domain,
b. incubating the cell of (a) with a first compound comprising a first oligonucleotide (ODN-1) covalently bound to a binder, either directly or through a first linker, said binder comprising affinity to said extracellular binding domain, and
c. incubating the cell of (b) with a second compound comprising a second oligonucleotide (ODN-2) covalently bound to a synthetic agent, either directly or through a second linker, wherein said second oligonucleotide is complementary to said first oligonucleotide; thereby decorating said cell with said synthetic agent.

In some embodiments, this invention is directed to a method for modifying a cell with a synthetic agent, said method comprises:
a. ectopically expressing in said cell a polypeptide, wherein said polypeptide comprises a membranal anchoring domain and an extracellular binding domain,
b. incubating the cell of (a) with a first compound comprising a first oligonucleotide (ODN-1) covalently bound to a binder, either directly or through a first linker, said binder comprising affinity to said extracellular binding domain, and
c. incubating the cell of (b) with a second compound comprising a second oligonucleotide (ODN-2) covalently bound to a synthetic agent, either directly or through a second linker, wherein said second oligonucleotide is complementary to said first oligonucleotide;
   thereby modifying said cell with said synthetic agent.

A skilled artisan would appreciate that “decorating” a cell with a compound or a molecule comprises attaching a number of such molecules to the cell surface. In some embodiments the cell surface is a cell membrane. In some embodiments, the terms “decorating”, “modifying”, “attaching”, “incorporating”, and “binding” are used herein interchangeably, having all the same meanings.

In some embodiments, the methods disclosed herein are applicable to any type of cells. In some embodiments, the cell is an eukaryote cell, a prokaryote cell, a mammalian cell, a plant cell, a human cell, and a bacteria cell. In some embodiments, the cell is E. coli.

In some embodiments, cells are transformed with a construct encoding a polypeptide comprising a membranal anchoring domain and an extracellular binding domain. In some embodiments, said anchoring domain comprises OmpC, and said binding domain comprises a His-tag as described herein above. In some embodiments, transformed cells are cultured to saturation in a growth medium, such as LB supplemented with antibiotics at 30° C. In some embodiments, cells are incubated until the OD₆₀₀ reaches about 0.6, then the expression of the polypeptide is induced by addition of an inducer, such as Rhamnose or isopropyl-b-D-1-thiogalactopyranoside (IPTG), letting cultures to grow further.

Recombinant cells expressing the polypeptide are then collected, in some embodiments, by centrifugation at 6,000 g for 4 min, washed, and resuspended in the same buffer to an OD₆₀₀ of 0.3. A preincubated sample of a first molecule comprising a first oligonucleotide (ODN-1) can be added to a sample of the cell suspension. In some embodiments, 500 nM of ODN-1 and 2.5 μM of NiCl₂ can be added to the cells, which can then be incubated in some embodiments for 1 hour.

After a first compound is bound to the cell membrane, cells can be incubated with a second compound comprising a second oligonucleotide (ODN-2), wherein ODN-2 is complementary to ODN-1. Cells ODN-2 can be added in some embodiments at a concentration of 500 nM and incubated in some embodiments for 30 min.

In some embodiments, a second oligonucleotide ODN-2 can be detached from ODN-1 and from the recombinant cells by adding a third compound comprising a third oligonucleotide ODN-3, wherein ODN-3 is complementary to ODN-2. In some embodiments, ODN-3 can be added at a concentration of 2 μM and incubated for 2 h.

In some embodiments, a first, a second, or a third compound comprising a first, a second, or a third oligonucleotide, respectively, is added at a concentration lower than about 5 nM, between about 5 nM and 50 nM, between about 50 nM and 500 nM, between about 500 nM and 5 μM, between about 5 μM and 50 μM, between about 50 μM and 500 μM, or higher than 500 μM.

In some embodiments, the first compound comprising the first oligonucleotide (ODN-1) can in some embodiments be removed from the cell surface by incubating the cells with EDTA. In some embodiments, incubating the cells with about 5 mM or about 10 mM EDTA for 1 hour detaches the first compound from the cell surface. Cells can then be collected by centrifugation and washed.

In some embodiments, the cell is a living cell. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(a), D(b), E, E(a), E(b), G, G(a), or G(b). In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof; each represents a separate embodiment according to this invention. In some embodiments, the second compound further comprises a second labeling moiety. In some embodiments, the second labeling moiety comprises a fluorescent dye.

In some embodiments, the method is for decorating a cell surface. In some embodiments, the method is for decorating a cell membrane. In some embodiments, the method is for modifying a cell surface. In some embodiments, the method is for modifying a cell membrane. In some embodiments, the synthetic agent is a labeling moiety. In some embodiments, the synthetic agent is a fluorescent dye. In some embodiments, the synthetic agent is a surface binder. In some embodiments, the synthetic agent is an abiotic surface binder. In some embodiments, the synthetic agent is a thioalkyl. In some embodiments, the synthetic agent is a protein binder. In some embodiments, the synthetic agent is a biotin. In some embodiments, the synthetic agent is a cancer cell binder. In some embodiments, the synthetic agent is a folate. In some embodiments, the binder is a His-tag binder. In some embodiments, the His-tag binder is represented by the structure of formula C, D, D(a), D(b), E, E(a), E(b), G, G(a), G(b).

### Methods for Adhering a First Cell to a Second Cell

In some embodiments, this invention is directed to a method for adhering a first cell to a second cell, said method comprises incubating a recombinant cell according to this invention, with a second cell, wherein the synthetic agent is an adhesion molecule.

In some embodiments, this invention is directed to a method for binding a first cell to a second cell, said method comprises incubating a recombinant cell according to this invention, with a second cell, wherein the synthetic agent is an adhesion molecule. In another embodiment, the synthetic agent is a protein binder.

In some embodiments, this invention is directed to a method for adhering a first cell to a second cell, said method comprises incubating a cell ectopically expressing a polypeptide according to this invention, wherein said polypeptide comprises a membranal anchoring domain and an extracellular binding domain, with a first compound according to this invention and with a second compound according to this invention, wherein the synthetic agent is an adhesion molecule, thereby forming a complex according to this invention, following by incubating the formed complex with a second cell, thereby adhering a first cell to a second cell.

In some embodiments, this invention is directed to a method for binding a first cell to a second cell, said method comprises incubating a cell ectopically expressing a polypeptide according to this invention, wherein said polypeptide comprises a membranal anchoring domain and an extracellular binding domain, with a first compound according to this invention and with a second compound according to this invention, wherein the synthetic agent is a protein binder, thereby forming a complex according to this invention, following by incubating the formed complex with a second cell, thereby binding a first cell to a second cell.

In some embodiments, this invention is directed to a method for adhering a first cell to a second cell, said method comprises:

a. ectopically expressing in the first cell a polypeptide, wherein said polypeptide comprises a membranal anchoring domain and an extracellular binding domain,
b. incubating the cell of (a) with a first compound according to this invention comprising a first oligonucleotide (ODN-1) covalently bound to a binder, either directly or through a first linker, said binder comprises affinity to said extracellular binding domain, and
c. incubating the cell of (b) with a second compound according to this invention comprising a second oligonucleotide (ODN-2) covalently bound to an adhesion molecule, either directly or through a second linker, wherein said second oligonucleotide is complementary to said first oligonucleotide, and said adhesion molecule comprises affinity to a compound present on the surface of said second cell,
d. incubating said first cell with said second cell, thereby adhering said first cell to said second cell.

In some embodiments, this invention is directed to a method for binding a first cell to a second cell, said method comprises:
a. ectopically expressing in the first cell a polypeptide, wherein said polypeptide comprises a membranal anchoring domain and an extracellular binding domain,
b. incubating the cell of (a) with a first compound according to this invention comprising a first oligonucleotide (ODN-1) covalently bound to a binder, either directly or through a first linker, said binder comprises affinity to said extracellular binding domain, and
c. incubating the cell of (b) with a second compound according to this invention comprising a second oligonucleotide (ODN-2) covalently bound to an adhesion molecule, either directly or through a second linker, wherein said second oligonucleotide is complementary to said first oligonucleotide, and said adhesion molecule comprises affinity to a compound present on the surface of said second cell,
d. incubating said first cell with said second cell, thereby binding said first cell to said second cell.

In some embodiments, the adhesion molecule is a protein binder.

In some embodiments, the recombinant cell is selected from a group comprising eukaryotes, prokaryotes, mammalian cells, plant cells, human cells, and bacteria. In some embodiments, a mammalian or a human cell is selected from a group comprising epithelial cells, Brunner's gland cells in duodenum, insulated goblet cells of respiratory and digestive tracts, stomach, foveolar cells, chief cells, parietal cells, pancreatic acinar cells, Paneth cells of small intestine, Type II pneumocyte of lung, club cells of lung, barrier cells, type i pneumocytes, gall bladder epithelial cells, centroacinar cells, intercalated duct cells, intestinal brush border cells, hormone-secreting cells, enteroendocrine cells, K cells, L cells, I cells, G cells, enterochromaffin cells, enterochromaffin-like cells, N cells, S cells, D cells, Mo cells, thyroid gland cells, thyroid epithelial cells, parafollicular cells, parathyroid gland cells, parathyroid chief cells, oxyphil cells, pancreatic islets, alpha cells, beta cells, delta cells, epsilon cells, PP cells, salivary gland mucous cells, salivary gland serous cells, Von Ebner's gland cells in tongue, mammary gland cells, lacrimal gland cells, ceruminous gland cells in ear, eccrine sweat gland dark cells, eccrine sweat gland clear cells, apocrine sweat gland cells, gland of moll cells in eyelid, sebaceous gland cells, Bowman's gland cells in nose, hormone-secreting cells, anterior/intermediate pituitary cells, corticotropes, gonadotropes, lactotropes, melanotropes, somatotropes, thyrotropes, magnocellsular neurosecretory cells, parvocellsular neurosecretory cells, chromaffin cells, keratinocytes, epidermal basal cells, melanocytes, trichocytes, medullary hair shaft cells, cortical hair shaft cells, cuticular hair shaft cells, huxley's layer hair root sheath cells, Henle's layer hair root sheath cells, outer root sheath hair cells, surface epithelial cells of cornea, tongue, mouth, nasal cavity, distal anal canal, distal urethra, and distal vagina, basal cells, intercalated duct cells, striated duct cells, lactiferous duct cells, ameloblast, auditory inner hair cells of organ of Corti, auditory outer hair cells of organ of Corti, basal cells of olfactory epithelium, primary sensory neurons, Merkel cells of epidermis, olfactory receptor neuron, pain-sensitive primary sensory neurons, photoreceptor cells of retina in eye, proprioceptive primary sensory neurons, touch-sensitive primary sensory neurons, chemoreceptor glomus cells of carotid body cells, outer hair cells of vestibular system of ear, inner hair cells of vestibular system of ear, taste receptor cells of taste bud, neuron cells, interneurons, basket cells, cartwheel cells, Stellate cells, Golgi cells, granule cells, Lugaro cells, unipolar brush cells, Martinotti cells, chandelier cells, Cajal-Retzius cells, double-bouquet cells, neurogliaform cells, retina horizontal cells, amacrine cells, spinal interneuron, renshaw cells, spindle neurons, fork neurons, pyramidal cells, place cells, grid cells, speed cells, head direction cells, Betz cells, stellate cells, boundary cells, bushy cells, Purkinje cells, medium spiny neurons, astrocytes, oligodendrocytes, ependymal cells, tanycytes, pituicytes, adipocytes, white fat cells, brown fat cells, liver lipocytes, cells of the adrenal cortex, cells of the zona glomerulosa, cells of the zona fasciculata, cells of the zona reticularis, theca interna cells of ovarian follicle, granulosa lutein cells, theca lutein cells, leydig cells of testes, seminal vesicle cells, prostate gland cells, bulbourethral gland cells, Bartholin's gland cells, gland of littre cells, uterus endometrium cells, juxtaglomerular cells, macula densa cells of kidney, peripolar cells of kidney, mesangial cells of kidney, parietal epithelial cells, podocytes, proximal tubule brush border cells, loop of Henle thin segment cells, kidney distal tubule cells, kidney collecting duct cells, principal cells, intercalated cells, transitional epithelium, duct cells, efferent ducts cells, epididymal principal cells, epididymal basal cells, endothelial cells, planum semilunatum epithelial cells of vestibular system of ear, organ of Corti interdental epithelial cells, loose connective tissue fibroblasts, corneal fibroblasts, tendon fibroblasts, bone marrow reticular tissue fibroblasts, other nonepithelial fibroblasts, pericytes, hepatic stellate cells, nucleus pulposus cells of intervertebral disc, hyaline cartilage chondrocytes, fibrocartilage chondrocytes, elastic cartilage chondrocytes, osteoblast/osteocytes, osteoprogenitor cells, hyalocyte of vitreous body of eye, stellate cells of perilymphatic space of ear, pancreatic stellate cells, red skeletal muscle cells, white skeletal muscle cells, intermediate skeletal muscle cells, nuclear bag cells of muscle spindle, nuclear chain cells of muscle spindle, myosatellite cells, cardiac muscle cells, cardiac muscle cells, node cells, Purkinje fiber cells, smooth muscle cells, myoepithelial cells of iris, myoepithelial cells of exocrine glands, erythrocytes, megakaryocytes, platelets, monocytes, connective tissue macrophage, epidermal Langerhans cells, osteoclast, dendritic cells, microglial cells, neutrophil granulocytes, eosinophil granulocytes, basophil granulocytes, hybridoma cells, mast cells, helper T cells, suppressor T cells, cytotoxic T cells, natural killer T cells, B cells, natural killer cells, reticulocytes, hematopoietic stem cells and committed progenitors for the blood and immune system, oogonium/oocytes, spermatids, spermatocytes, spermatogonium cells, spermatozoon, and interstitial kidney cells.

In some embodiments, the second cell comprises a cellular pathology. In some embodiments, the second cell is a cancer cell. In some embodiments, the cancer is selected from: a carcinoma, a sarcoma, a lymphoma, leukemia, a germ cell tumor, a blastoma, chondrosarcoma, Ewing's sarcoma, malignant fibrous histiocytoma of bone/osteosarcoma, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoetic cancer, lymphoma, Non-hodgkin lymphoma, skin cancer, basal-cell carcinoma, melanoma, small cell lung cancer, non-small cell lung cancer, or any combination thereof.

In some embodiments, the adhesion molecule is any compound that comprises affinity to a compound present in the membrane of a second cell. In some embodiments, the adhesion molecule is selected according to its binding potency to a molecule known to be expressed in a second cell. In some embodiments, the adhesion molecule is any adhesion molecule known in the art.

In some embodiments, the adhesion molecule is a peptide, a polypeptide, a protein or a part thereof. In some embodiments, the adhesion molecule comprises an integrin or a fragment thereof. In some embodiments, the adhesion molecule comprises an immunoglobulin (Ig) or a fragment thereof. In some embodiments, the adhesion molecule comprises a cadherin, or a fragment thereof. In some embodiments, the adhesion molecule comprises a selectins, or a fragment thereof. In some embodiments, the adhesion molecule comprises a calcium-dependent cell adhesion molecule, or a fragment thereof. In some embodiments, the adhesion molecule comprises a proteoglycan, or a fragment thereof. A skilled artisan would appreciate that adhesion molecule recognizes a different ligand.

In some embodiments an adhesion molecule is selected from a group comprising VLA1, VLA2, VLA3, VLA4, VLA5, VLA6, FLJ25220, RLC, HsT18964, FLJ39841, HUMINAE, LFA1A, MAC-1, VNRA, MSK8, GPIIb, FNRB, MSK12, MDF2, LFA-1, MAC-1, MFI7, GP3A, GPIIIa, FLJ26658, fibronectin receptor, laminin receptor, LFA-1, CR3, fibrinogen receptor; gpIIbIIIa, vitronectin receptor, CDH1, CDH2, CDH12, CDH3, DSG1, DSG2, DSG3, DSG4, Desmocollin, DSC1, DSC2, DSC3, Protocadherins, IgSF CAMs, NCAMs, ICAM-1, CD2, CD58, CD48, CD150, CD229, CD244, E-selectin, L-selectin, P-selectin, any fragment thereof, or any combination thereof.

In some embodiments, the cell adhesion molecule comprises a folate. In some embodiments, the second cell expresses an extracellular folate receptor on its surface.

In some embodiments, the first cell is a living cell. In some embodiments, the second cell is a living cell. In some embodiments, the second cell is a cancer cell. In some embodiments, the second cell expresses an extracellular protein receptor on its surface. In some embodiments, the adhesion molecule is a protein binder. In some embodiments, the adhesion molecule is a folate. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(a), D(b), E, E(a), E(b), G, G(a), or G(b). In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the second compound further comprises a second labeling moiety. In some embodiments, the second labeling moiety comprises a fluorescent dye.

### Methods for Adhering a Cell to a Surface

In some embodiments, this invention is directed to a method for adhering a cell to a surface, said method comprises incubating a recombinant cell according to the invention, with a first compound according to the invention, following by incubating the formed cell with a second compound according to this invention, wherein the synthetic agent is a surface binder.

In some embodiments, this invention is directed to a method for adhering a cell to a surface, said method comprises:
a. ectopically expressing in a cell a polypeptide, wherein said polypeptide comprises a membranal anchoring domain and an extracellular binding domain,
b. incubating the cell of (a) with a first compound according to this invention comprising a first oligonucleotide (ODN-1) covalently bound to a binder, either directly or through a first linker, said binder comprises affinity to said extracellular binding domain,
c. incubating the cell of (b) with a second compound according to this invention comprising a second oligonucleotide (ODN-2) covalently bound to a surface binder, either directly or through a second linker, wherein said second oligonucleotide is complementary to said first oligonucleotide, and said surface binder is capable of binding to said surface, and
d. applying said cell to said surface under conditions sufficient for the binding of said surface binder to said surface,
   thereby adhering said cell to said surface.

In some embodiments, the cell is a living cell. In some embodiments, the cell is a bacteria. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(a), D(b), E, E(a), E(b), G, G(a), or G(b). In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the second compound further comprises a second labeling moiety. In some embodiments, the second labeling moiety comprises a fluorescent dye.

In some embodiments, the surface binder is an abiotic surface binder. In some embodiments, the surface is a solid support. In some embodiments, the surface is a passivated. In some embodiments, the surface is a material selected from gold, glass, a doped glass, indium tin oxide (ITO)-coated glass, silicon, a doped silicon, Si(100), Si(111), SiO₂, SiH, silicon carbide mirror, quartz, a metal, metal oxide, a mixture of metal and metal oxide, group IV elements, mica, a polymer such as polyacrylamide and polystyrene, a plastic, a zeolite, a clay, wood, a membrane, an optical fiber, a ceramic, a metalized ceramic, an alumina, an electrically-conductive material, a semiconductor, steel or a stainless steel; each is a separate embodiment according to the invention. In some embodiments, the surface is a gold surface. In some embodiments, the surface binder is a C₁-C₂₀ thioalkyl. In some embodiments, the surface binder is a C₂-C₈ thioalkyl. In some embodiments, the surface binder is a thiohexyl. In some embodiments, the surface binder is a pyridine-terminated moiety.

### Methods for Inducing Luminescent in a Cell

In some embodiments, this invention is directed to a method for inducing luminescence in a cell, said method comprises incubating a recombinant cell according to the invention, with a first compound according to the invention, following by incubating the formed cell with a second compound according to this invention, wherein the synthetic agent is a luminescent moiety.

In some embodiments, this invention is directed to a method for inducing luminescence in a cell, said method comprises:

a. ectopically expressing in a cell a first polypeptide, wherein said polypeptide comprises a membranal anchoring domain and an extracellular binding domain,
b. incubating the cell of (a) with a first compound according to this invention, comprising a first oligonucleotide (ODN-1) covalently bound to a binder, either directly or through a first linker, said binder comprises affinity to said extracellular binding domain, and
c. incubating the cell of (b) with a second compound according to this invention, comprising a second oligonucleotide (ODN-2) covalently bound to a luminescent molecule, either directly or through a second linker, wherein the second oligonucleotide is complementary to the first oligonucleotide,
thereby inducing luminescence in said cell.

Any luminescent molecule can be used in the methods disclosed herein. In some embodiments, the luminescent molecule is as described for a “labeling moiety” herein above. In some embodiments, the luminescent molecule is a fluorescent dye. Examples of fluorescent dyes are given herein above. In some embodiments, the dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC and derivatives thereof.

In some embodiments, the cell is a living cell. In some embodiments, the cell is a bacteria. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(a), D(b), E, E(a), E(b), G, G(a), or G(b). In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the second compound further comprises a second labeling moiety. In some embodiments, the second labeling moiety comprises a fluorescent dye.

### Methods for Binding a Cell to a Protein

In some embodiments, this invention is directed to a method for binding a cell to a protein of interest (POI), said method comprises incubating a recombinant cell according to this invention, with said POI, wherein the synthetic agent is a protein binder.

In some embodiments, this invention is directed to a method for binding a cell to a protein of interest (POI), said method comprises incubating a cell ectopically expressing a polypeptide according to this invention, wherein said polypeptide comprises a membranal anchoring domain and an extracellular binding domain, with a first compound according to this invention and with a second compound according to this invention, thereby forming a complex according to this invention, following by incubating the formed complex with a POI, wherein the synthetic agent is a protein binder, thereby binding a cell to a protein of interest (POI).

In some embodiments, this invention is directed to a method for binding a cell to a protein of interest (POI), said method comprises:
a. ectopically expressing in a cell a polypeptide, wherein said polypeptide comprises a membranal anchoring domain and an extracellular binding domain,
b. incubating the cell of (a) with a first compound according to this invention, comprising a first oligonucleotide (ODN-1) covalently bound to a binder, either directly or through a first linker, said binder comprises affinity to said extracellular binding domain, and
c. incubating the cell of (b) with a second compound according to this invention, comprising a second oligonucleotide (ODN-2) covalently bound to a protein binder, either directly or through a second linker, wherein said second oligonucleotide is complementary to said first oligonucleotide, and said protein binder is selective to said POI, and
d. incubating said cell with said POI,
   thereby binding said cell to said POI.

In some embodiments, the cell is a living cell. In some embodiments, the cell is a bacteria. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(a), D(b), E, E(a), E(b), G, G(a), or G(b). In some embodiments, the first compound is represented by the structure of formula J, H, H(a) and H(b) and compounds 100-104. In some embodiments, the second compound is represented by the structure of formula K and compounds 200-207. In some embodiments, the first linker comprises at least one polyethyleneglycol (PEG) moiety, at least one phosphate moiety, at least one thioalkyl moiety or any combination thereof. In some embodiments, the first compound further comprises a labeling moiety. In some embodiments, the labeling moiety is a fluorescent dye. In some embodiments, the second compound further comprises a second labeling moiety. In some embodiments, the second labeling moiety comprises a fluorescent dye.

In some embodiments, the protein binder is a small molecule ligand. In some embodiments, the protein binder is a peptide, polypeptide a protein, or a part thereof; each is a separate embodiment. In some embodiments, the protein binder is a biotin. In some embodiments, the protein binder is a folate.

### Methods for Treating a Disease

In some embodiments, the recombinant cells disclosed herein comprise a therapeutic effect and are delivered to a patient in need thereof. When used therapeutically, the recombinant cells are referred to herein as “therapeutics”. Methods of administration of therapeutics include, but are not limited to, intravenal, intradermal, intraperitoneal, or surgical routes. The therapeutics of the disclosure presented herein may be administered by any convenient route, for example by infusion, by bolus injection, by surgical implantation and may be administered together with other biologically-active agents. Administration can be systemic or local. It may also be desirable to administer the therapeutic locally to the area in need of treatment; this may be achieved by, for example, and not by way of limitation, local infusion during surgery, by injection, by means of a catheter, or by means of an implant.

A skilled artisan would appreciate that a therapeutically effective amount of the cells may encompass total the amount of cells that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the a therapeutically effective amount refers to that ingredient alone. When applied to a combination, the term refers to combined amounts of the active ingredients that result in the therapeutic effect, whether administered in combination, serially or simultaneously.

In some embodiments, suitable dosage ranges of the therapeutics of the disclosure presented herein are generally between 1 million and 2 million recombinant cells. In some embodiments, suitable doses are between 2 million and 5 million recombinant cells. In some embodiments, suitable doses are between 5 million and 10 million recombinant cells. In some embodiments, suitable doses are between 10 million and 25 million recombinant cells. In some embodiments, suitable doses are between 25 million and 50 million recombinant cells. In some embodiments, suitable doses are between 50 million and 100 million recombinant cells. In some embodiments, suitable doses are between 100 million and 200 million recombinant cells. In some embodiments, suitable doses are between 200 million and 300 million recombinant cells. In some embodiments, suitable doses are between 300 million and 400 million recombinant cells. In some embodiments, suitable doses are between 400 million and 500 million recombinant cells. In some embodiments, suitable doses are between 500 million and 600 million recombinant cells. In some embodiments, suitable doses are between 600 million and 700 million recombinant cells. In some embodiments, suitable doses are between 700 million and 800 million recombinant cells. In some embodiments, suitable doses are between 800 million and 900 million recombinant cells. In some embodiments, suitable doses are between 900 million and 1 billion recombinant cells. In some embodiments, suitable doses are between 1 billion and 2 billion recombinant cells. In some embodiments, suitable doses are between 2 billion and 3 billion recombinant cells. In some embodiments, suitable doses are between 3 billion and 4 billion recombinant cells. In some embodiments, suitable doses are between 4 billion and 5 billion recombinant cells.

One skilled in the art would appreciate that effective doses may be extrapolated from dose-response curves derived from in vitro or animal model test systems.

In some embodiments, recombinant cells are decorated in vitro before delivering to a patient. In some embodiments, recombinant cells are decorated in vivo. In some embodiments, cells are decorated in vivo by first delivering to a patient the recombinant cells, then delivering a first compound that binds the extracellular binding domain of the cells, and then delivering a second compound that binds the first compound. In some embodiments, recombinant cells can proliferate after being delivered to a patient.

The herein-described recombinant cells, either decorated or not, can be incorporated into pharmaceutical compositions suitable for administration. Such compositions typically comprise a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition disclosed here is formulated to be compatible with its intended route of administration. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, polyalcohols such as mannitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, the recombinant cells are prepared with carriers that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers.

In some embodiments, this invention is directed to a kit comprising:

a. a recombinant cell ectopically expressing a polypeptide according to this invention, wherein said polypeptide comprises a membranal anchoring domain and an extracellular binding domain, said extracellular binding domain bound to
b. a first compound according to this invention, comprising a first oligonucleotide (ODN-1) covalently bound to a binder according to this invention, either directly or through a first linker, said binder comprises affinity to said extracellular binding domain,
c. a second compound according to this invention, comprising a second oligonucleotide (ODN-2) covalently bound to a synthetic agent, either directly or through a second linker, wherein said second oligonucleotide is complementary to said first oligonucleotide.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

The following examples are presented in order to more fully illustrate the preferred embodiments of the invention. They should in no way be construed, however, as limiting the broad scope of the invention.

### EXAMPLES

### Example 1—Materials and Methods

All reagents and solvents were obtained from commercial suppliers. Oligonucleotides were obtained from W. M. Keck Foundation Biotechnology at Yale University, which were synthesized using standard automated solid-phase synthesis. Aluminum-backed silica plates (Merck silica gel 60 F254) were used for thin layer chromatography (TLC) to monitor solution-phase reactions. The ¹H-NMR spectra were recorded using a 300 MHz Bruker Avance NMR spectrometer. Chemical shifts are reported in ppm on the 6 scale down field from TMS as the internal standard. The following abbreviations were used to describe the peaks: s-singlet, d-doublet, t-triplet, q-quartet, quin-quintet, and m-multiplet. Electronspray mass spectrometry was performed with a Micromass Platform LCZ-4000 instrument at the Weizmann Institute of Science mass spectrometry facility. Matrix-assisted laser desorption ionization time-of-flight (MALDI-TOF) mass spectrometry was performed on an AB SCIEX 5800 system, equipped with an Nd: YAG (355 nm) laser with a 1 KHz pulse (Applied Biosystems), at the Weizmann Institute of Science mass spectrometry facility. The purification of oligonucleotides was carried out on a Waters 2695 separation module HPLC system with a 2994 photodiode array detector using either a Waters XBridge™ OST C18 column (2.5 μM, 4.6 mm×50 mm) or an XBridge™ OST C18 column (2.5 μM, 10 mm×50 mm). Oligonucleotide samples were desalted using illustra MicroSpin G-25 Columns (GE Healthcare) according to the supplier's instructions. Concentrations of the oligonucleotides were quantified based on their respective electronic absorption at 260 nm and the molar extinction coefficient of the oligonucleotide at this wavelength. Cell images were acquired using an Olympus IX51 fluorescent microscope equipped with a U-MNIBA3 fluorescence filter cube (excitation and emission filters of 470-495 nm, and 510-550 nm, respectively), a U-MNG2 fluorescence filter cube narrow-band (excitation and emission filters of 530-550 nm, and 590 nm, respectively) and a U-MF2 fluorescence filter cube (excitation and emission filters of 620-660 nm, and 700-775 nm, respectively).

### Synthetic Procedures

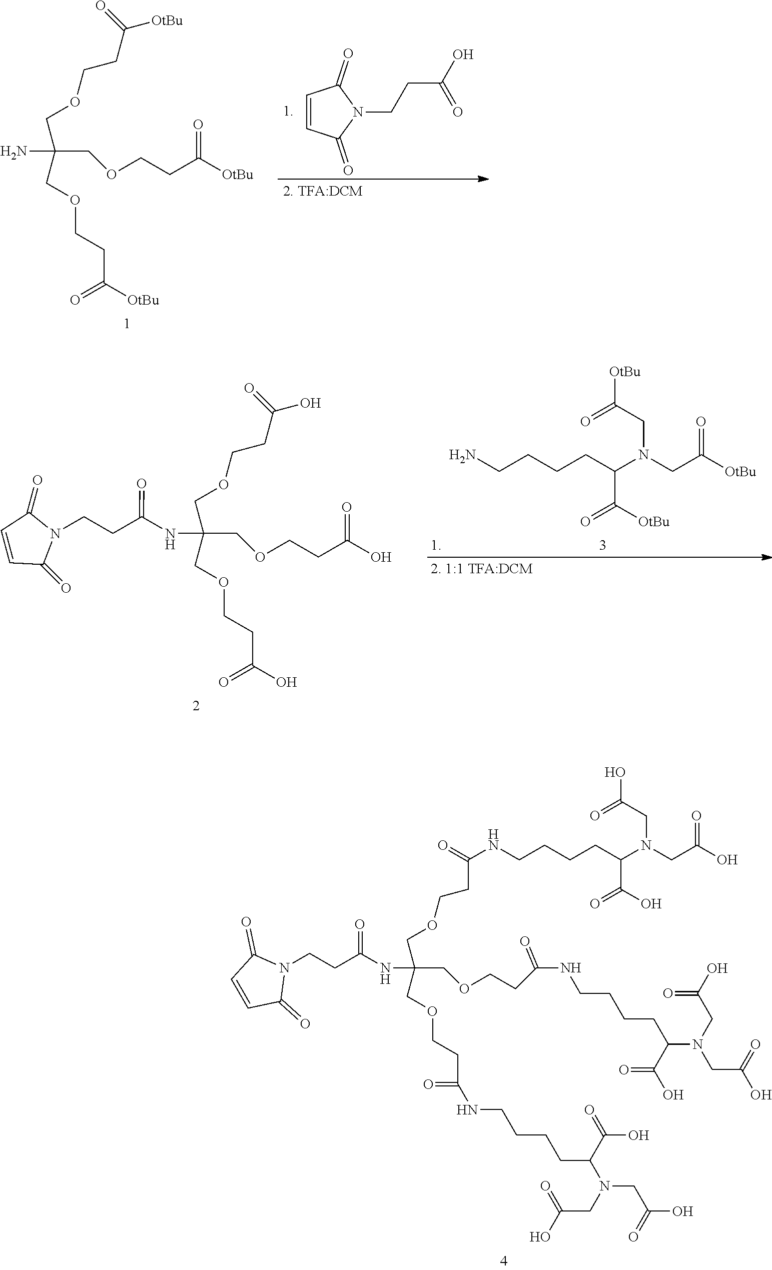 [SMILES: CC(C)(C)OC(=O)CCOCC(N)(COCCC(=O)OC(C)(C)C)COCCC(=O)OC(C)(C)C ; O=C(O)CCN1C(=O)C=CC1=O ; O=C(O)CCOCC(COCCC(=O)O)(COCCC(=O)O)NC(=O)CCN1C(=O)C=CC1=O ; CC(C)(C)OC(=O)CN(CC(=O)OC(C)(C)C)C(CCCCN)C(=O)OC(C)(C)C ; O=C(O)CN(CC(=O)O)C(CCCCNC(=O)CCOCC(COCCC(=O)NCCCCC(C(=O)O)N(CC(=O)O)CC(=O)O)(COCCC(=O)NCCCCC(C(=O)O)N(CC(=O)O)CC(=O)O)NC(=O)CCN1C(=O)C=CC1=O)C(=O)O]

Compounds 1 and 3 were synthesized according to previously reported procedures (Cardona, C. M. An improved synthesis of a trifurcated newkome-type monomer and orthogonally protected two-generation dendrons. J. Org. Chem. 67, 1411-1413 (2002); Huang, Z. Facile synthesis of multivalent nitrilotriacetic acid (nta) and nta conjugates for analytical and drug delivery applications. Bioconjugate Chem. 17, 1592-1600 (2006).

Compound 2: Compound 1 (600 mg, 1.18 mmol) was dissolved in dry DCM (30 ml) under argon and cooled to 0° C. Then, EDC (339 mg, 1.7 mmol) and DIPEA (413.7 μl, 2.32 mmol) were added and the reaction mixture was stirred for 30 min at room temperature. 3-Maleimidopropionic acid (240.1 mg, 1.4 mmol) was added, and the solution was stirred overnight. Then 40 ml DCM was added, and the solution was washed with water (10 ml), and brine (10 ml). The organic layer was dried with Na₂SO₄, filtered, and concentrated under high vacuum. Finally, the crude product was purified by column chromatography (DCM/MeOH, 97:3) to yield a yellow oil (501.6 mg, 64%). ¹H NMR (CDCl₃, 300 MHz): δ 1.44 (s, 27H); 2.44 (t, J=6 Hz, 6H); 2.51 (t, J=6 Hz, 2H); 3.63 (t, J=6 Hz, 6H); 3.67 (s, 6H); 3.80 (t, J=6 Hz, 6H); 6.69 (s, 2H). ESI-MS (m/z): calcd. for (M+H): 657.35, found 657.44; calcd. for (M+Na): 679.35, found 679.31. The tert-butyl groups were then deprotected using a 1:1 (v/v) mixture of TFA: DCM for 2.5 h. After removing the solvents, the excess of TFA was co-evaporated 4 times with DCM and then the product was dried under high vacuum. ¹H NMR (D₂O, 300 MHz): δ 2.47 (t, J=6 Hz, 2H); 2.59 (t, J=6 Hz, 6H); 3.61 (s, 6H); 3.67-3.75 (m, 8H); 6.83 (s, 2H). ESI-MS (m/z): calcd. for (M+H): 489.16, found 489.18; calcd. for (M+Na): 511.16, found 511.12; calcd. for (2M+H): 977.32, found 977.03; calcd. for (2M+Na): 999.32, found 999.15 (2M+Na).

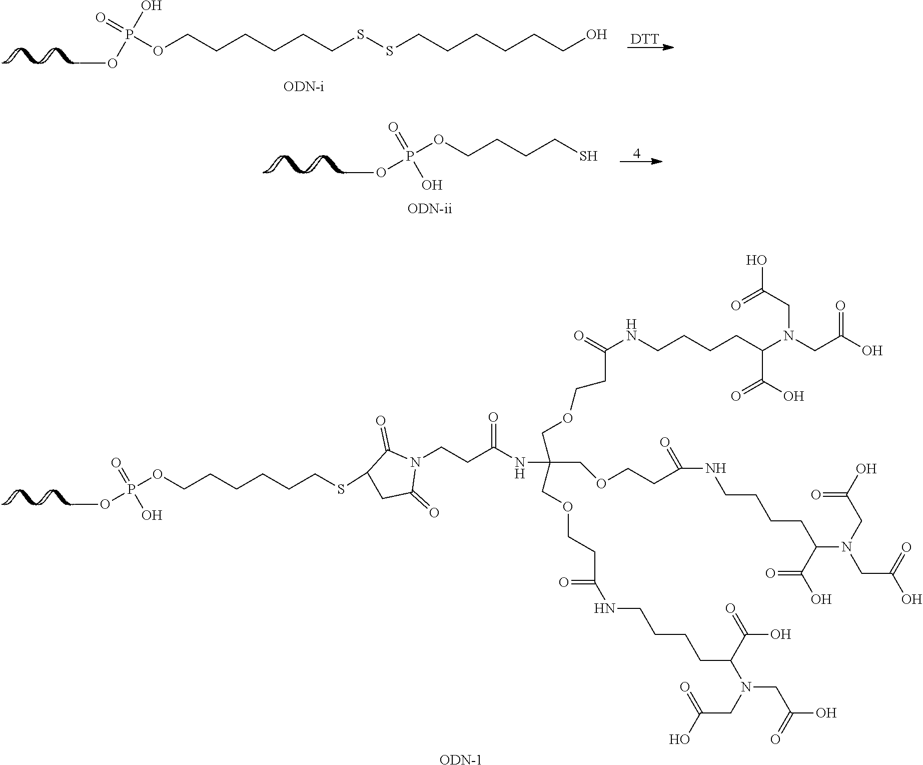

Compound 4: A solution of compound 2 (160 mg, 304.8 μmol) in dry DCM (10 ml) was cooled to 0° C. in an ice bath and DIPEA (212 μl, 1.2 mmol), EDC (191 mg, 1 mmol), and HOBt (41 mg, 304.8 μmol) were added consecutively. After 15 min, compound 3 (433 mg, 1 mmol) was added and the reaction was stirred overnight. Then DCM (40 ml) was added and the solution was washed with water (10 ml). The organic layer was dried with Na₂SO₄, filtered, and concentrated at high vacuum. Finally, the crude product was purified by column chromatography (DCM/MeOH, 96:4) to yield a colorless oil (96.6 mg, 18.3%). ¹H NMR (MeOD, 300 MHz): δ 1.50 (s, 54H); 1.55 (s, 27H); 1.71 (m, 18H); 2.42 (t, J=6 Hz, 6H); 2.49 (m, 2H); 3.20 (t, J=6 Hz, 6H); 3.31 (m, 12H); 3.55-3.74 (m, 17H); 6.84 (s, 2H). ESI-MS (m/z): calcd. for (M+Na): 1749.13, found 1748.72; calcd. for (M+2Na): 886.06, found 886.27; calcd. for (M+3Na): 598.37, found 598.52. The tert-butyl groups were then deprotected using a 1:1 (v/v) mixture of TFA: DCM for 2.5 h. After removing the solvents, the excess of TFA was co-evaporated 4 times with DCM and then the product was dried under high vacuum ¹H NMR (MeOD, 300 MHz): δ 1.47 (m, 6H); 1.53 (m, 6H); 1.91 (m, 6H); 2.43 (m, 8H); 3.17 (m, 6H); 3.58-3.65 (m, 15H); 4.1 (m, 14H); 6.82 (s, 2H). ESI-MS (m/z): calcd. for (M+H): 1221.48, found 1221.53; calcd. for (M+Na): 1243.48, found 1243.39. HRMS.

### General Procedure for the Synthesis of the ODN-1 Strands:

ODN-i (200 nmol) was treated with 400 μl of a DTT solution (50 mM DTT in 50 mM Tris buffer, pH 8.3) for 1 hour. The reduced oligonucleotide (ODN-ii) was then desalted on Sephadex™ G-25 and dried under reduced pressure. ODN-ii was added to a solution of 4 (8 mg) in concentrated PBS×10, pH 7. The reaction was stirred overnight. The product was purified using RP-HPLC. MALDI-TOF MS (m/z): X-ODN-1: calcd. 6319.6, found 6334.2; ODN-1: calcd. 8876.1, found 8893.3; Compound 101: calcd. 11453.6, found 11454.3; Compound 103: calcd. 9139.8, found 9139.2; Compound 104: calcd. 9119.9, found 9115.9.

Compounds 100-104 where synthesized according to the general synthesis described hereinabove.

### Synthesis of Folate-ODN-2 (Compound 206):

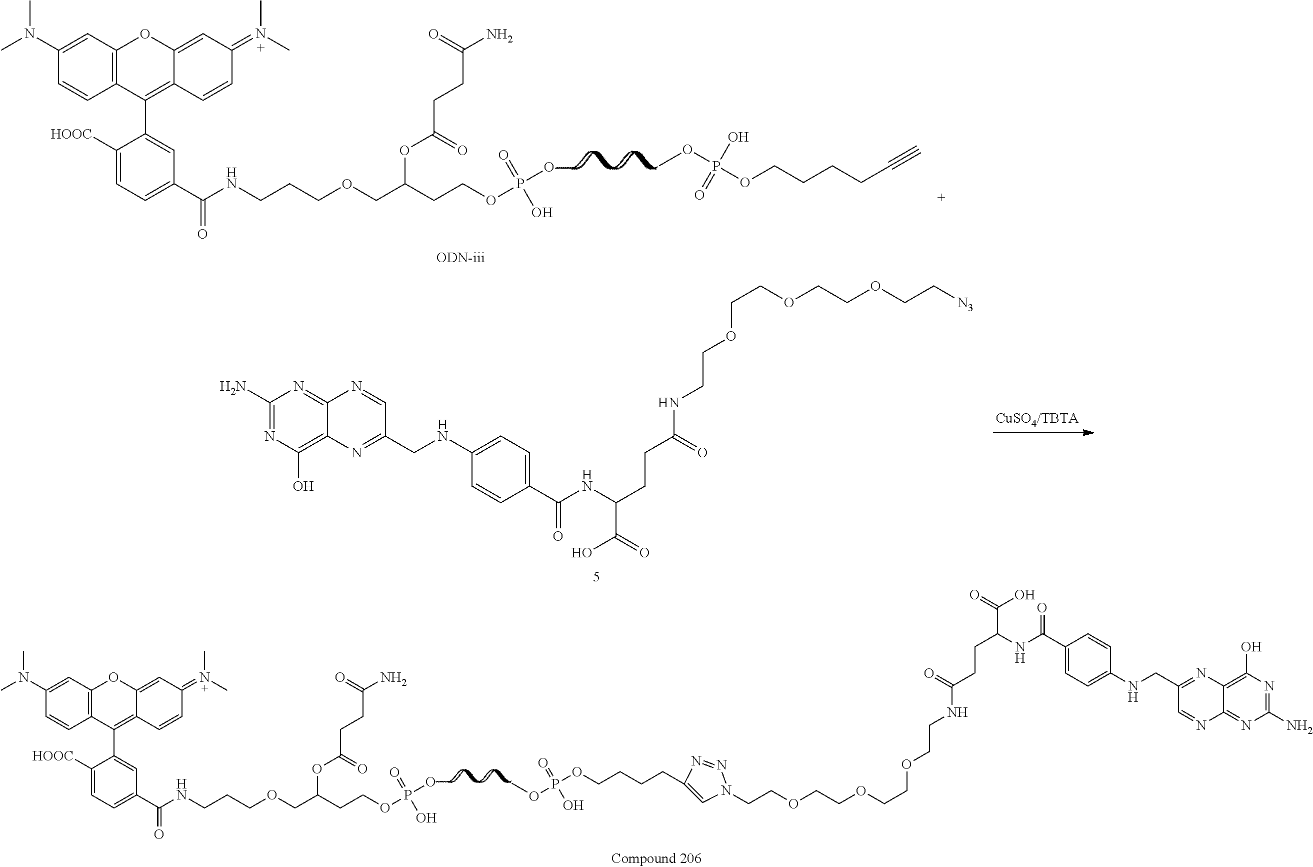

Folate azide 5 was prepared according to a previously published procedure.³ ODN-iii (150 nmol) was dissolved in 160 μl MQ water, followed by the addition of compound 5 (1.5 μmol), ascorbic acid (20 μl, 0.9 μmol), TEAA buffer (40 μl, 2 M, pH=7), and DMSO (200 μL). After degassing with argon, Cu-TBTA (80 μL, 0.9 μmol) was added, and the mixture was stirred for 12 h. The product was purified using RP-HPLC to afford Compound 206. MALDI-TOF MS (m/z): calcd. 9940, found 9941.

### OmpC Construction and Expression

OmpC construction. E. coli outer membrane protein C (OmpC) was isolated by PCR, amplified from E. coli ASKA library and cloned into pET21 using RF cloning OmpC_FpET21: TTTGTTTAACTTTAAGAAGGAGATATACATATGAAAGTTAAAGTACTGTCCCTC (SEQ ID No.: 11) and OmpC_RpET21: TTCCTTTCGGGCTTTGTTAGCAGCCGG ATCTTAGAACTGGTAAACCAGACCC (SEQ ID No.: 12). The resulting plasmid was a His-tag less construct. Polyhistidine-linker sequences were inserted in the predicted 7^{th} loop of the OmpC. OmpC-(6His)₁ contains 11 amino acid (Aa) sequence: SAGHHHHHHGT (SEQ ID No.: 13) was constructed by Inverse PCR using the following 2 primers: OmpC_His1 F:CATCATCACCATGGTACCTCTAAAGGTAAAAACCTGGGTCGTGGCTAC (SEQ ID No.: 14), and OmpC_His1R: ATGGTGATGATGATGATGACCCGCGGAGGTAC CATGGTGATGATGGTGATGACCCGCGGA (SEQ ID No.: 15). The resulting plasmid served as a template for introducing a second His-linker to obtain OmpC-(6His)₂ 22 Aa sequence: SAGHHHHHHGTSAGHHHHHHGT (SEQ ID No.: 16) by using the following 2 primers: OmpC_His2FInverse: CACCATCACGGTACCTCTAAAGGTAAAAAC CTGGGTCGTG (SEQ ID No.: 17) and OmpC_His2RInverse: GTGATGGTGACCC GCGGAGGTACCATGGTGATGATGGTGATG (SEQ ID No.: 18). An additional third His-linker was introduced to OmpC-(6His)₂ by using the following 2 primers: OmpC_His3FInverse: CATCATCATGGTACCTCTAAAGGTAAAAACCTGGGTCGTG (SEQ ID No.: 19) and OmpC_His3RInverse: ATGATGATGACCCGCG GAGGTACCGTGATGGTGGTGATGGTG (SEQ ID No.: 20). The resulting construct OmpC-(6His)₃ contains 33 Aa His-linker: SAGHHHHHHGTSAGHHHHHHGT SAGHHHHHHGT (SEQ ID No.: 21) in the same position at the predicted 7^{th} loop of OmpC. For the Inverse PCR cloning reactions one primer of each set of primers had to be phosphorylated.

Purification of OmpC. The expression of OmpC was tested in the whole cell extracts (WCE) and in the membrane fraction. Cultures expressing OmpC, and His-OmpC were harvested, resuspended in Na₂HPO₄ (10 mM, pH 7.3) and lyzed by sonication. A sample from each culture was analyzed by SDS-PAGE for the expression of OmpC in the WCE. Following sonication, the supernatant was separated by centrifugation at 13800 g for 10 min. The membrane fraction was recovered by centrifugation of the supernatant at 13800 g for 30 min., resuspended in 10 mM Na₂HPO₄, pH 7.3, 2% Triton X-100 and incubated at 37° C. for 30 min. The insoluble fraction was recovered by centrifugation at 13800 g for 30 min., washed and resuspended in 10 mM Na₂HPO4 pH 7.3. Proteins from the membrane fractions were analyzed by SDS-PAGE.

### Oligonucleotides

The oligonucleotides used in the experiments are detailed in Table 1.

TABLE 1
Oligonucleotides (ODNs)

| | | SEQ |
|---|---|---|
| | | ID |
| Description | Sequence | No. |
| Compound 100 | 5′GCGGCGAGGCAGC3′ | 1 |
| Compound 101 | 3′ATCCTAGTCCGTCGATACTGCACTG5′ | 2 |
| ODN-1 and | 3′ATCCTAGTCCGTCGATACT5′ | 3 |
| Compound 102 | | |
| Compound 103 | 3′GATGACAGCTAGCAGATCAACATGG5′ | 4 |
| Compound 104 | 3′CGCGCGAAAAAAAAAAAAGCAACGC5′ | 5 |
| Compound 200 | 5′TAGGATCAGGCAGCTATGACGTGAC3′ | 6 |
| Compound 201 | 3′CAGTGCAGTATCGACGGACTAGGAT5′ | 7 |
| Compound 202 | 3′CAGTGCAGTATCGACGGACTAGGAT5′ | 7 |
| and FAM-ODN-2 | | |
| Compound 203 | 3′CCATGTTGATCTGCTAGCTGTCATC5′ | 8 |
| Compound 204 | 3′GCGTTGCTTTTTTTTTTTTCGCGCG5′ | 9 |
| Compound 205 | 3′CAGTGCAGTATCGACGGACTAGGAT5′ | 7 |
| Compound 206 | 3′CAGTGCAGTATCGACGGACTAGGAT5′ | 7 |
| Compound 207 | 3′CAGTGCAGTATCGACGGACTAGGAT5′ | 7 |
| ODN-3 | 5′GTCACGTCATAGCTGCCTGATCCTA3′ | 10 |

### Bacterial Strains and Growth Conditions

E. coli K-12 strain KRX (Promega) was used for protein expression. Transformed bacteria with the different OmpC constructs (OmpC or His-OmpC) were cultured to saturation in LB medium supplemented with 100 μg/ml of ampicillin at 30° C. 40 μl of the pre-cultured cells were then diluted into 4 ml of fresh LB medium supplemented with ampicillin, and incubated until the OD₆₀₀ reaches˜0.6. Protein expression was then induced by the addition of 0.1% Rhamnose and 20 μM isopropyl-b-D-1-thiogalactopyranoside (IPTG) and cultures were allowed to grow at 30° C. for 18 h.

General Procedure for Decorating Bacteria with the Oligonucleotides

The bacterial cells (OmpC or His-OmpC) were collected by centrifugation at 6000 g for 4 min. The pellet was washed twice with PBS×1 buffer and resuspended in the same buffer to an OD₆₀₀ of 0.3. To a 100 μl sample of the bacteria suspension, a preincubated sample of DNA (500 nM) and NiCl₂ (2.5 μM) was added, and the cells were incubated at room temperature for 1 h. Then the bacterial sample were washed twice with PBS, resuspended in 100 μl PBS and placed on a glass-bottom dish (P35G-1.5-14-C; MatTek) precoated with poly-1-lysine (Sigma Aldrich) and left to adhere for 1 h. Finally, the wells were washed vigorously with PBS three times and imaged using an Olympus IX51 fluorescent microscope. The samples were imaged using 60× or 100× objective lenses.

Treatment of the Modified Bacteria with EDTA

Bacterial samples decorated with Compound 100 were incubated with various concentration of EDTA (0, 5, 10 mM) for 1 h. Cells were then collected (6,000 g, 4 min) and washed twice with 200 μl PBS buffer. Cells were resuspended in 100 μl PBS buffer and added to poly-l-lysine-coated slides for imaging.

### Flow Cytometry

Bacteria were decorated with Compound 101 according to the procedure described above. The samples were analyzed using BD FACS Aria Fusion instrument (BD Biosciences, San Jose, Calif., USA) equipped with 488 nm (blue), 561 nm (green), and 640 nm (red) lasers. Sorting was performed using a 100-μm nozzle equipped with BD FACS Diva software v8.0.1 (BD Biosciences). Data was analyzed using FlowJo software.

### Bacterial Cell Growth

His-OmpC bacteria decorated with Compound 101 was incubated for 30 min in M9 minimal medium containing 2% glucose. The sample was spun down at 6,000 g for 2 min and the supernatant was discarded. After washing the pellet with M9 minimal medium, the cells were diluted to OD₆₀₀=0.05 in M9 medium in a 96-well plate. Growth kinetics was monitored by recording OD₆₀₀ under shaking at 30° C. for 24h. Bacteria expressing His-OmpC was used as a control. The ability of the modified His-tagged bacteria to grow and divide was also demonstrated using fluorescence microscopy. For these experiments, the bacteria were prepared using a similar procedure. After diluting the sample to OD₆₀₀=0.3, it was allowed to grow at 30° C. 100 μl samples were withdrawn at different time intervals and plated on poly-l-lysine-coated glass bottom dishes and imaged by fluorescent microscopy.

### Introducing Posttranslational Modifications' to the Bacteria

Bacterial cells were decorated with ODN-1 according to the procedure described above. After washing the sample with PBS, the following ODNs were added sequentially: Compound 200, ODN-3, Compound 201, ODN-3, Compound 202, and ODN-3. After each incubation step, cells were washed twice with PBS and a sample was taken for imaging before the addition of the subsequent strand. Fluorescently labeled ODN-2 strands were added at a concentration of 500 nM and incubated for 30 min, while ODN-3 strand was added at a concentration of 2 μM and incubated for 2 h.

### Mixed Population of Bacteria

Three samples of His-OmpC bacteria (100 μl each) were separately labeled with Compound 102, Compound 103, or Compound 104. Each sample was washed twice with PBS. Then, an equal ratio (30 μl each) of the three samples were combined and Compound 202, Compound 203 and Compound 204 (500 nM) were added to the mixture and incubated for 10 min. The bacterial cells were centrifuged at 6,000 g for 2 min, washed twice with PBS and imaged by fluorescent microscopy using 488, 561, and 647 nm excitation lasers and 488/50, 610/60, and 685/50 emission filters. For flow cytometry analysis, the samples were not washed after addition of ODN-2 strands.

### Bacteria-Streptavidin Interaction

His-tagged bacterial cells were decorated with a duplex consisting of ODN-1 and Compound 205 duplex according to a similar procedure described above. For binding with streptavidin, cells were incubated with Alexa-647 streptavidin conjugate (500 nM) in PBS×1 for 1 h, and after washing twice with PBS were imaged by fluorescent microscopy. The fluorescent signal was abolished when bacterial cells were treated with ODN-3 (3 μM) for 1 h. The control experiment was performed similarly using bacteria decorated with a duplex containing ODN-1 and the complementary strand.

### Bacteria-KB Cell Interaction

KB cells were maintained in folate-depleted RPMI supplemented with 10% fetal bovine serum (FBS), 1% L-glutamine, and 1% penicillin/streptomycin. Cells (12,500 cells/well) were seeded onto glass bottom culture dishes (Mattek) and allowed to adhere overnight. Cells were then washed twice with PBS and incubated with 100 μl His-tagged bacteria decorated with ODN-1: Compound 206 duplex for 30 min. The medium was removed and cells were rinsed three times with PBS. Cells were then imaged using a fluorescence microscope and a 60× objective lens. A control experiment was performed similarly using bacteria decorated with a duplex lacking the folate moiety (ODN-1 and ODN-iii). To show the reversibility of interaction, the bacteria bound KB cells were incubated with ODN-3 (5 PM) for 15 min. After washing twice with PBS buffer, cells were imaged again.

### Adhesion to the Solid Support

The gold substrates were prepared by electron-beam evaporation of an adhesion layer of chromium (3 nm), followed by a 20 nm layer of gold (99.99% purity) onto high precision cover glasses (170±5 μm, Marienfeld-Superior, Germany). A solution of (11-mercaptoundecyl)tetra(ethylene glycol)⁹ (2 mM in ethanol) were added to the gold coated substrates and incubated for 2 h. After removing the solution, the slides were washed four times with ethanol. Bacteria samples decorated with a duplex consisting of Compound 102 and Compound 207 were washed twice with PBS, resuspended in 100 μl phosphate buffer (pH=3.8), and then incubated on gold surfaces for 15 min. The solution containing bacteria was removed, and the slides were rinsed three times with PBS, and twice with water. Finally, they were imaged using an Olympus IX51 microscope.

### Super-Resolution Microscopy

Super-resolution images were collected on a Vutara SR200 STORM (Bruker) microscope based on the single-molecule localization biplane technology. His-tagged bacteria was decorated with ODN-1:Compound 201 duplex according to the procedure described in above. The bacteria were imaged using 647 nm excitation laser and 405 nm activation laser in an imaging buffer composed of 5 mM cysteamine, oxygen scavengers (7 μM glucose oxidase and 56 nM catalase) in 50 mM Tris, 10 mM NaCl and 10% glucose at pH 8.0. Images were recorded using a 60× NA 1.2 water immersion objective (Olympus) and Evolve 512 EMCCD camera (Photometrics) with gain set at 50, frame rate at 50 Hz, and maximal power of 647 and 405 nm lasers set at 6 and 0.05 kW/cm², respectively. Total number of frames acquired was 8000. Data was analyzed by the Vutara SRX software.

### Example 2—Design Principles of a Dynamic Artificial Receptor System

Objective: To produce an artificial receptor fulfilling the following requirements: (1) the artificial receptor is easily modifiable by molecular signals in their environment, (2) the artificial receptor is capable of attaching different bioactive molecules, labeling molecules, and synthetic agents, (3) the artificial receptor does not perturb desirable cell functions, (4) the artificial receptor can be reversibly modified.

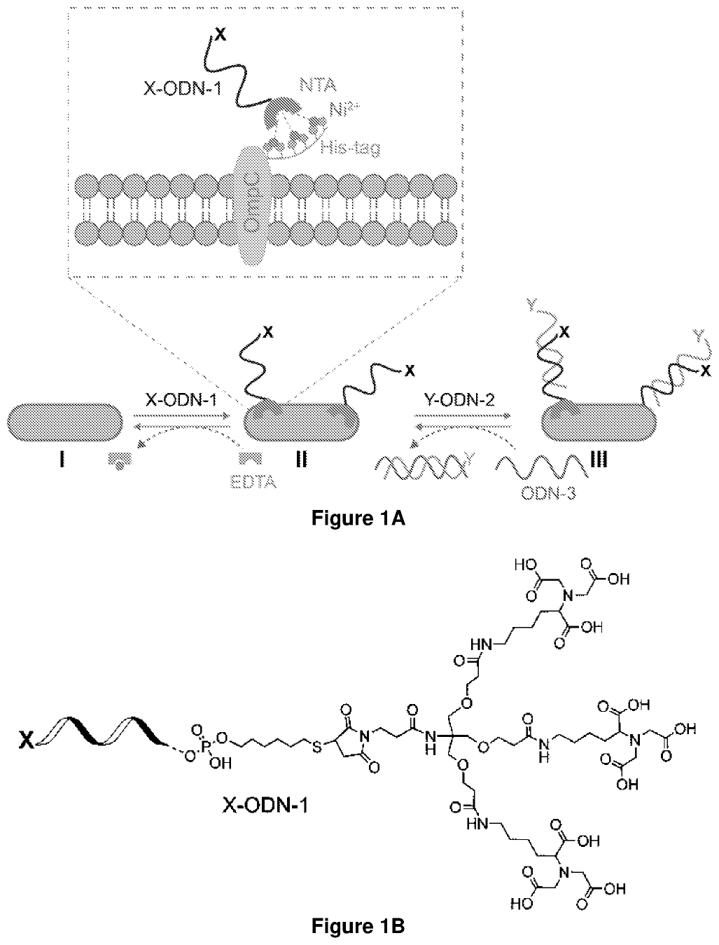

FIG. 1A shows the design and operation principles of an embodiment of the synthetic receptor system presented herein. The system comprises: A first polypeptide, said polypeptide comprising a membranal anchoring domain and an extracellular binding domain. In the examples shown herein, the membranal anchoring domain used is outer membrane protein C (OmpC) and the extracellular binding domain is hexa-histidine tag (His-tag). The first compound is sometimes termed His-OmpC in the Examples.

The first compound, comprises a first oligonucleotide (ODN-1) bound to a binder, said binder comprises affinity to said extracellular binding domain. The first compound is sometimes termed X-ODN-1 in the Examples, wherein ODN-1 denotes the first oligonucleotide, and X denotes an optional labeling moiety. In the examples shown herein, the binder is a three nitrilo acetic acid (Tri-NTA) conjugate, which binds His-tag. FIG. 1B shows an embodiment of X-ODN-1.

The second compound comprises a second oligonucleotide (ODN-2) bound to a synthetic agent on its end. The second compound is sometimes termed Y-ODN-2 in the Examples, wherein ODN-2 denotes the second oligonucleotide, and Y denotes the synthetic agent on its end. The oligonucleotide ODN-2 is complementary to the first oligonucleotide ODN-1. However, Y-ODN-2 bears also a short overhang region, termed a toe-hold region. Such toe-hold region can be used to initiate strand displacement and detachment of Y-ODN-2 from X-ODN-1 by an oligonucleotide complementary to the whole ODN-2 oligonucleotide.

The system optionally comprises a third compound, comprising a third oligonucleotide (ODN-3). The oligonucleotide ODN-3 is complementary to the whole ODN-2 sequence, i.e., both to the toe-hold region and to the region bound to ODN-1. Cells can be optionally incubated with ODN-3, which produces strand displacement. In a first step, ODN-3 binds to Y-ODN-2 toe hold region. In a second step, ODN-3 competes with ODN-1 for binding with ODN-2, until eventually it detaches Y-ODN-2 from X-ODN-1.

The artificial receptor system described above was used for decorating a cell surface according to at least two approaches. In the first approach, cells expressing His-OmpC were incubated with X-ODN-1 in the presence of Ni (II) (FIG. 1, steps I and II). X-ODN-1 was efficiently bound to His-OmpC in such conditions. The effect of the synthetic agent was terminated by detaching X-ODN-1 from His-OmpC, for example by incubating the cells with a Ni (II) chelator as EDTA.

In the second approach, cells expressing His-OmpC were first incubated with X-ODN-1 in the presence of Ni (II) (FIG. 1, steps I and II). Then, cells were incubated with Y-ODN-2, which bound to X-ODN-1 (FIG. 1, steps III). Optionally, addition of ODN-3 terminated the effect of the synthetic agent of Y-ODN-2 (FIG. 1, steps III and II).

The artificial receptor system developed and disclosed herein present a number of advantages. First, the receptors are non-covalently anchored to the cellular membrane. Such non-covalent anchoring allows controlling the number of receptors on the cell membrane and surface by external molecular signals (e.g., X-ODN-1, EDTA, Y-ODN-2, and ODN-3). Second, the anchoring domain of the receptors is stably inserted into the cell membrane, and an extracellular domain can bind different synthetic agents. Thus, different synthetic agents can be bound to the extracellular domain without re-engineering the cells. Third, the anchoring domain has a minimal size and is present only at specific locations on the bacteria membrane. Thus, the anchoring domain does not perturb cellular function. Fourth, the synthetic receptors can be to reversible modified. This allows dynamically altering their structure while they are attached to the bacterial membrane, resembling post-translational modifications that occur on natural receptors.

### Example 3—Decorating Bacteria with Artificial Receptors and Controlling the Receptors Functioning

Objective: To decorate bacterial membranes with an artificial receptor.

Methods: His-tagged OmpC was expressed in E. coli, which was then incubated with an X-ODN-1 appended either with a Cy5 dye or TAMRA (Compounds 100-101) in the presence of nickel ions and EDTA. Methods and protocols are detailed in Example 1.

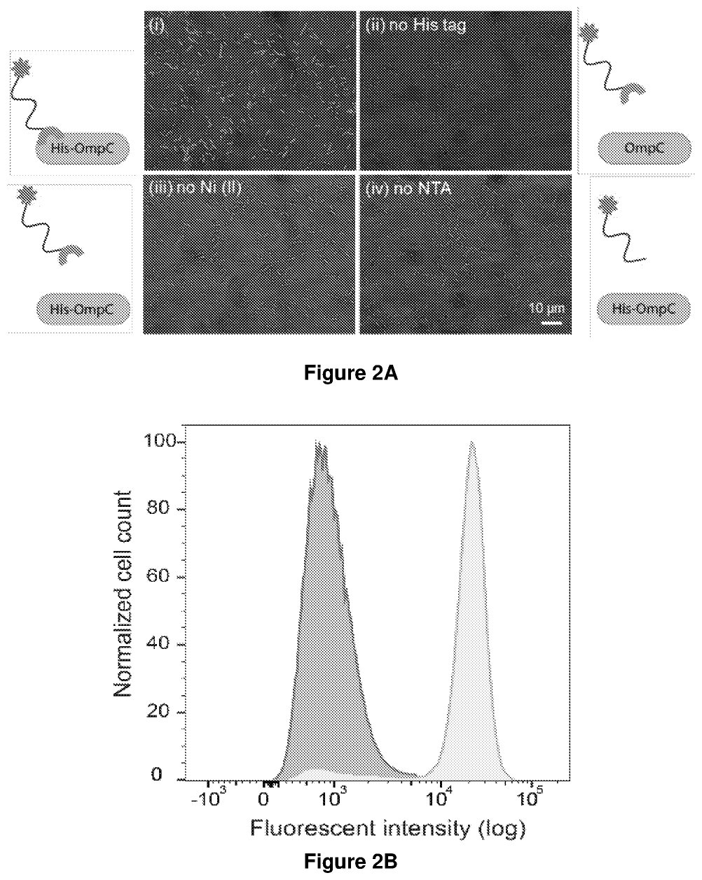

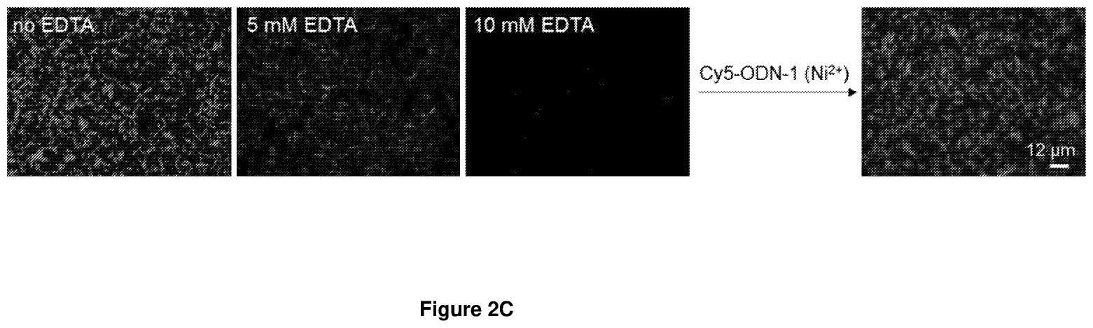

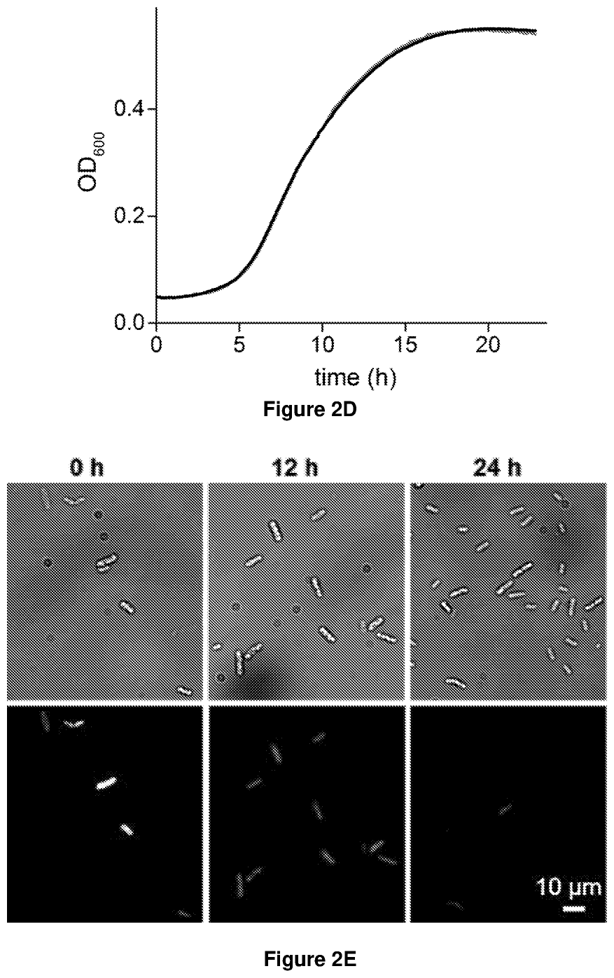

Results: Fluorescence imaging revealed that His-tagged OmpC engineered bacteria incubated with Compound 100 were successfully decorated with the Cy5 fluorophore (FIG. 2A, i). To confirm that the labeling did not result from a non-specific interaction between Compound 100 and the bacteria surface, Compound 100 was also incubated with native bacteria lacking His-OmpC (FIG. 2A, ii), as well as with the His-tagged bacteria in the absence of nickel ions (FIG. 2A, iii). Additionally, His-tagged bacteria was incubated with a Cy5-labeled ODN lacking a tri-NTA group (FIG. 2A, iv). No fluorescence was observed in any of these controls, confirming the selectivity of ODN-1 to membrane bound His-tags.

The selectivity and degree of labeling were further analyzed by flow cytometry. 90.9% of His-tagged modified bacteria and 1% of native bacteria were labeled by Cy5 (FIG. 2B).

The ability of the system to control the activity levels of the artificial receptors by external signals was further tested. Bacteria were exposed to increased concentrations of EDTA, which resulted in a decrease in surface coverage with Compound 100. 10 mM of EDTA completely removed Compound 100 from the cell surface. Detached Compound 100 could be washed from the medium and bacteria could be re-decorated with other molecules (FIG. 2C).

To confirm that attachment of X-ODN-1 does not affect the ability of the bacteria to grow and divide, the growth of TAMRA-ODN-1 (Compound 101) decorated bacteria was measured by optical density (OD) and compared to that of bare His-tagged bacteria. The growth kinetic curves were not affected by Compound 101 binding (FIG. 2D) indicating that the biomimetic cellular surface protein system does not affect cell division and survival.

The ability of Compound 101 decorated bacteria to grow and divide was further demonstrated using fluorescence microscopy. Fluorescence microscopy revealed that the number of Compound 101 labeled cells increased with time, but that the fluorescence recorded in each cell decreased (FIG. 2E). These results were interpreted as a consequence of the Compound 101 molecules being divided between the daughter cells in each division.

### Example 4—Reversible Modification of Membrane-Bound Synthetic Receptors Using Complementary Strands

Objective: To reversibly modify the synthetic receptors by external molecules.

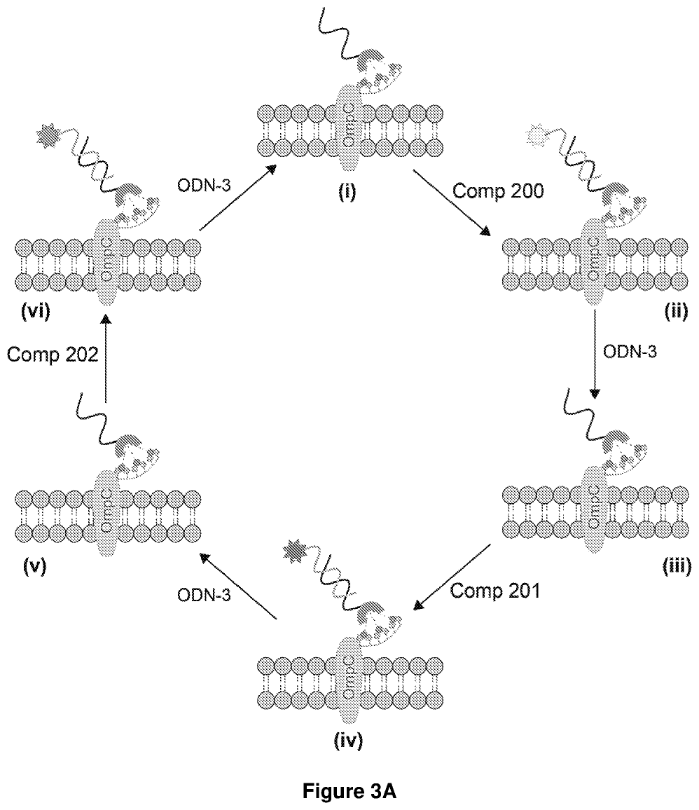

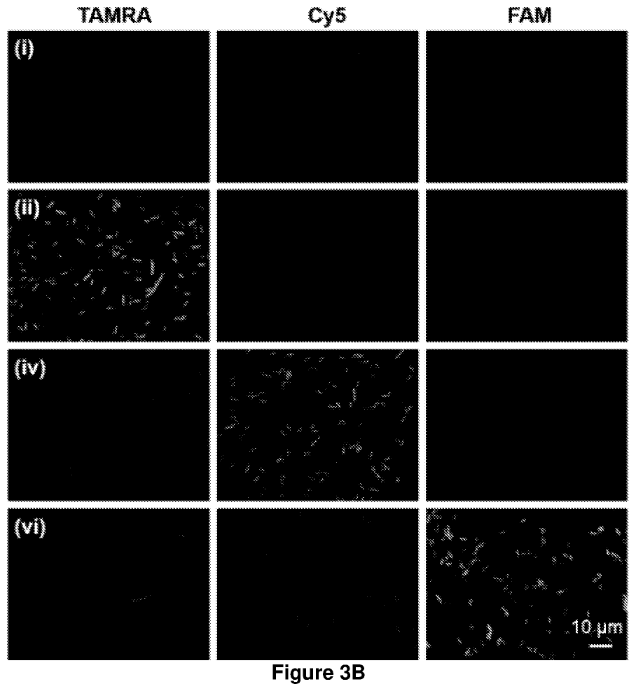

Methods: FIG. 3A schematically illustrates the experiments detailed herein. E. Coli ectopically expressing His-OmpC were first incubated with oligonucleotide X-ODN-1 (FIG. 3A, step (i)). Afterwards cells were incubated with a Compound 200, wherein ODN-2 is an oligonucleotide complementary to ODN-1 (FIG. 3A, step (ii)). Cells were then incubated with an ODN-3 oligonucleotide complementary to ODN-2 (FIG. 3A, step (iii)). Then cells were incubated with a Compound 201 (FIG. 3A, step (iv)). Next, cells were again incubated with an ODN-3 oligonucleotide (FIG. 3A, step (v)). Cells were finally incubated with a Compound 202 (FIG. 3A, step (vi)). Fluorescence was measured in all steps assessing the binding of TAMRA, Cy5, and FAM to the cell membranes.

Results: Fluorescence microscopy revealed the presence of the corresponding dye (TAMRA, Cy5, and FAM) after bacteria were incubated with it. Further, the fluorescent emission disappeared after each time bacteria were incubated ODN-3 (FIG. 3B).

### Example 5—Decorating Populations of Heterogenous Bacteria with Different Artificial Receptors

Objective: To create a mixed population of bacteria, where each subpopulation bears a different sequences of ODN-1 and is modified by a different X-ODN-2 molecule.

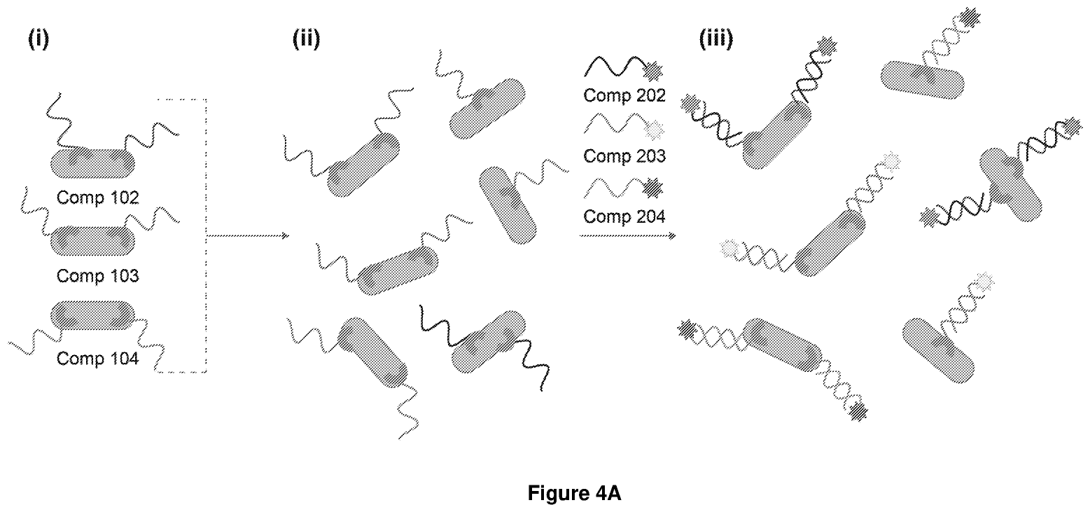

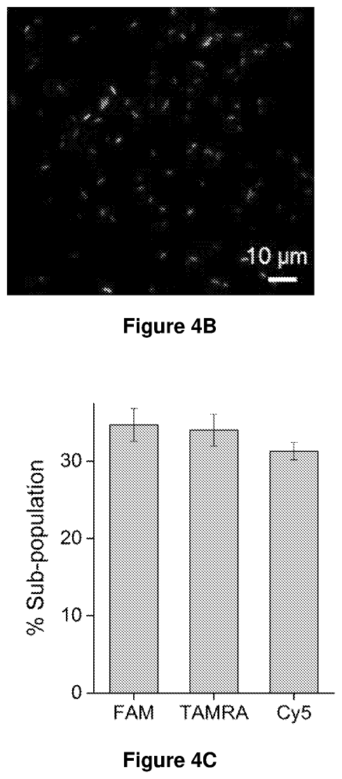

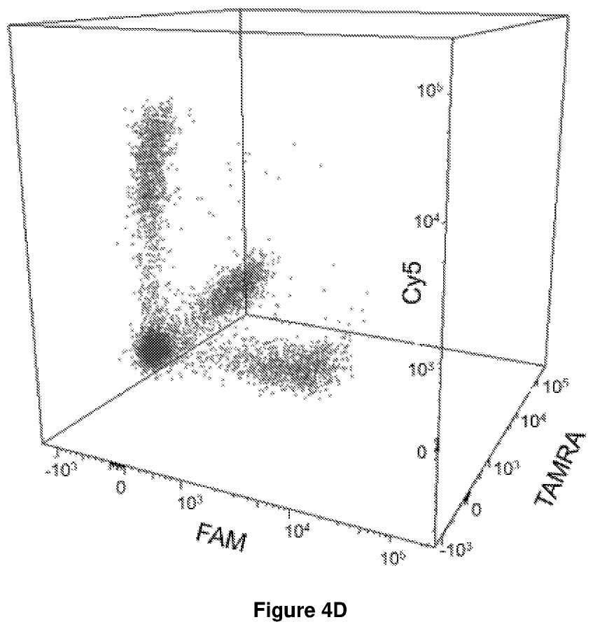

Methods: Three populations of His-tagged E. coli were incubated with three different types of ODN-1 (Compound 102, Compound 103, and Compound 104; Compound 102, 103 and 104 respectively), which bared the same tri-NTA types but differed in their oligonucleotide sequences. Then, the three samples were combined and incubated with a mixture of three types of dye-labeled ODN-2 (Compound 202, Compound 200, and Compound 201 respectively); each of which was complementary to only one of the bacteria-bound ODN-1s (FIG. 4A). Bacteria were then analyzed by fluorescent microscopy and FACS.

Results: Fluorescence microscopy (FIG. 4B) and FACS analysis (FIG. 4C) revealed the presence of three distinct groups of bacteria, each labeled with only one dye. Calculating the percentage of each population out of the total number of bacteria revealed a 1:1:1 ratio between the three sub-populations. (FIG. 4D) indicating that there is no strand swap between the three populations and that the sub-population modification occurs with very high selectivity.

Discussion: This experiment demonstrates a means to selectively label His-tagged proteins with different colors. Hence, one practical application that can be achieved with this approach is using the synthetic receptors to image specific proteins or cellular compartments in living cells. The advantage of using this method, over using other fluorescent probes that can bind and label short fusion peptides in living cells is the simplicity by which the fluorescent dye can be changed. Specifically, when DNA duplex-based fluorescent probes are used for live cell imaging there is no need to synthetize a new probe for each application. Instead, various different fluorescent dyes can be used for imaging, simply by preparing a wide range of fluorescently labeled ODN-2s from commercially available phosphoramidites and by using an automated DNA synthesizer.

### Example 6—Endowment of New Properties to Bacteria by Artificial Receptors

Objective: To endow bacteria with unnatural and potentially useful properties by using the artificial receptor system.

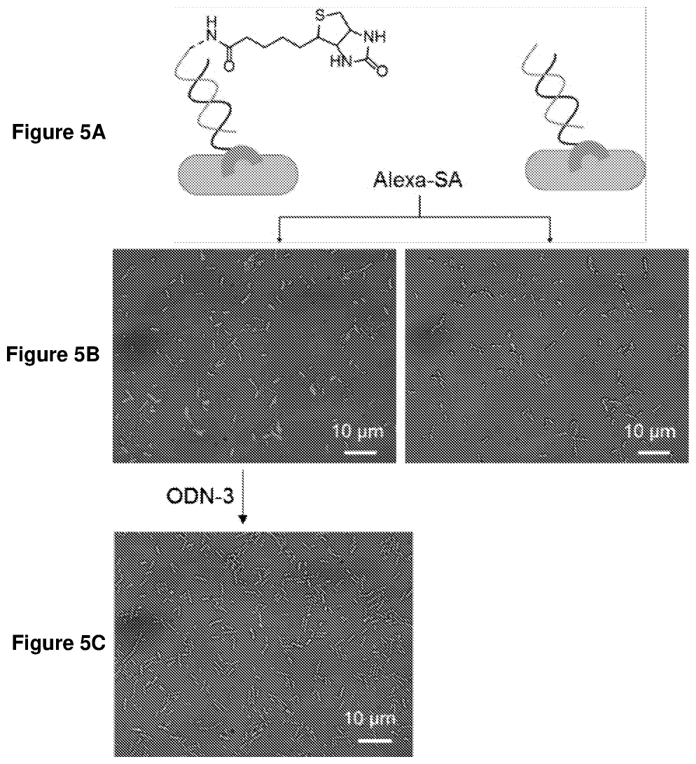

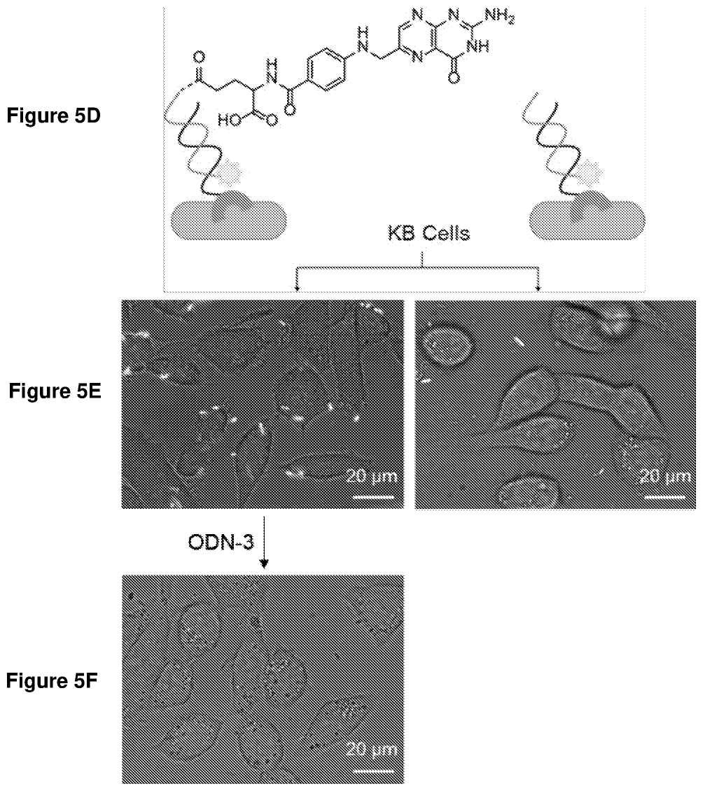

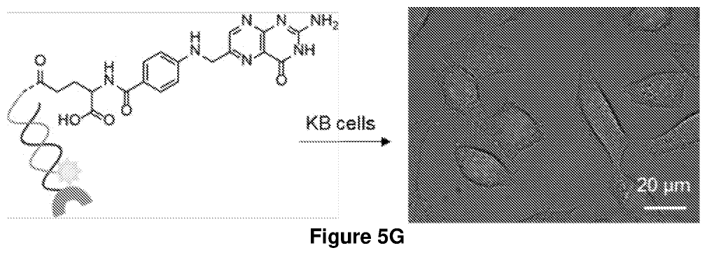

Methods: His-tagged E. coli were incubated with an ODN-1 molecule and afterwards with a biotin-ODN-2 molecule (Compound 205). Then, the cells were incubated with an Alexa 647-modified streptavidin (FIG. 5A). To verify specificity, the same experiment was performed with an ODN-2 molecule lacking biotin (FIG. 5A). Cells were then incubated with ODN-3 to detach ODN-2 from the cell membranes.

Results: Fluorescent microscopy revealed that bacteria became fluorescent only when Compound 205 was incorporated in the synthetic receptor (FIG. 5B), indicating specific binding of the protein to the bacterial membrane. The fluorescent signal disappeared when ODN-3 was added (FIG. 5C), indicating the reversibility of this process, and suggesting the possibility of regulating unnatural cell-protein-interactions using synthetic molecular signals as Compound 205 and ODN-3.

### Example 7—Induction of Unnatural Cell-Cell Interactions by Artificial Receptors

Objective: To test whether synthetic receptor-protein interactions can mediate unnatural cell-cell interactions in general, and interactions resembling bacterial-mammalian cell interactions in particular.

Methods: His-tagged bacteria were decorated with a DNA duplex containing Compound 101 and a folate-modified ODN-2 (compound 206). Then, bacteria were incubated with human epidermoid carcinoma KB cells overexpressing an extracellular folate receptor (FIG. 5D). As a control, KB cells were incubated with bacteria decorated with a similar TAMRA-labeled DNA duplex lacking the folate group (FIG. 5D). Cells were then incubated with ODN-3 to detach compound 206 from bacteria membranes.

Results: Fluorescent imaging revealed KB cells were labeled with glowing bacteria when incubated with compound 206 bound bacteria, but not with control bacteria (FIG. 5E). Incubation with ODN-3 fully detached compound 206 from the bacteria, thus releasing the bacteria from the KB cells (FIG. 5F).

Incubation of KB cells with the DNA duplex alone (without His-tagged bacteria) did not result in fluorescent cancer cell labeling (FIG. 5G). This observation indicates that the bacteria scaffold itself plays a critical role in the interaction of folate with the folate receptor. One contribution of the bacteria to effective cell labeling is an increased avidity, which results from multivalent interactions between natural folate receptors on the KB cell and the folate-modified DNA duplexes on the surface of E. Coli. The second contribution is that each bacterial cell is decorated with multiple fluorophores, leading to a bright fluorescent labeling and consequently, to sensitive detection.

Discussion: These experiments provide evidence that unnatural cell-cell interactions can be both induced and disrupted using a biomimetic receptor system that responds to external molecular signals, such as compound 206 and ODN-3, respectively.

These experiments also demonstrate the relevance of this study to cell-based therapy. Here it is shown the ability to program bacterial cells to target cancer cells with increased avidity and selectively, by using synthetic cell-surface receptors to guide therapeutic cells to their targets. Further, the disruption of bacteria-cancer cell interactions with ODN-3 suggests that this approach can be used as an antidote to this class of therapeutics.

### Example 8—Induction of Bacterial Adhesion to Abiotic Surfaces by Artificial Receptors

Objective: To test whether synthetic receptor can provide bacteria with the ability to interact selectively with solid substrates.

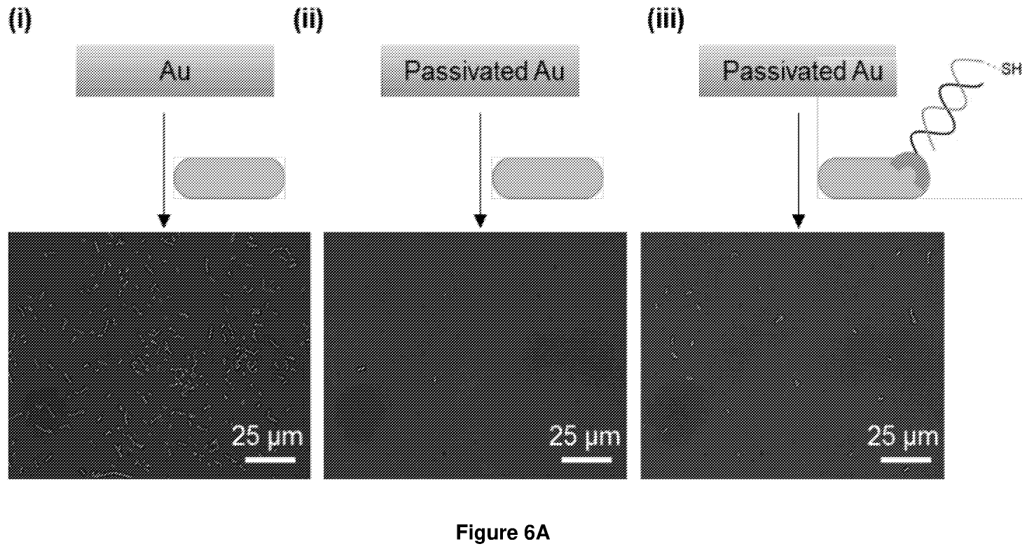

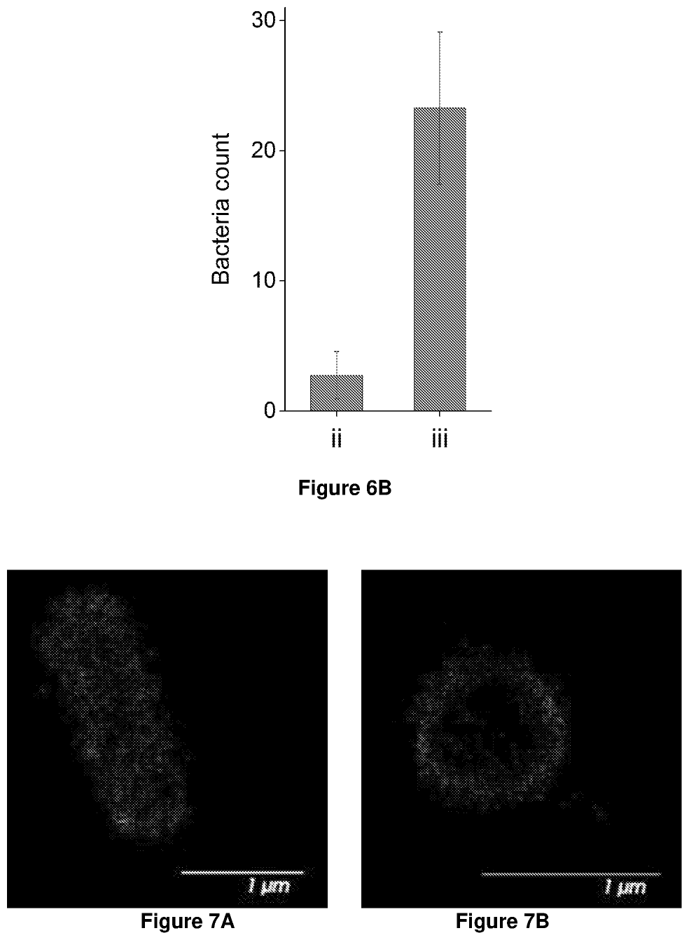

Methods: His-tagged bacteria were decorated with a duplex assembled from ODN-1 and HS-ODN-2 (Compound 207), namely, an ODN-2 that is appended with a thiol group. HS is known to have high affinity to gold. In the following step, unmodified His-tagged bacteria and thiol-modified His-tagged bacteria (FIG. 6A) were incubated with a gold substrate that was previously passivated with (11-mercaptoundecyl)tetra(ethylene glycol) to prevent non-specific bacterial adhesion. Gold surfaces were observed after 15 min incubation. Cells were then incubated with ODN-3 to detach Compound 207 from bacteria membranes.

Results: Microscopy revealed an increase of about 8.5-fold in the attachment of thiol-modified bacteria to the gold substrate compared with the control (FIG. 6B). This indicates that the ODN-1:Compound 207 duplex acts as an unnatural adhesin that can mediate specific binding of bacteria to solid support. The selectivity of these synthetic adhesin to gold was further demonstrated by incubating the thiol-modified bacteria with the gold substrate in the presence of ODN-3, which led to a significant decrease in the number of surface-bound His-tagged bacteria.

Discussion: In the context of biomimicry, disruption of adhesion owing to changes that occur on the synthetic receptors resembles the way post-translational modification of natural adhesins are used by bacteria to disrupt adhesion processes. The unnatural adhesins presented herein can be used to have a precise control of the way bacteria are attached to solid supports. For example, changing the length of the DNA linkers or attaching the modified bacteria to more complex DNA architectures (such as DNA Origami/nanotechnology type structures) on the surface may alter the binding properties of the bacteria. Further, the approach presented herein can be used to generate engineered living materials (ELMs) made of controlled bacterial assemblies.

### Example 9—Induction of Luminescence in Bacteria by Artificial Receptors

Background: Reversible switching of luminescence in response to the binding of cell surface proteins to extracellular molecular signals is a fundamental property of serval bacterial strains. A key principle underlying natural bacterial luminescence processes is the selective interaction between peptide autoinducers (AI) and their protein receptors, which enables them to trigger the emission of specific bacterial strains in complex biological mixtures. According to this invention, the ability to selectively label specific bacteria (modified with a unique ODN-1) in complex mixtures is described.

Objective: To control bacterial cell luminescence using biomimetic receptor systems (using super resolution microscopy).

Methods: Due to the small size of bacteria, super resolution (SR) microscopy was used to visualize E. Coli's membrane with super resolution (SR). This was achieved by combining ODN-1 with a commercially available ODN-2 (Cy5-ODN-2; Compound 201) bearing a Cy5 dye, which is compatible with stochastic optical reconstruction microscopy (STORM). SR images of individual bacteria revealed that DNA duplex-based label clearly outlines the bacterial cell's borders (FIG. 7A). Imaging of the transverse cut of the bacteria confirms that only the outer membrane of the bacteria is labeled, namely, that the synthetic receptors are exposed on the bacterial surface and are not internalized (FIG. 7B).

### Example 10—Discussion

The Examples disclosed above show a number of unexpected advantages as shown in the following examples: 1) The His-OmpC molecule can be stably expressed in E. coli. 2) The hexa-histidine moiety does not perturb the function of cell or of the synthetic agent due to its small size. 3) The His-tag can be efficiently targeted by NTA-Ni (II) complexes, including complexes of ODN-NTA conjugates. 4) The binding of His-OmpC to X-ODN-1 can be efficiently released by incubating the cells with a Ni (II) chelator, as EDTA. 5) The use of Y-ODN-2 circumvents the complexity of synthesizing the oligonucleotide X-ODN-1 which is attached on one end to the Tri-NTA moiety, and on the other to a synthetic agent. 6) The activity of the synthetic agent of Y-ODN-2 can be effectively terminated by incubating the cells with ODN-3.

The advantages of using ODN-small molecule conjugates as synthetic protein binders include the ability to precisely control the orientation, distances and valency of their binding units, as well as the ability to dynamically change their structure, which provides a means to regulate protein functions in real time. The Examples provided herein show that when synthetic proteins binders of this class are attached to cell's surfaces, their regulatory effect can be extended from the protein level to the cellular level. Specifically, on the cell' membrane such systems can act as artificial cell surface receptors that can be reversibly modified and hence, can provide the cells with ‘programmable’ properties. In this model system, metal coordination and DNA-hybridization were used to direct the formation of artificial receptors on a short peptide tag fused to an outer membrane protein on the surface of E. coli. Owing to the high selectivity and reversibility of the self-assembly processes, a biomimetic cell surface receptor system with unique features was obtained. For example, the ability to control reversibly the type of membrane-bound receptors and their local concentration levels with external molecular signals demonstrates the possibility of imitating dynamic processes that occur of cell surface proteins, such as changes in their expression level or post-translational modification. It was also shown that these changes can provide the bacteria with new properties such as an ability to glow with different colors, adhere to surfaces, and interact with proteins or cells; properties that may eventually be used in developing cell imaging methods, living materials and devices, or cell-based therapeutics, respectively. In light of these potential applications, the studies presented herein guide the development of additional biomimetic cell surface receptors, with which living cells could be ‘programed’ to preform diverse sets of functions.

1-137. (canceled)

138. A system comprising:
a. a recombinant cell ectopically expressing a polypeptide, wherein said polypeptide comprises a membranal anchoring domain and an extracellular binding domain,
b. a first compound comprising a first oligonucleotide (ODN-1) covalently bound to a binder, either directly or through a first linker, said binder comprising affinity to said extracellular binding domain, and
c. a second compound comprising a second oligonucleotide (ODN-2) covalently bound to a synthetic agent, either directly or through a second linker, wherein said second oligonucleotide is complementary to said first oligonucleotide.

139. The system of claim 13838,
wherein said polypeptide is a cell surface protein (CSP),
wherein the system does not perturb said cell's function,
wherein said system can be reversibly modified,
wherein said recombinant cell is selected from: eukaryotes, prokaryotes, mammalian cells, plant cells, human cells, and bacteria,
wherein said membranal anchoring domain comprises a transmembranal protein or a part of it, an artificial polypeptide, or a combination thereof,
wherein said binder comprises a His-tag specific binder,
said extracellular domain comprises an affinity tag,
or any combination thereof.

140. The system of claim 139, wherein
said transmembranal protein comprises an outer membrane protein C (OmpC); receptor tyrosine kinases (RTKs): Ion channel linked receptors; Enzyme-linked receptors; C protein-coupled receptors or any combination thereof,
said affinity tag comprises a poly-histidine peptide (6×-His-tag, 10×-His-tag, His-tag), a tetra cysteine peptide (CCPGCC, TC tag),
or a combination thereof.

141. The system of claim Error! Reference source not found.139,
wherein said His-tag specific binder comprises a moiety represented by the structure of formula E: 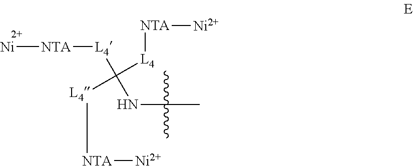
wherein
L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof;
formula E(a): 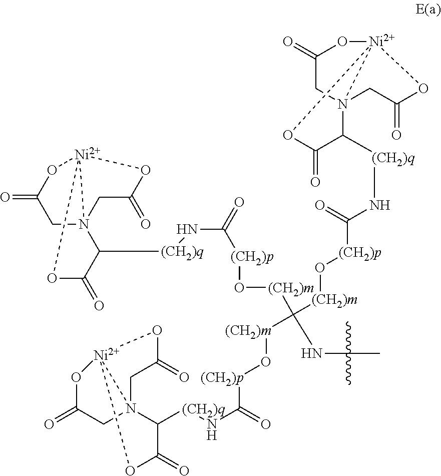
wherein
m, p and q are each independently an integer number between 1 and 8; or formula E(b):

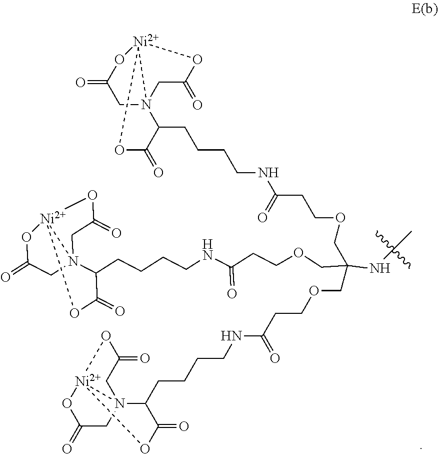

142. The system claim 13838, wherein said first linker comprises at least one polyethyleneglycol (PEG) moiety, at least one phosphate moiety, at least one thioalkyl moiety or any combination thereof;
or said first linker is represented by the following formula:
—[(CH₂O)ₖ—PO₃H]ₗ—(CH₂)_{w}—S—
wherein
k and l are each independently an integer number between 0 and 10; and
w is an integer number between 1 and 10.

143. The system of claim 13838, wherein said first compound further comprises a labeling moiety.

144. The system of claim 143, wherein said labeling moiety is bound to the 3′ end or to the 5′ end of said first oligonucleotide, directly or through a third linker; and/or
wherein said labeling moiety comprises a fluorescent dye.

145. The system of claim 144, wherein said fluorescent dye is selected from a group comprising dansyl, fluorescein (6-FAM), FAM, cyanine dyes (e.g. Cy3, Cy5) sulfoindocyanine, nile red, rhodamine, perylene, fluorenyl, coumarin, 7-methoxycoumarin (Ma), dabcyl, NBD, Nile blue, TAMRA, BODIPY, FITC or derivative thereof.

146. The system of claim 138, wherein said first compound is represented by the structure of the nickel complexes of the following compounds:

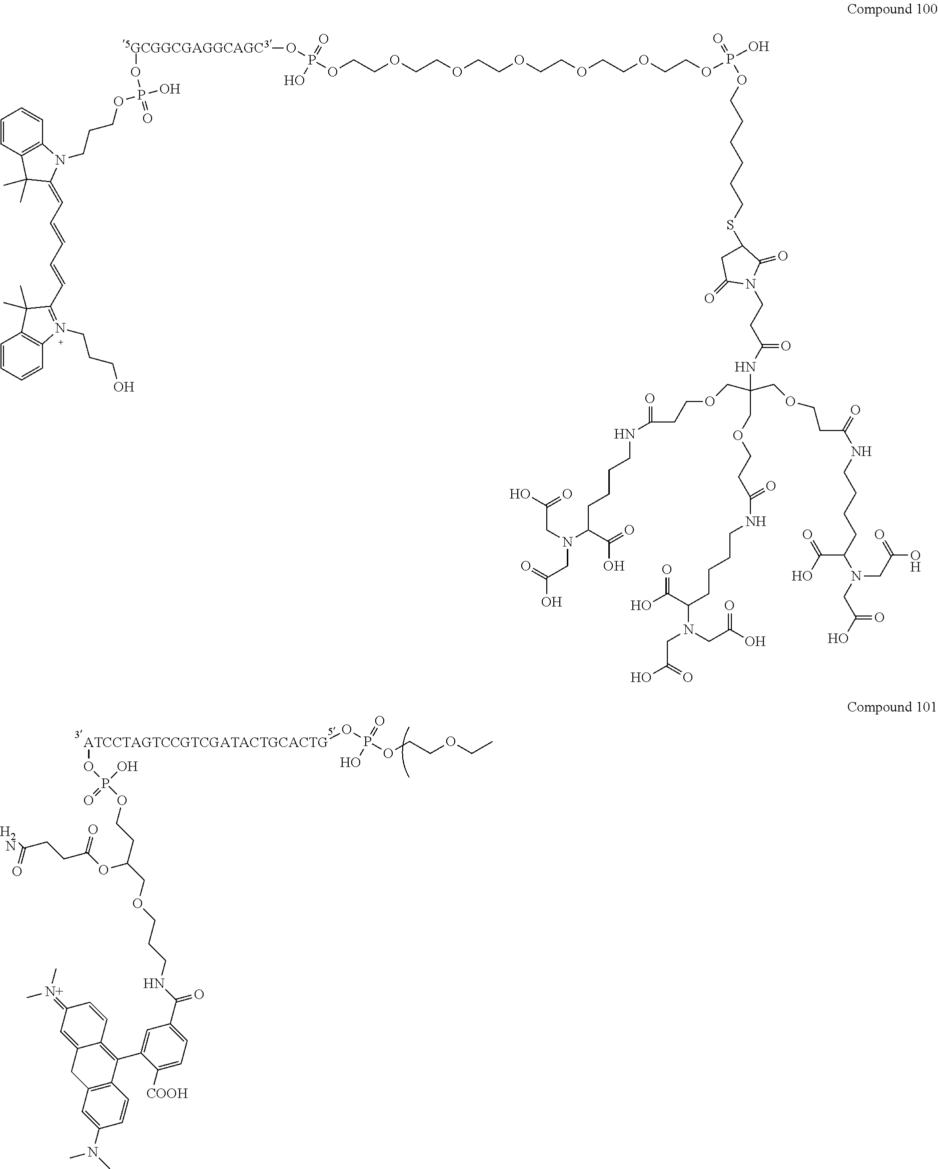

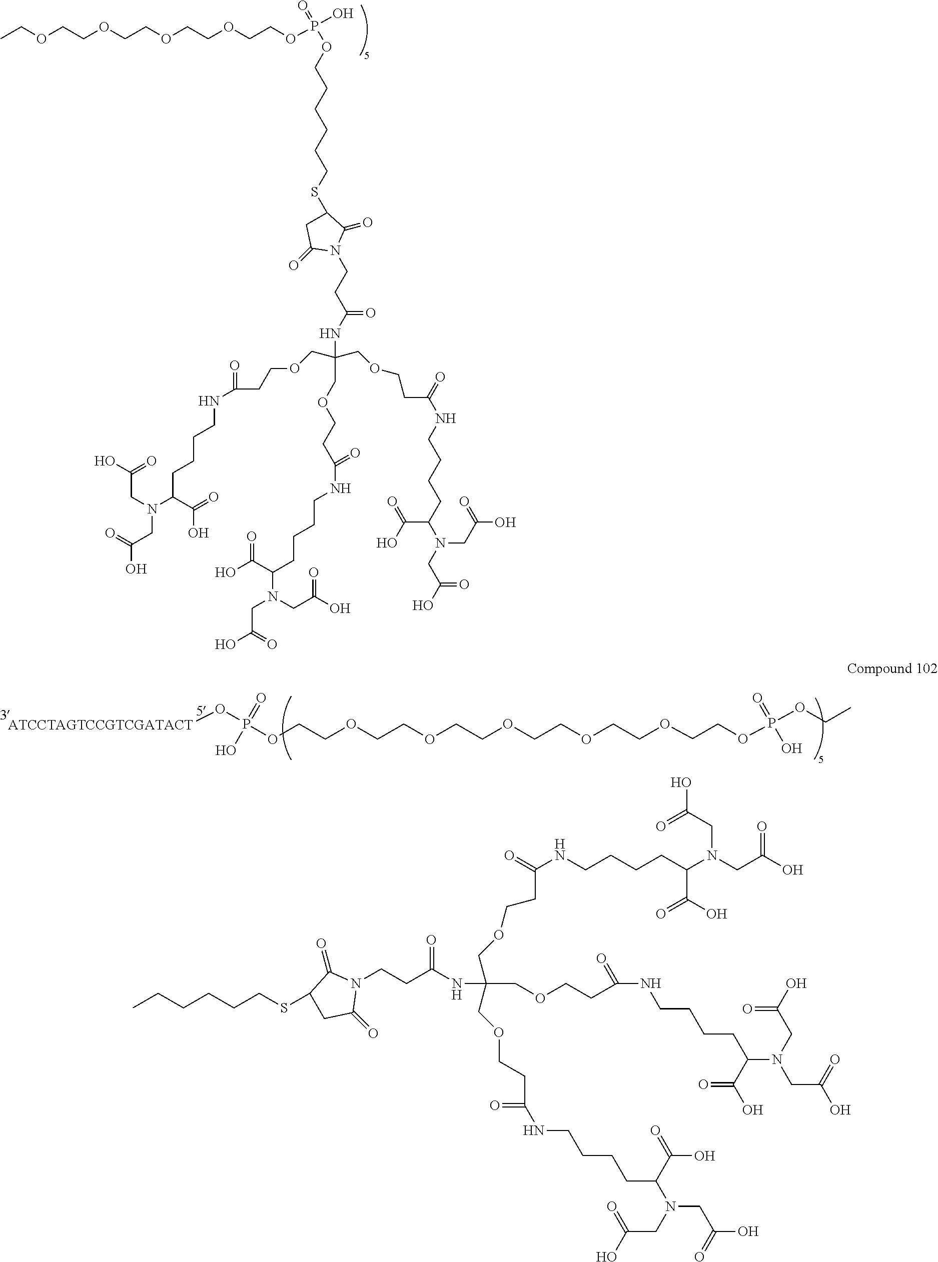

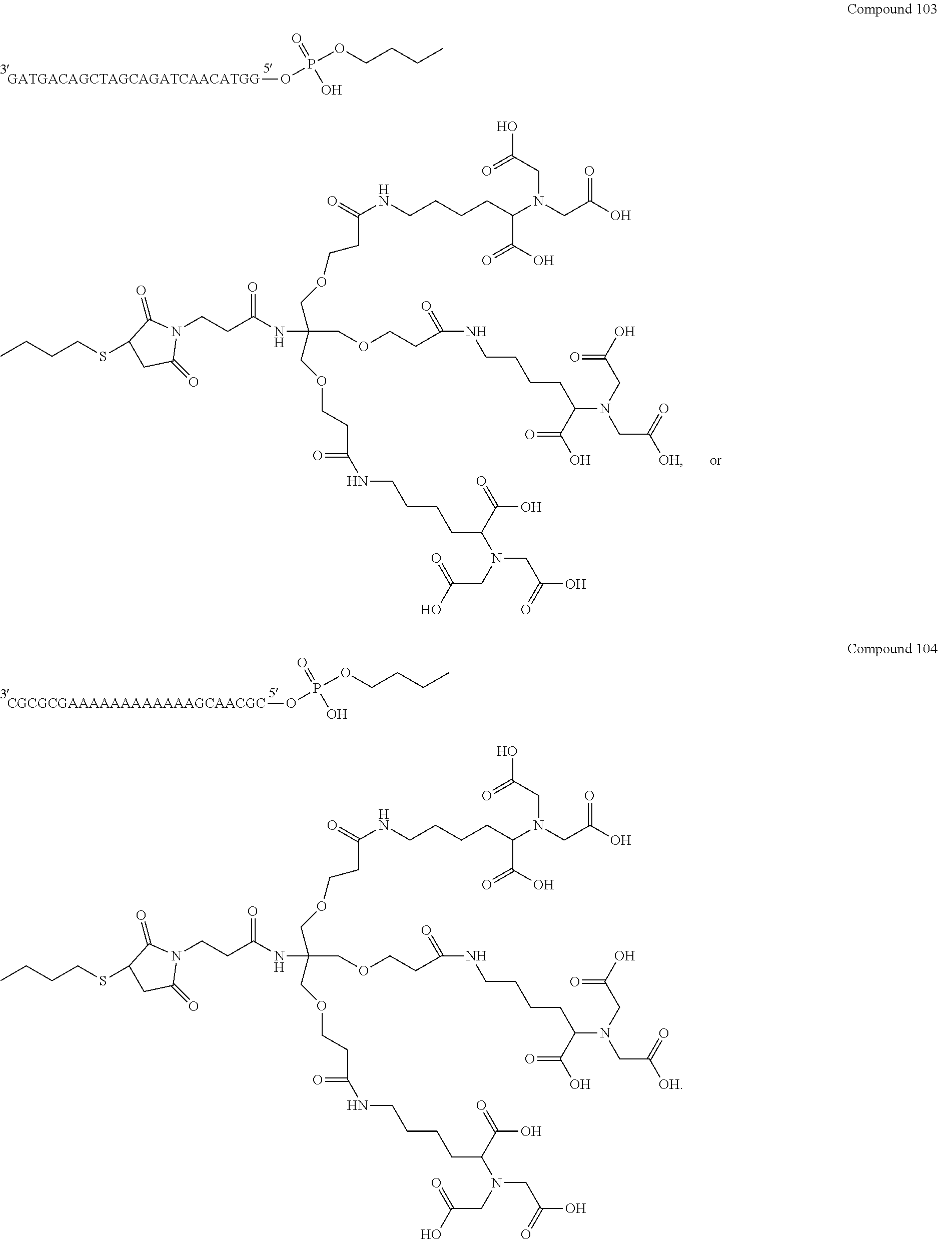

147. The system of claim 138,
wherein said second oligonucleotide is longer than said first oligonucleotide, said second oligonucleotide comprises a toehold region, or a combination thereof,
wherein said synthetic agent of said second compound is bound to the 3′ end or to the 5′ end of said second oligonucleotide,
wherein said synthetic agent of said second compound comprises a molecular marker, a labeling moiety, a fluorescent dye, an adhesion molecule, a cancer cell binder, a protein binder, a protein ligand, an anticancer agent, a surface binder, a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof,
wherein said second compound further comprises a second labeling moiety,
or any combination thereof.

148. The system of claim 147,
wherein said dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mica), dabcyl, NBD, Nile blue, TAMRA, BODIPY, FITC or derivative thereof;
said protein binder comprises a biotin or a folate;
said adhesion molecule comprises a folate;
said surface binder is an abiotic surface binder;
said surface binder comprises a thiol group (HS), a Si-halogen group, and/or a Si—O bond;
said cancer cell binder comprises a folate;
said second labeling moiety is bound to the 3′ end or to the 5′ end of said second oligonucleotide, directly or through a fourth linker,
and/or
wherein said second labeling moiety comprises a fluorescent dye.

149. The system of claim 148, wherein said fluorescent dye is selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof.

150. The system of claim 138, wherein the second compound is represented by the structure of the following compounds:

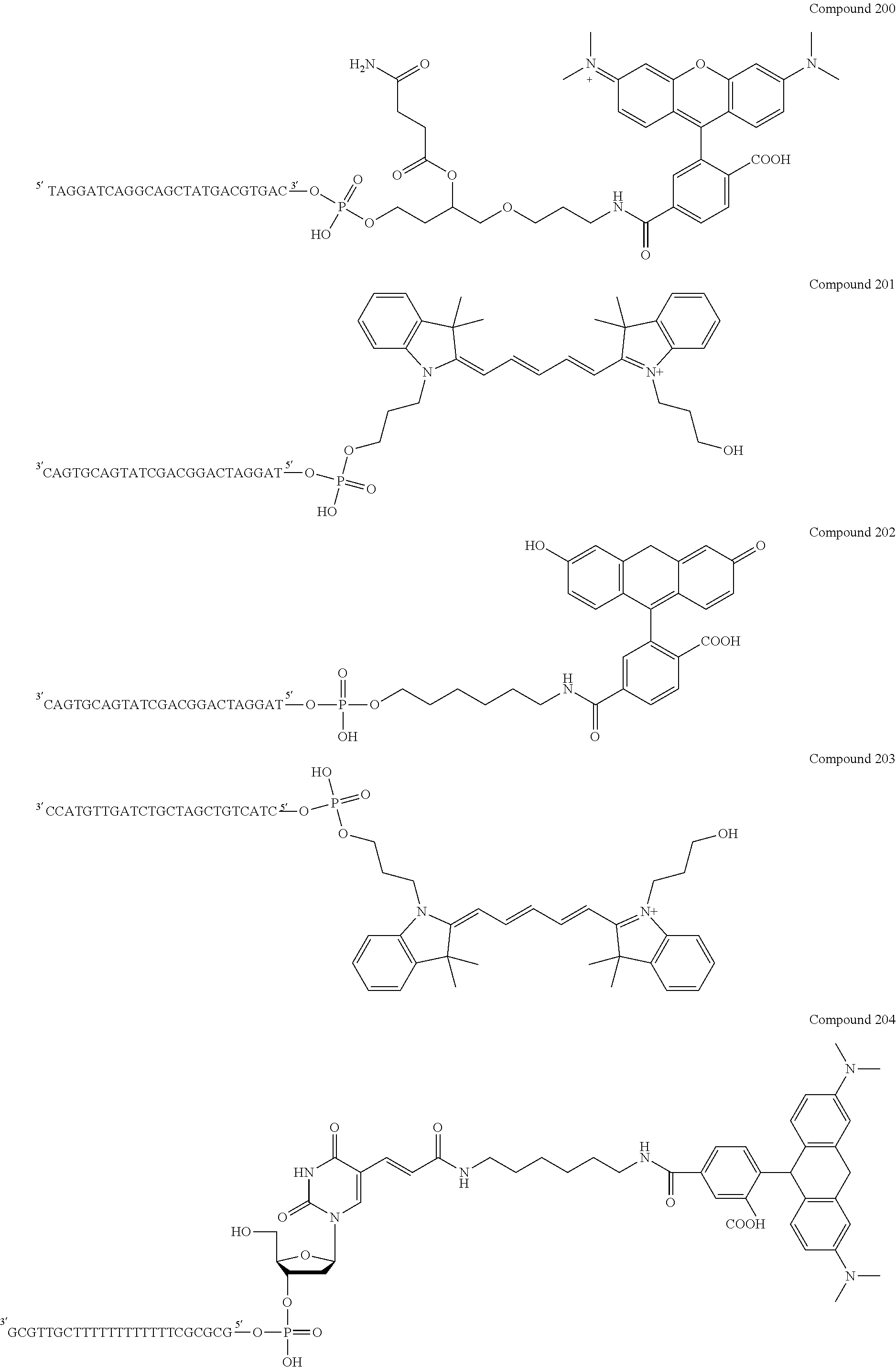

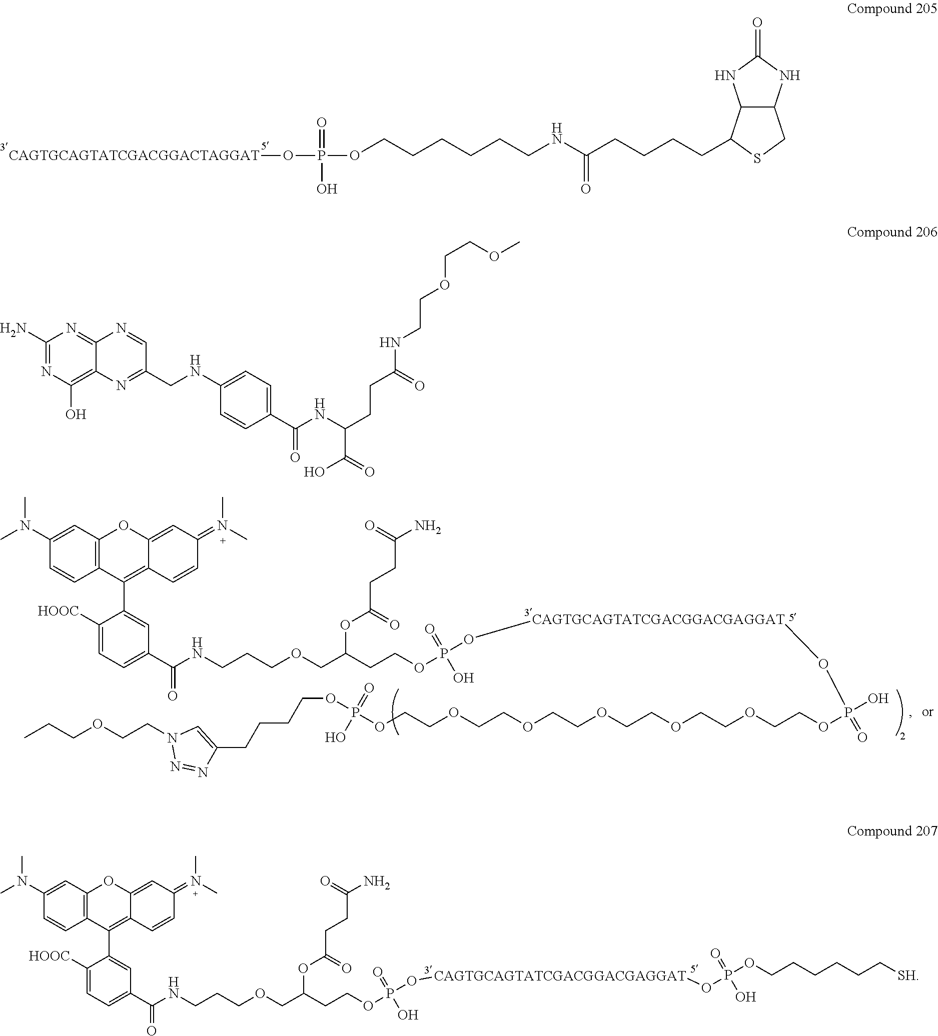

151. The system of claim 13838, further comprising a third compound comprising a third oligonucleotide (ODN-3),
wherein said third oligonucleotide is complementary to said second oligonucleotide, and/or
wherein said third oligonucleotide comprises higher affinity to said second oligonucleotide than the affinity of said second oligonucleotide to said first oligonucleotide.

152. A recombinant cell ectopically expressing a polypeptide, wherein said polypeptide comprises a membranal anchoring domain and an extracellular binding domain, said extracellular binding domain bound to
a. a first compound comprising a first oligonucleotide (ODN-1) covalently bound to a binder, either directly or through a first linker, said binder comprising affinity to said extracellular binding domain,
b. a second compound comprising a second oligonucleotide (ODN-2) covalently bound to a bioactive moiety, either directly or through a second linker, wherein said second oligonucleotide is complementary to said first oligonucleotide.

153. The cell of claim 152,
wherein said polypeptide is a cell surface protein (CSP),
wherein the cell is selected from a group comprising eukaryotes, prokaryotes, mammalian cells, plant cells, human cells, and bacteria,
wherein said membranal anchoring domain comprises a transmembranal protein or a part of it, an artificial polypeptide, or a combination thereof,
wherein said extracellular domain comprises an affinity tag,
wherein said binder comprises a His-tag specific binder,
or any combination thereof.

154. The cell of claim 153Error! Reference source not found., wherein
said transmembranal protein comprises an outer membrane protein C (OmpC); receptor tyrosine kinases (RTKs); Ion channel linked receptors; Enzyme-linked receptors; G protein-coupled receptors or any combination thereof,
said affinity tag comprises a poly-histidine peptide (6×-His-tag, 10×-His-tag, His-tag), a tetra cysteine peptide (CCPGCC, TC tag),
or a combination thereof.

155. The cell of claim 153Error! Reference source not found., wherein said His-tag specific binder comprises a moiety represented by the structure of formula E: 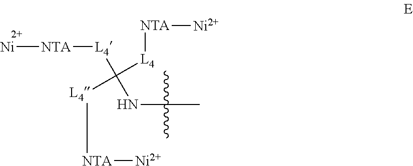
wherein
L₄, L₄′, and L₄″ are each independently a substituted or unsubstituted linear or branched alkyd chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof;
formula E(a): 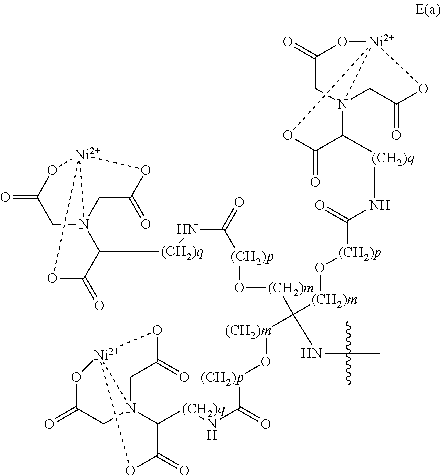
wherein
m, p and q are each independently an integer number between 1 and 8; or
formula E(b):

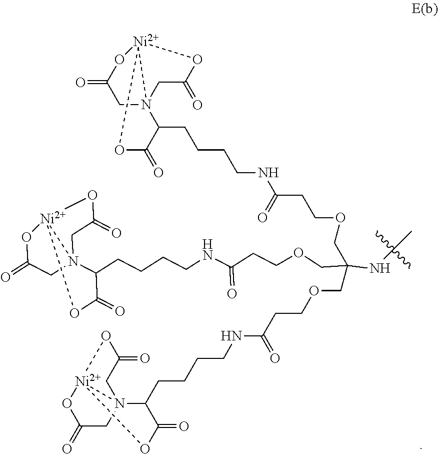

156. The cell of claim 152, wherein said first linker comprises at least one polyethyleneglycol (PEG) moiety, at least one phosphate moiety, at least one thioalkyl moiety or any combination thereof;
or said first linker is represented by the following formula:
—[(CH₂O)ₖ—PO₃H]ₗ—(CH₂)_{w}—S—
wherein
k and l are each independently an integer number between 0 and 10; and
w is an integer number between 1 and 10.

157. The cell of claim 152, wherein said first compound further comprises a labeling moiety.

158. The cell of claim 157, wherein said labeling moiety is bound to the 3′ end or to the 5′ end of said first oligonucleotide, directly or through a third linker, and/or wherein said labeling moiety comprises a fluorescent dye.

159. The cell of claim 158, wherein said fluorescent dye is selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof.

160. The cell of claim 152, wherein said first compound is represented by the structure of the nickel complexes of the following compounds:

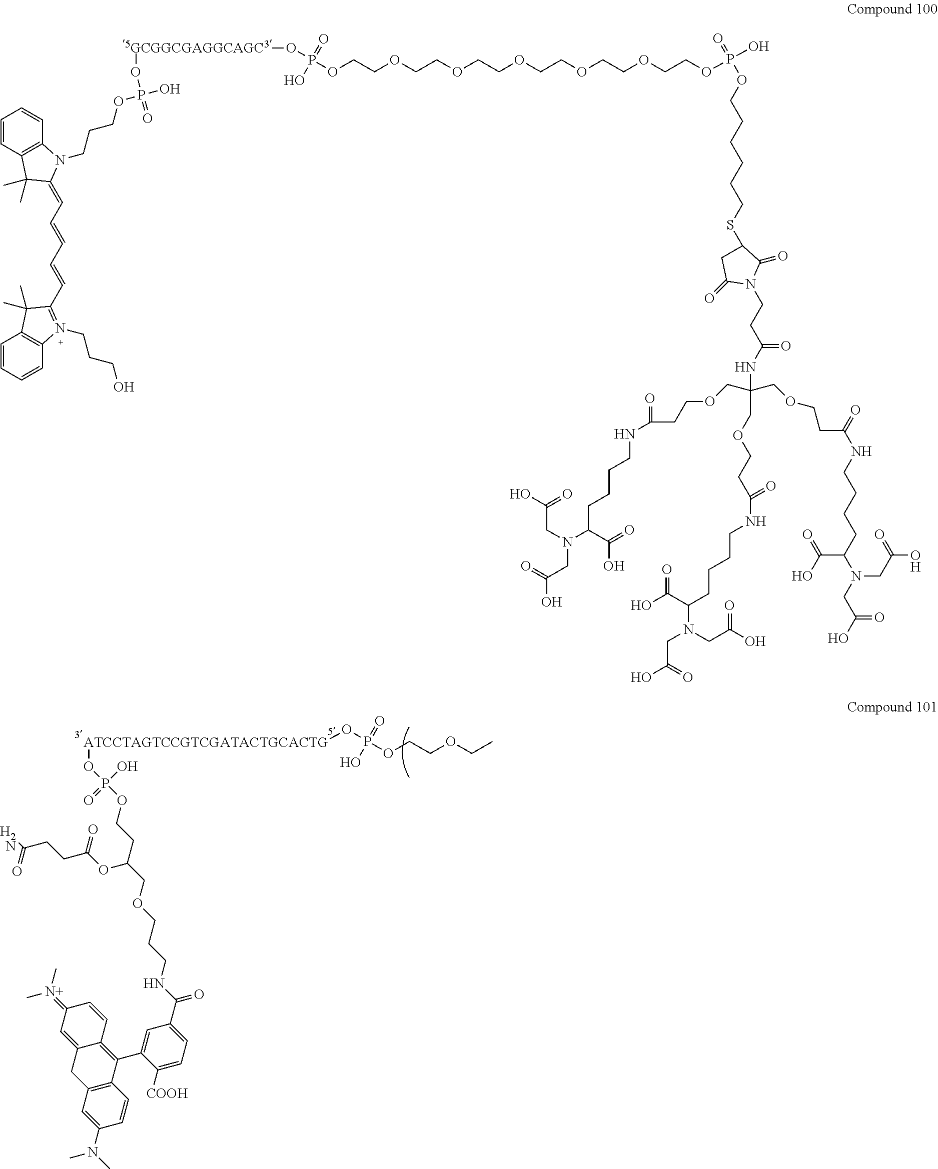

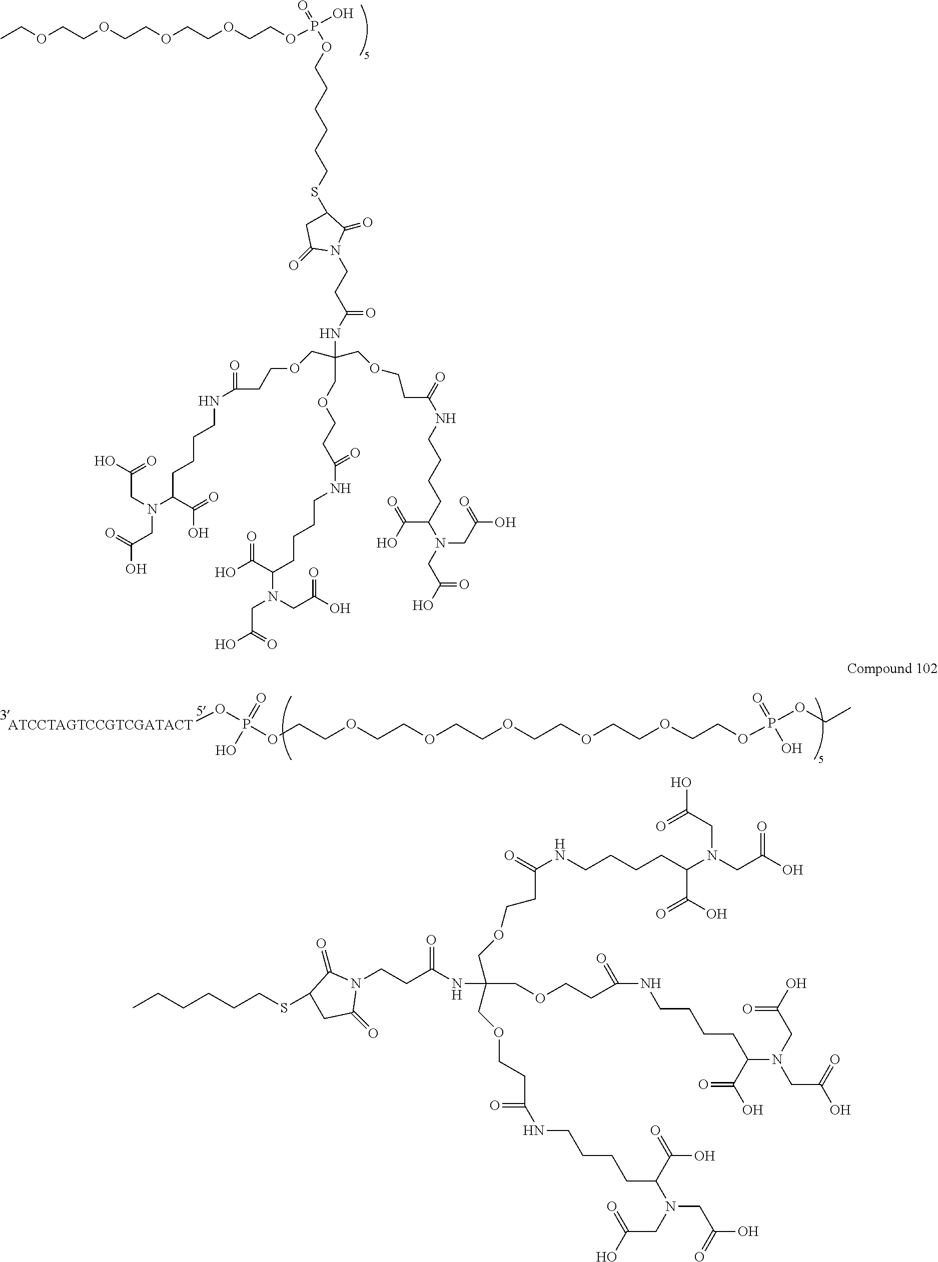

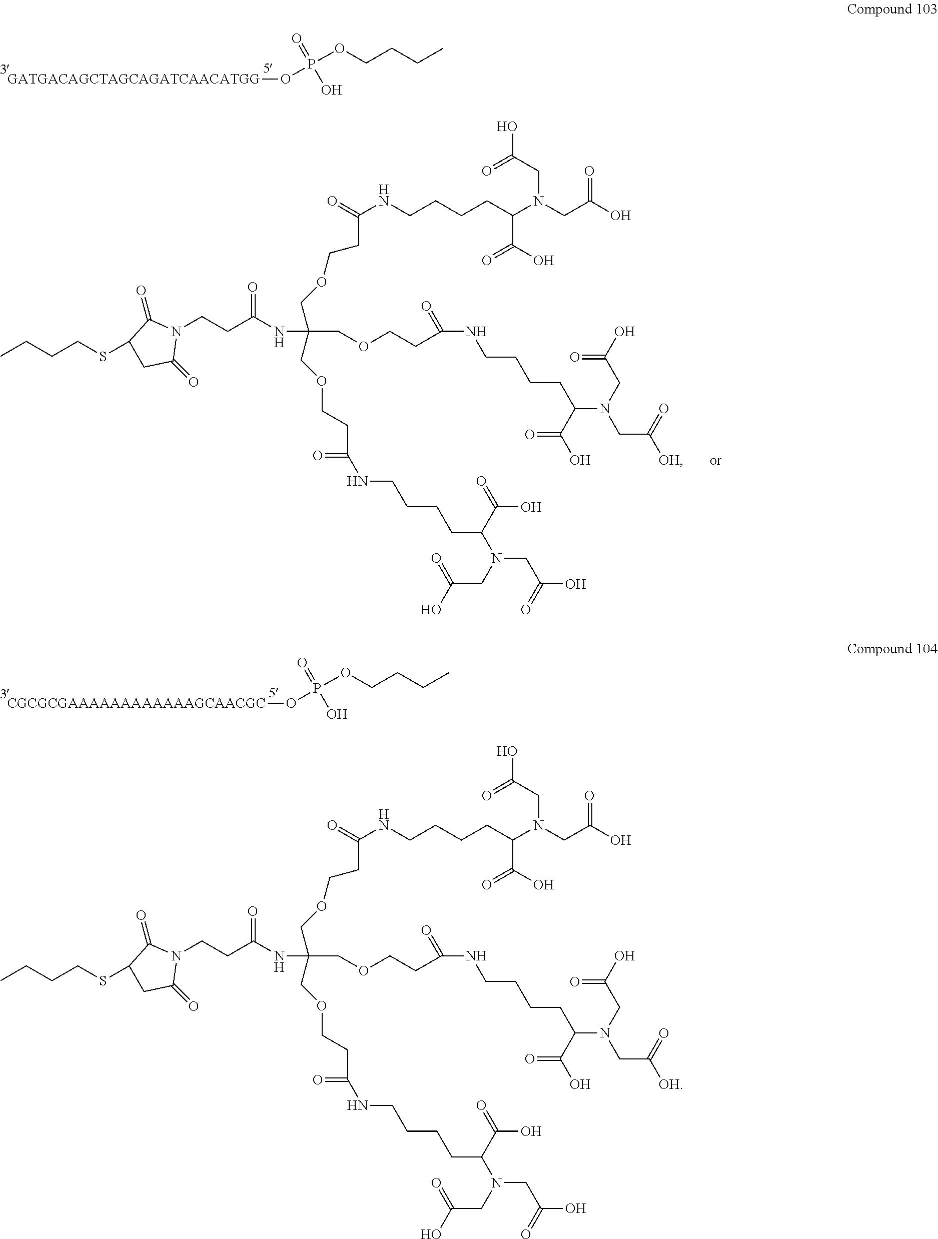

161. The cell of claim 152,
wherein said second oligonucleotide is longer than said first oligonucleotide, comprises a toehold region, or combination thereof,
wherein said synthetic agent of said second compound is bound to the 3′ end or to the 5′ end of said second oligonucleotide,
wherein said synthetic agent of said second compound comprises a molecular marker, a labeling moiety, a fluorescent dye, an adhesion molecule, a cancer cell binder, a protein binder, a protein ligand, an anticancer agent, a surface binder, a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof,
wherein said second compound further comprises a second labeling moiety,
or any combination thereof.

162. The cell of claim 161,
wherein
said dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof;
said protein binder comprises a biotin or a folate;
said adhesion molecule comprises a folate;
said surface binder is an abiotic surface binder;
said surface binder comprises a thiol group (HS), a Si-halogen group, a and/or a Si—O bond;
said cancer cell binder comprises a folate;
said second labeling moiety is bound to the 3′ end or to the 5′ end of said second oligonucleotide, directly or through a fourth linker,
and/or said second labeling moiety comprises a fluorescent dye.

163. The cell of claim 162, wherein said fluorescent dye is selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof.

164. The cell of claim 152, wherein the second compound is represented by the structure of the following compounds:

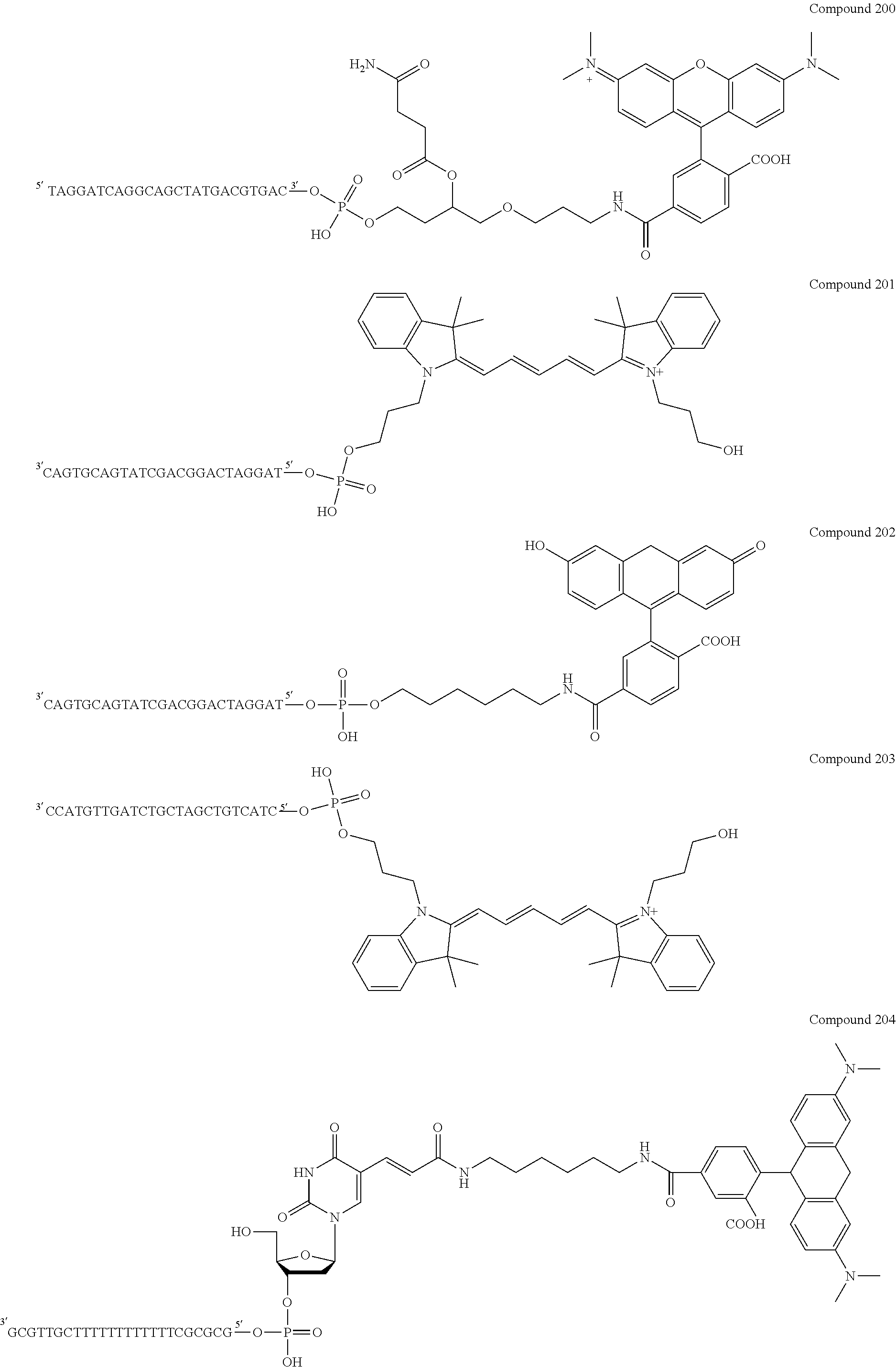

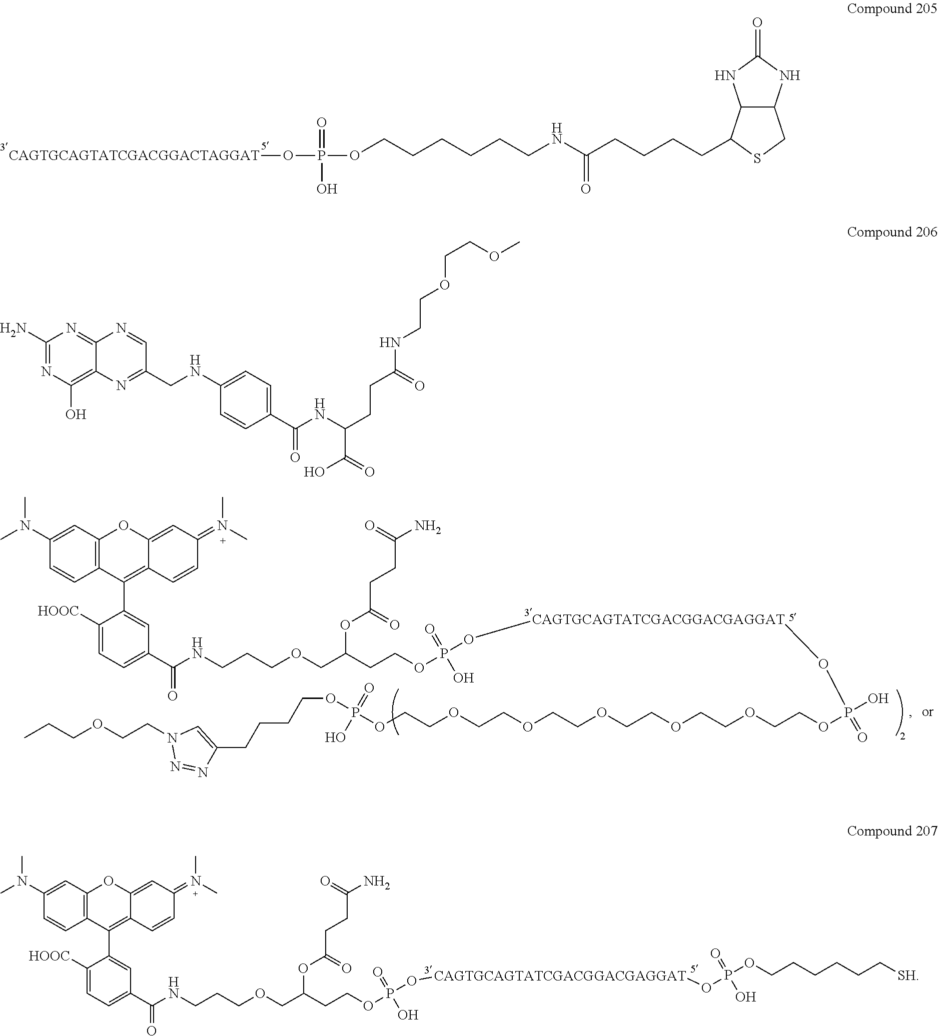

165. An artificial receptor, capable of binding a His-tagged protein, comprising
a. a first compound comprising a first oligonucleotide (ODN-1) covalently bound to a His-tag binder, either directly or through a first linker, said binder comprises a moiety represented by the structure of formula E: 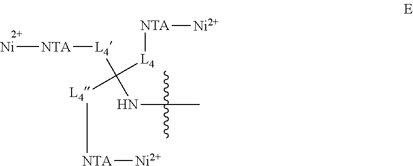
wherein
L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof; and
b. a second compound comprising a second oligonucleotide (ODN-2) covalently bound to a synthetic agent, either directly or through a second linker, said second oligonucleotide is complementary to said first oligonucleotide.

166. The artificial receptor of claim 165,
wherein said receptor does not perturb the function of a living cell;
and/or said receptor can be reversibly modified.

167. The artificial receptor of claim 165,
wherein L₄, L₄′, and L₄″ are each independently-(CH₂)_{q}—NHCO—(CH₂)ₚ—O—(CH₂)ₘ—,
wherein m, p and q are each independently an integer between 1 and 6;
wherein said formula (E) is represented by the structure of formula E(a): 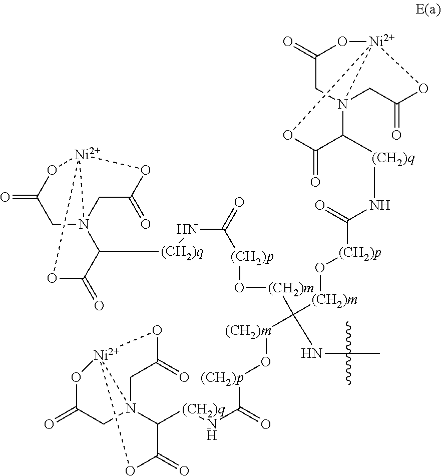
wherein
m, p and q are each independently an integer number between 1 and 8; or
by the structure of formula E(b):

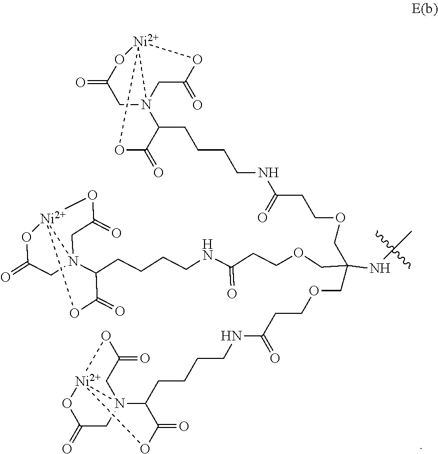

168. The artificial receptor of claim 165,
wherein said first linker comprises at least one polyethyleneglycol (PEG) moiety, at least one phosphate moiety, at least one thioalkyl moiety or any combination thereof;
or wherein said first linker is represented by the following formula:
—[(CH₂O)ₖ—PO₃H]ₗ—(CH₂)_{w}—S—
wherein
k and l are each independently an integer number between 0 and 10; and
w is an integer number between 1 and 10.

169. The artificial receptor of claim 165, wherein said first compound further comprises a labeling moiety.

170. The artificial receptor of claim 169, wherein said labeling moiety is bound to the 3′ end or to the 5′ end of said first oligonucleotide, directly or through a third linker, and/or said labeling moiety comprises a fluorescent dye.

171. The artificial receptor of claim 170, wherein said fluorescent dye is selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof.

172. The artificial receptor of claim 165, wherein said first compound is represented by the structure of the nickel complexes of the following compounds:

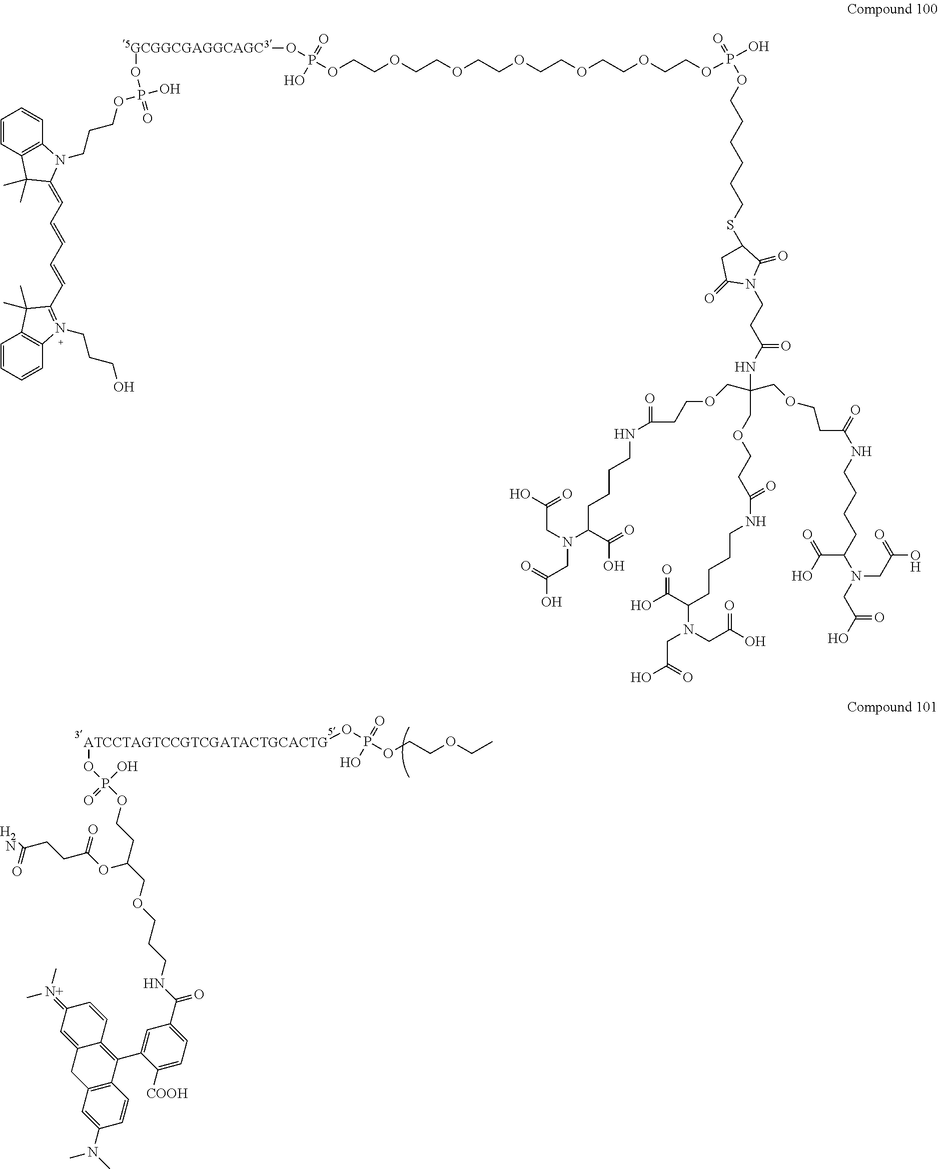

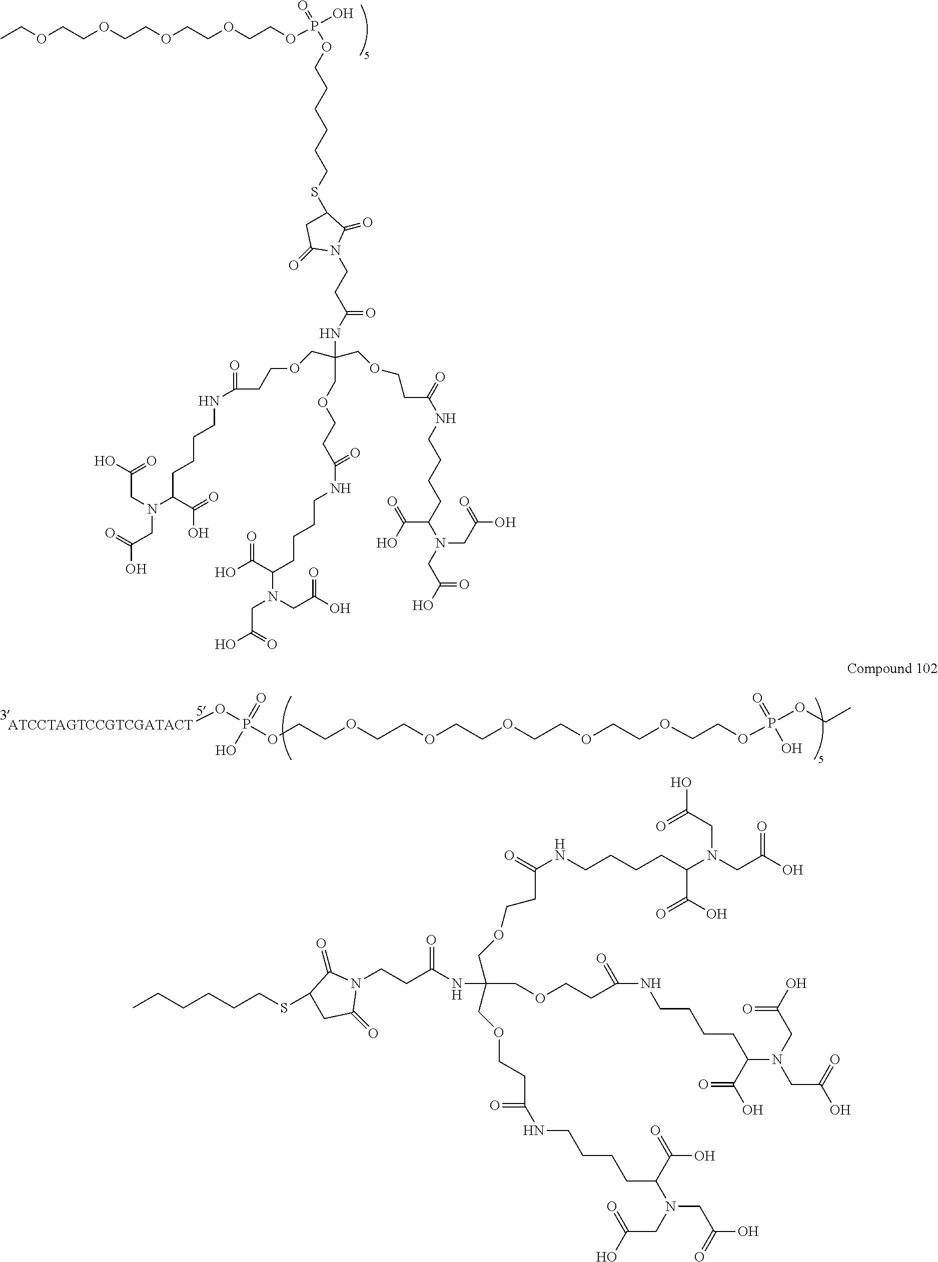

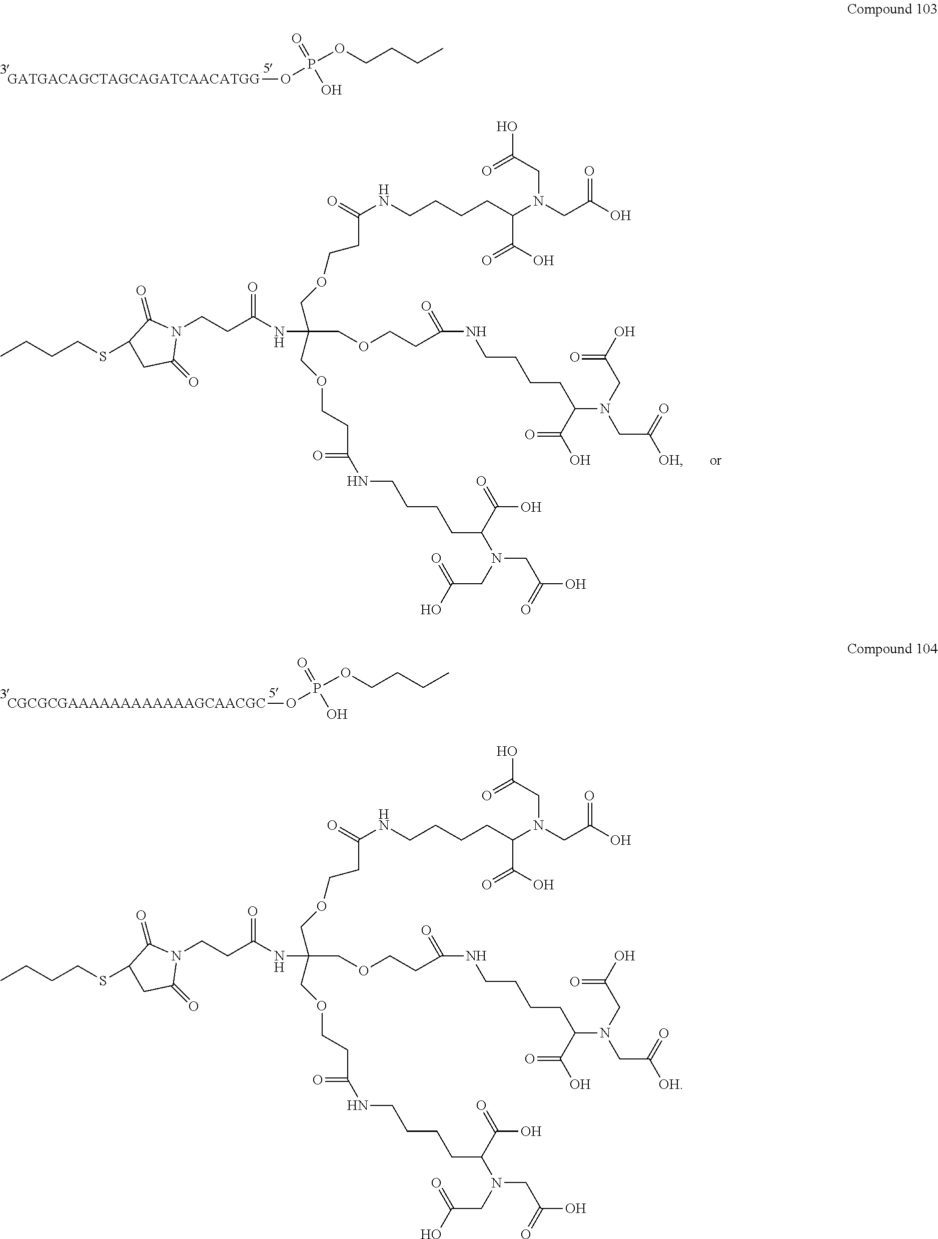

173. The artificial receptor of claim 165,
wherein said second oligonucleotide is longer than said first oligonucleotide, comprises a toehold region, or combination thereof,
wherein said synthetic agent of said second compound is bound to the 3′ end or to the 5′ end of said second oligonucleotide,
wherein said synthetic agent of said second compound comprises a molecular marker, a labeling moiety, a fluorescent dye, an adhesion molecule, a cancer cell binder, a protein binder, a protein ligand, an anticancer agent, a surface binder, a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof,
wherein said second compound further comprises a second labeling moiety,
or any combination thereof.

174. The artificial receptor of claim 173,
wherein
said dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMPA, BODIPY, FITC or derivative thereof;
said protein binder comprises a biotin or a folate;
said adhesion molecule comprises a folate;
said surface binder is an abiotic surface binder;
said surface binder comprises a thiol group (US), a Si-halogen group, or a Si—O bond;
said cancer cell binder comprises a folate,
said second labeling moiety is bound to the 3′ end or to the 5′ end of said second oligonucleotide, directly or through a fourth linker,
and/or said second labeling moiety comprises a fluorescent dye.

175. The artificial receptor of claim 174, wherein said fluorescent dye is selected from a group comprising dansyl, fluorescein (6-FAM), YAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof.

176. The artificial receptor of claim 165, wherein the second compound is represented by the structure of the following compounds:

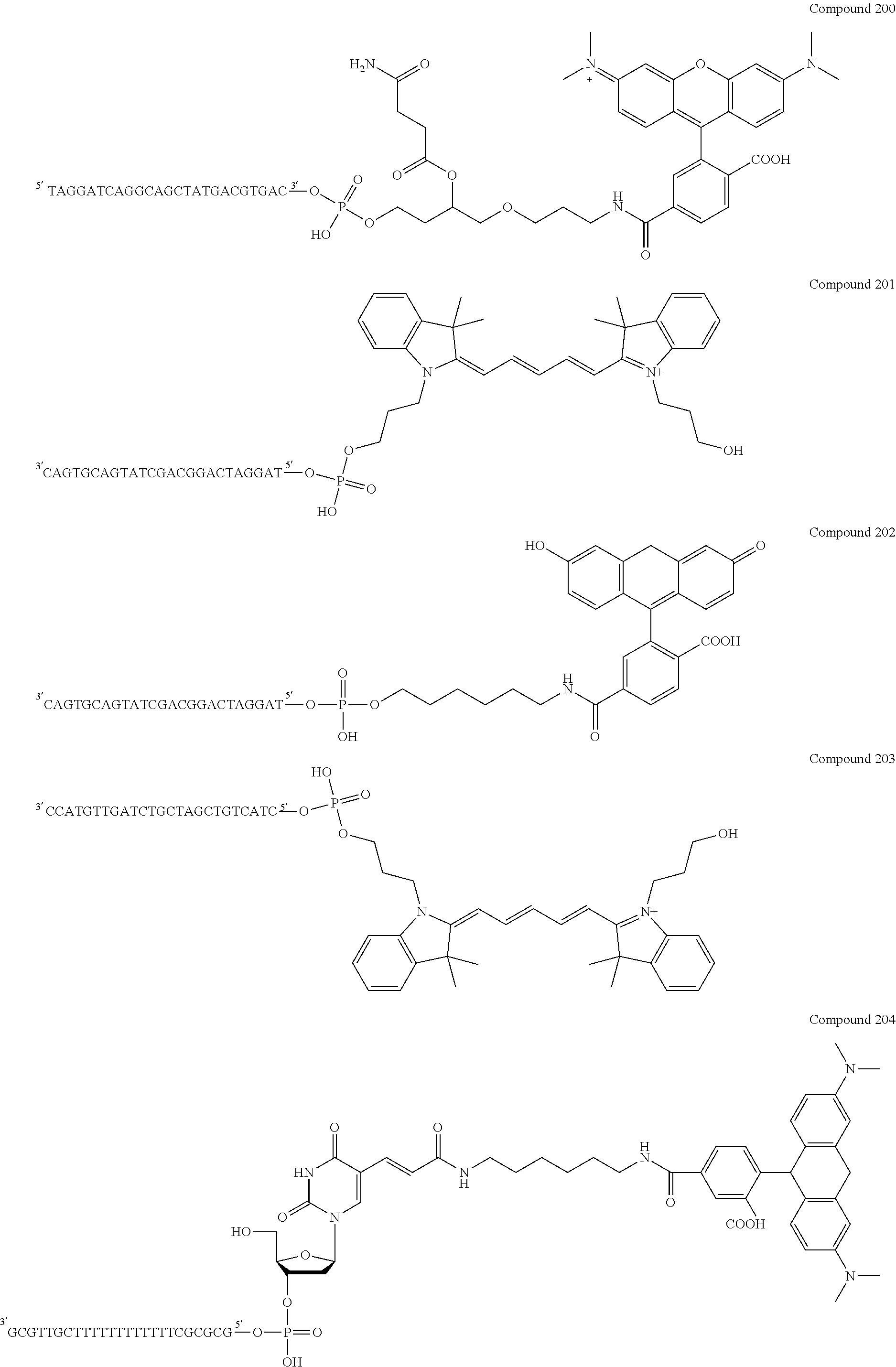

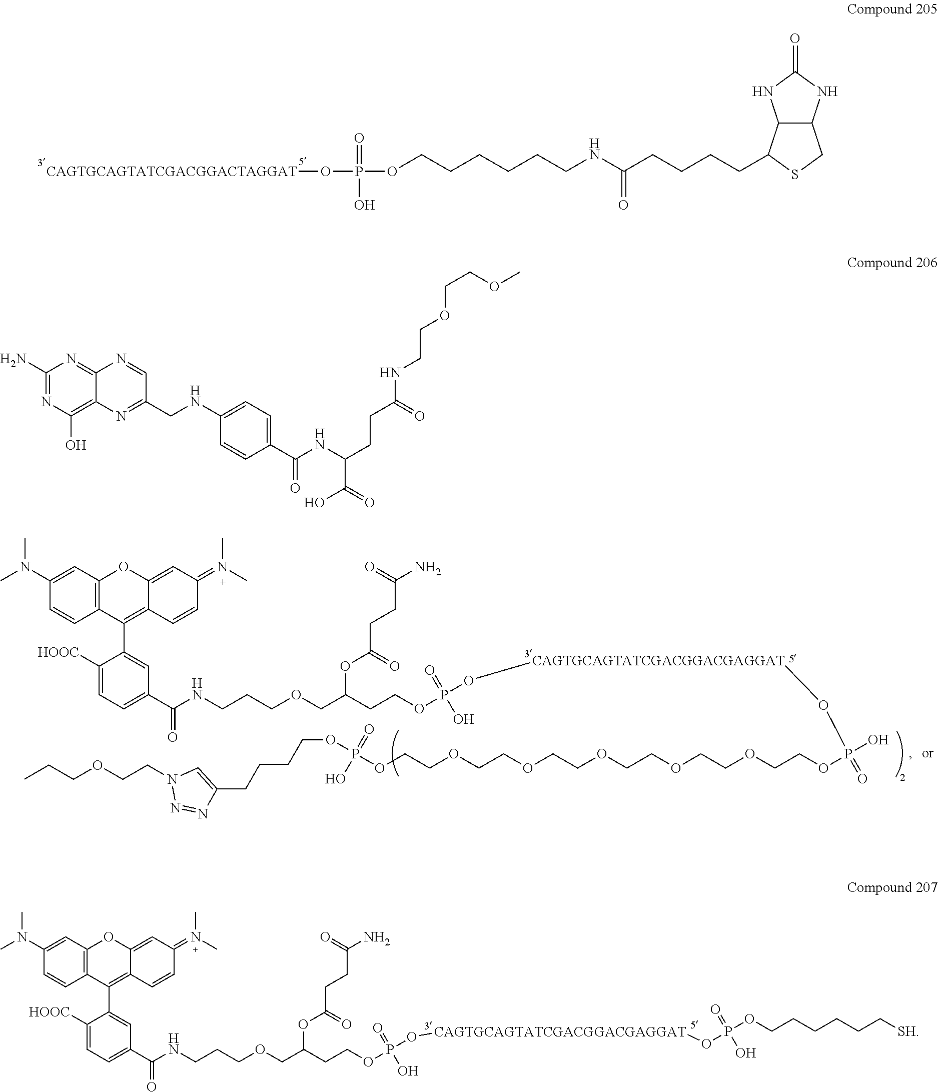

177. The artificial receptor of claim 165, further comprising a third compound comprising a third oligonucleotide (ODN-3),
wherein said third oligonucleotide is complementary to said second oligonucleotide,
and/or wherein said third oligonucleotide comprises higher affinity to said second oligonucleotide than the affinity of said second oligonucleotide to said first oligonucleotide.

178. A method for decorating a cell with a synthetic agent, said method comprises:
a. ectopically expressing in said cell a polypeptide, wherein said polypeptide comprises a membranal anchoring domain and an extracellular binding domain,
b. incubating the cell of (a) with a first compound comprising a first oligonucleotide (ODN-1) covalently bound to a binder, either directly or through a first linker, said binder comprising affinity to said extracellular binding domain, and
c. incubating the cell of (b) with a second compound comprising a second oligonucleotide (ODN-2) covalently bound to a synthetic agent, either directly or through a second linker, wherein said second oligonucleotide is complementary to said first oligonucleotide;
thereby decorating said cell with said synthetic agent;
a method for binding a first cell to a second cell, said method comprises:
a. ectopically expressing in the first cell a polypeptide, wherein said polypeptide comprises a membranal anchoring domain and an extracellular binding domain,
b. incubating the cell of (a) with a first compound comprising a first oligonucleotide (ODN-1) covalently bound to a binder, either directly or through a first linker, said binder comprises affinity to said extracellular binding domain, and
c. incubating the cell of (b) with a second compound comprising a second oligonucleotide (ODN-2) covalently bound to an adhesion molecule, either directly or through a second linker, wherein said second oligonucleotide is complementary to said first oligonucleotide, and said adhesion molecule comprises affinity to a compound present on the surface of said second cell,
d. incubating said first cell with said second cell,
thereby binding said first cell to said second cell;
a method for adhering a cell to an abiotic surface, said method comprises:
a. ectopically expressing in a cell a polypeptide, wherein said polypeptide comprises a membranal anchoring domain and an extracellular binding domain,
b. incubating the cell of (a) with a first compound comprising a first oligonucleotide (ODN-1) covalently bound to a binder, either directly or through a first linker, said binder comprises affinity to said extracellular binding domain, and
c. incubating the cell of (b) with a second compound comprising a second oligonucleotide (ODN-2) covalently bound to an abiotic surface binder, either directly or through a second linker, wherein said second oligonucleotide is complementary to said first oligonucleotide, and said surface binder is capable of binding to said surface, and
d. applying said cell to said surface under conditions sufficient for the binding of said abiotic surface binder to said abiotic surface,
thereby adhering said cell to said abiotic surface;
a method for inducing luminescence in a cell, said method comprises:
a. ectopically expressing in a cell a first polypeptide, wherein said polypeptide comprises a membranal anchoring domain and an extracellular binding domain,
b. incubating the cell of (a) with a first compound, comprising a first oligonucleotide (ODN-1) covalently bound to a binder, either directly or through a first linker, said binder comprises affinity to said extracellular binding domain, and
c. incubating the cell of (b) with a second compound, comprising a second oligonucleotide (ODN-2) covalently bound to a luminescent molecule, either directly or through a second linker, wherein the second oligonucleotide is complementary to the first oligonucleotide,
thereby inducing luminescence in said cell: or
a method for binding a cell to a protein of interest (POI), said method comprises:
a. ectopically expressing in a cell a polypeptide, wherein said polypeptide comprises a membranal anchoring domain and an extracellular binding domain,
b. incubating the cell of (a) with a first compound according to this invention, comprising a first oligonucleotide (ODN-1) covalently bound to a binder, either directly or through a first linker, said binder comprises affinity to said extracellular binding domain, and
c. incubating the cell of (b) with a second compound according to this invention, comprising a second oligonucleotide (ODN-2) covalently bound to a protein binder, either directly or through a second linker, wherein said second oligonucleotide is complementary to said first oligonucleotide, and said protein binder is selective to said POI, and
d. incubating said cell with said POI,
thereby binding said cell to said POI.

179. The method of claim 178, wherein
said method for decorating a cell with a synthetic agent, is for decorating the cell's surface, the cell's membrane, or combination thereof;
said second cell of said method for binding a first cell to a second cell, expresses an extracellular protein receptor on its surface,
said second cell of said method for binding a first cell to a second cell, is a cancer cell,
said adhesion molecule of said method for binding a first cell to a second cell, is a protein binder,
said abiotic surface binder of said method for adhering a cell to an abiotic surface, is a C₁-C₂₀ thioalkyl,
said surface of said method for adhering a cell to an abiotic surface, is a material selected from gold, glass, a doped glass, indium tin oxide (ITO)-coated glass, silicon, a doped silicon, Si(100), Si(111), SiO₂, SiH, silicon carbide mirror, quartz, a metal, metal oxide, a mixture of metal and metal oxide, group IV elements, mica, a polymer such as polyacrylamide and polystyrene, a plastic, a zeolite, a clay, wood, a membrane, an optical fiber, a ceramic, a metalized ceramic, an alumina, an electrically-conductive material, a semiconductor, steel or a stainless steel;
said luminescent molecule of said method for inducing luminescence in a cell, is a fluorescent dye,
said protein binder of said method for binding a cell to a protein of interest (POI), is a small molecule ligand, a peptide, a polypeptide, a protein, or a part thereof;
said polypeptide of said methods is a cell surface protein (CSP),
said recombinant cell is selected from a group comprising eukaryotes, prokaryotes, mammalian cells, plant cells, human cells, and bacteria,
said membranal anchoring domain of said methods comprises a transmembranal protein or a part of it, an artificial polypeptide, or a combination thereof,
said extracellular domain of said methods comprises an affinity tag,
said binder of said methods comprises a His-tag specific binder,
said first linker of said methods comprises at least one polyethyleneglycol (PEG) moiety, at least one phosphate moiety, at least one thioalkyl moiety or any combination thereof,
said first compound of said methods further comprises a labeling moiety,
or any combination thereof.

180. The method of claim 179, wherein said His-tag specific binder comprises a moiety represented by the structure of formula E: 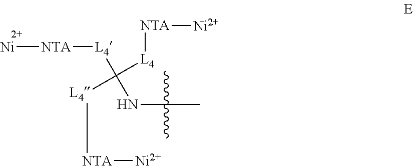
wherein
L₄, L₄′, and L₄″ are each independently a substituted or unsubstituted linear or branched alkyd chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof,
formula E(a): 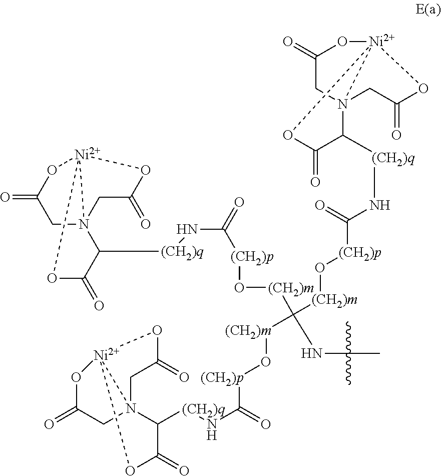
wherein
m, p and q are each independently an integer number between 1 and 8, or formula E(b):

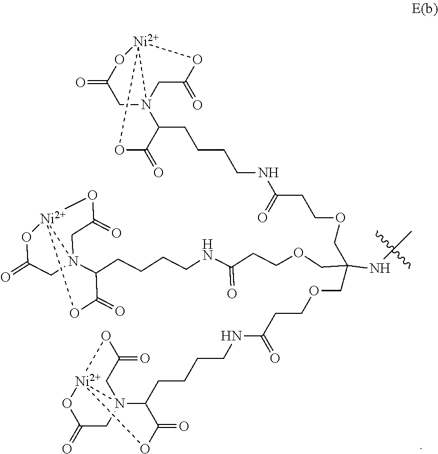

181. The method of claim 178, wherein said first linker is represented by the following formula:
—[(CH₂)ₖ—PO₃H]ₗ—(CH₂)_{w}—S—
wherein
k and l are each independently an integer number between 0 and 10; and
w is an integer number between 1 and 10.

182. The method of claim 178, wherein said first compound further comprises a labeling moiety.

183. The method of claim 182, wherein said labeling moiety is bound to the 3′ end or to the 5′ end of said first oligonucleotide, directly or through a third linker, and/or wherein said labeling moiety comprises a fluorescent dye.

184. The method of claim 183, wherein said fluorescent dye is selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof.

185. The method of claim 178, wherein said first compound is represented by the structure of the nickel complexes of the following compounds:

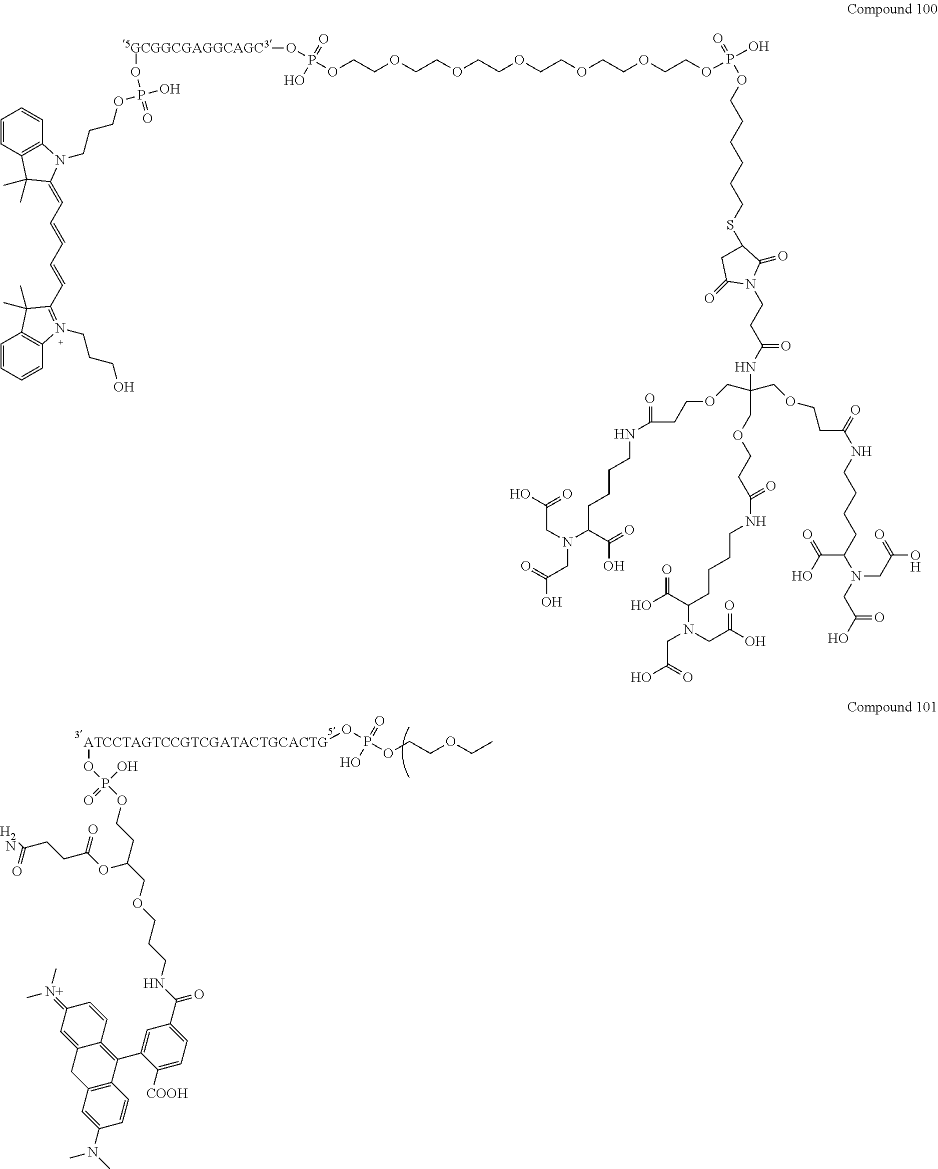

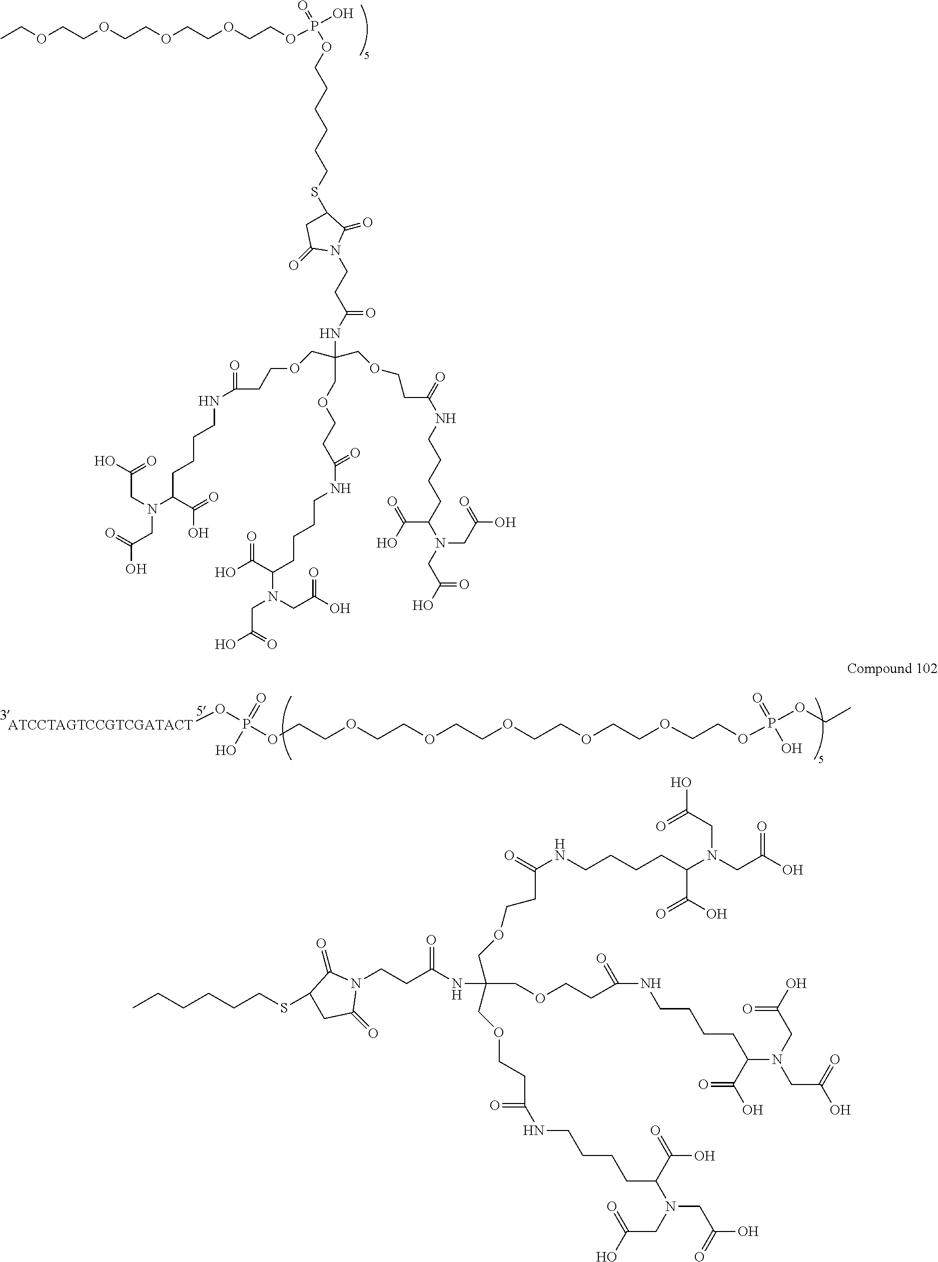

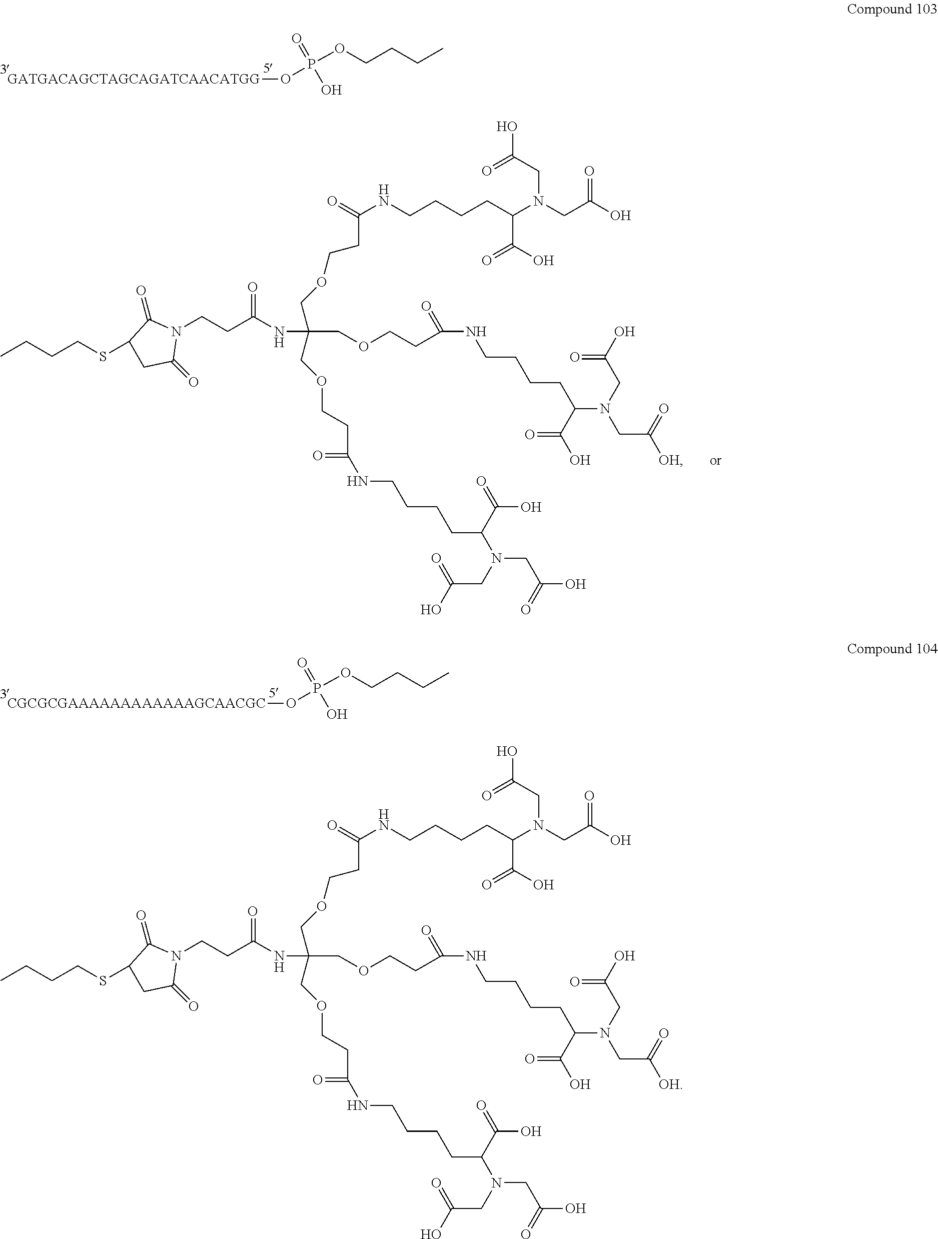

186. The method of claim 178,
wherein said second oligonucleotide is longer than said first oligonucleotide, comprises a toehold region, or combination thereof;
wherein said synthetic agent of said second compound comprises a molecular marker, a labeling moiety, a fluorescent dye, an adhesion molecule, a cancer cell binder, a protein binder, a protein ligand, an anticancer agent, a surface binder, a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof,
wherein said second compound further comprises a second labeling moiety, or any combination thereof.

187. The method of claim 186, wherein
said dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof;
said protein binder comprises a biotin or a folate;
said adhesion molecule comprises a folate;
said surface binder is an abiotic surface binder;
said surface binder comprises a thiol group (HS), a Si-halogen group, or a Si—O bond; or
said cancer cell binder comprises a folate;
said second labeling moiety is bound to the 3′ end or to the 5′ end of said second oligonucleotide, directly or through a fourth linker, and/or
said second labeling moiety comprises a fluorescent dye.

188. The method of claim 187, wherein said fluorescent dye is selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC or derivative thereof.

189. The method of claim 178, wherein the second compound is represented by the structure of the following compounds:

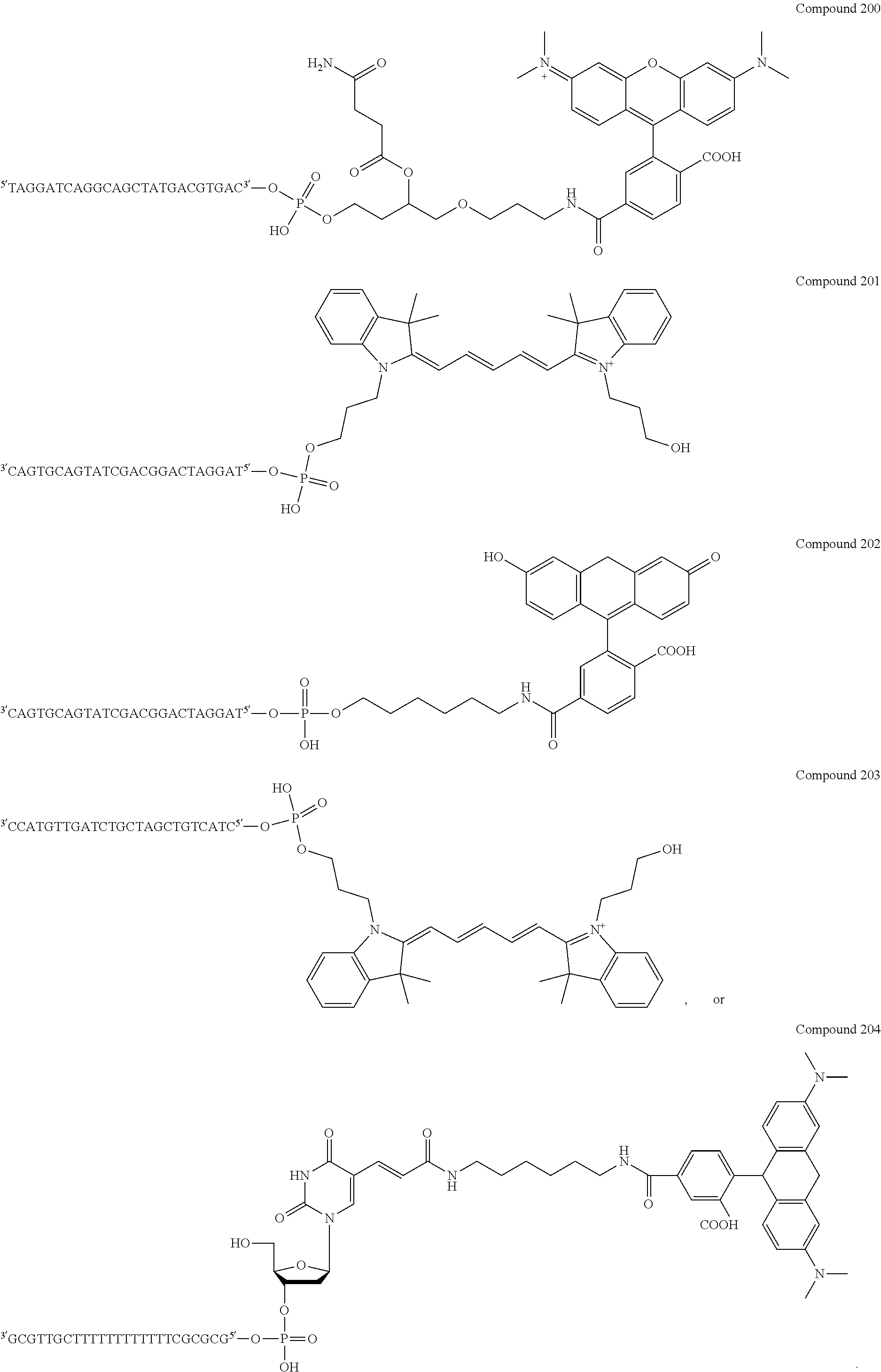

190. A kit comprising:
a. a recombinant cell ectopically expressing a polypeptide according to this invention, wherein said polypeptide comprises a membranal anchoring domain and an extracellular binding domain, said extracellular binding domain bound to
b. a first compound according to this invention, comprising a first oligonucleotide (ODN-1) covalently bound to a binder according to this invention, either directly or through a first linker, said binder comprises affinity to said extracellular binding domain,
c. a second compound according to this invention, comprising a second oligonucleotide (ODN-2) covalently bound to a synthetic agent, either directly or through a second linker, wherein said second oligonucleotide is complementary to said first oligonucleotide.


SEQUENCE LISTING (21 sequences)

SEQ ID NO: 1 - DNA, 13 bp, Artificial Sequence
  CY5-ODN-1
gcggcgaggcagc

SEQ ID NO: 2 - DNA, 25 bp, Artificial Sequence
  TAMRA-ODN-1
gtcacgtcatagctgcctgatccta

SEQ ID NO: 3 - DNA, 19 bp, Artificial Sequence
  ODN-1 and ODN-1a
tcatagctgcctgatccta

SEQ ID NO: 4 - DNA, 25 bp, Artificial Sequence
  ODN-1b
ggtacaactagacgatcgacagtag

SEQ ID NO: 5 - DNA, 25 bp, Artificial Sequence
  ODN-1c
cgcaacgaaaaaaaaaaaagcgcgc

SEQ ID NO: 6 - DNA, 25 bp, Artificial Sequence
  TAMRA-ODN-2
taggatcaggcagctatgacgtgac

SEQ ID NO: 7 - DNA, 25 bp, Artificial Sequence
  ODN-2 or ODN-2a
taggatcaggcagctatgacgtgac

SEQ ID NO: 8 - DNA, 24 bp, Artificial Sequence
  ODN-2b
tactgtcgatcgtctagttgtacc

SEQ ID NO: 9 - DNA, 25 bp, Artificial Sequence
  ODN-2c
gcgcgcttttttttttttcgttgcg

SEQ ID NO: 10 - DNA, 25 bp, Artificial Sequence
  ODN-3
gtcacgtcatagctgcctgatccta

SEQ ID NO: 11 - DNA, 54 bp, Artificial Sequence
  OmpC_FpET21
tttgtttaactttaagaaggagatatacatatgaaagttaaagtactgtccctc

SEQ ID NO: 12 - DNA, 52 bp, Artificial Sequence
  OmpC_RpET21
ttcctttcgggctttgttagcagccggatcttagaactggtaaaccagaccc

SEQ ID NO: 13 - protein, 11 aa, Artificial Sequence
  OmpC-(6His)1
SAGHHHHHHGT

SEQ ID NO: 14 - DNA, 48 bp, Artificial Sequence
  OmpC_His1
catcatcaccatggtacctctaaaggtaaaaacctgggtcgtggctac

SEQ ID NO: 15 - DNA, 60 bp, Artificial Sequence
  OmpC_His1R
atggtgatgatgatgatgacccgcggaggtaccatggtgatgatggtgatgacccgcgga

SEQ ID NO: 16 - protein, 22 aa, Artificial Sequence
  OmpC-(6His)2
SAGHHHHHHGTSAGHHHHHHGT

SEQ ID NO: 17 - DNA, 40 bp, Artificial Sequence
  OmpC_His2FInverse
caccatcacggtacctctaaaggtaaaaacctgggtcgtg

SEQ ID NO: 18 - DNA, 42 bp, Artificial Sequence
  OmpC_His2RInverse
gtgatggtgacccgcggaggtaccatggtgatgatggtgatg

SEQ ID NO: 19 - DNA, 40 bp, Artificial Sequence
  OmpC_His3FInverse
catcatcatggtacctctaaaggtaaaaacctgggtcgtg

SEQ ID NO: 20 - DNA, 42 bp, Artificial Sequence
  OmpC_His3RInverse
atgatgatgacccgcggaggtaccgtgatggtggtgatggtg

SEQ ID NO: 21 - protein, 33 aa, Artificial Sequence
  OmpC-(6His)3
SAGHHHHHHGTSAGHHHHHHGTSAGHHHHHHGT